(19) United States
(12) Patent Application Publication
(10) Pub. No.: US 2017/0145005 A1
(43) Pub. Date: May 25, 2017

(54) KINASE INHIBITOR

(71) Applicants: THE UNIVERSITY OF TOKYO, Tokyo (JP); NATIONAL UNIVERSITY CORPORATION KANAZAWA UNIVERSITY, Kanazawa-shi, Ishikawa (JP)

(72) Inventors: Tetsuo NAGANO, Tokyo (JP); Hirofumi NAKANO, Tokyo (JP); Tsukasa HASEGAWA, Tokyo (JP); Nae SAITO, Tokyo (JP); Hirotatsu KOJIMA, Tokyo (JP); Takayoshi OKABE, Tokyo (JP); Naofumi MUKAIDA, Ishikawa (JP)

(73) Assignee: THE UNIVERSITY OF TOKYO, Tokyo (JP)

(21) Appl. No.: 15/116,955

(22) PCT Filed: Feb. 13, 2015
(86) PCT No.: PCT/JP2015/054028

§ 371 Date: Aug. 5, 2016

(30) Foreign Application Priority Data
Feb. 13, 2014 (JP) 2014-025781

(51) Int. Cl. C07D 471/04; C07D 519/00

(52) U.S. Cl. CPC C07D 471/04; C07D 519/00; C07B 2200/07

(57) ABSTRACT

[Problem]

To provide a novel PIM-3 inhibitor and a novel cancer therapeutic drug, in particular, a therapeutic drug for pancreatic cancer.

[Solution]

A PIM-3 kinase inhibitor comprising a compound represented by general formula (I) or a pharmacologically acceptable salt, hydrate or solvate thereof.

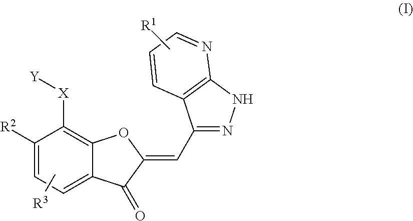

### TECHNICAL FIELD

The present invention relates to an anticancer agent containing as an active ingredient a pyrazolo[3,4-b]pyridine compound having a potent inhibitory effect on PIM-3 kinase, or a pharmacologically acceptable salt thereof.

### BACKGROUND ART

PIM-3 is one of the PIM kinase family, and is a serine/threonine kinase. The PIM kinase family, which comprises PIM-1, -2, and -3, phosphorylate proteins associated with apoptosis and cell cycle control and promote the oncogenic transformation of cells and cancer cell growth (Non-patent Reference 1). PIM-3 as well is reported to phosphorylate Bad, which is a proapoptotic protein, and suppress apoptosis of cells (Non-patent Reference 2). The expression of PIM-1 and PIM-2 is enhanced in certain types of leukemia and prostate cancer, but it is characteristic of PIM-3 that its expression is enhanced in pancreatic cancer, liver cancer, colon cancer, stomach cancer, and other such endoderm-derived organ cancers (Non-patent Reference 2). Based on these reports, PIM-3 inhibitors are expected to be useful as cancer therapeutics, especially as therapeutics for endoderm-derived organ cancers.

Several PIM-3 inhibitors have been reported in recent years (Non-patent Reference 1), for example, phenanthrene compounds (Patent Reference 1, Non-patent Reference 3). Other examples include AstraZeneca's AZD-1208 as a compound having a significant PIM-3 inhibitory effect (Patent Reference 2, Non-patent Reference 4). Even though the development of PIM-3 inhibitors is being advanced in this way, none has yet been marketed, and there is a demand for the development of new drugs having superior activity.

The PIM-1 inhibitory activity of pyrazolo[3,4-b]pyridine compounds is described in International Publication 2011/136319, but no PIM-3 inhibitory activity is reported. As for inhibitory activity on cancer cell growth, growth-inhibiting activity is reported in cancer cells derived from leukemia, but there is no report of an effect on solid cancers such as pancreatic cancer (Patent Reference 3).

### PRIOR ART REFERENCES

### Patent References

Patent Reference 1: International Publication 2012/164969
Patent Reference 2: International Publication 2010/001169
Patent Reference 3: International Publication 2011/136319

### Non-Patent References

Non-Patent Reference 1: Medicinal Research Reviews, 34, pp. 136-159, 2014.
Non-Patent Reference 2: Cancer Science, 102, pp. 1437-1442, 2011.
Non-Patent Reference 3: Cancer Science, 103, pp. 107-115, 2012.
Non-Patent Reference 4: Bioorganic & Medicinal Chemistry Letters, 22, pp. 4599-4604, 2012.

### SUMMARY OF THE INVENTION

### Problems to be Solved by the Invention

The purpose of the present invention is to provide a novel PIM-3 inhibitor, a novel therapeutic for endoderm-derived organ cancers, and in particular a therapeutic for pancreatic cancer.

### Means Used to Solve the Above-Mentioned Problems

When the present inventors conducted in-depth studies to solve the above problems, they discovered that compounds having a specific structure among pyrazolo[3,4-b]pyridine compounds have a potent inhibitory effect on PIM-3 kinase and thereby perfected the present invention.

Specifically, the present invention is

[1] a PIM-3 kinase inhibitor containing a compound represented by the following general formula (I):

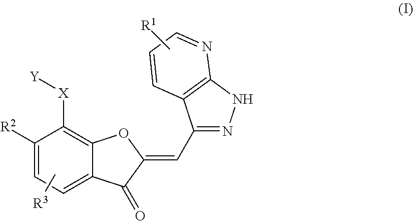

(wherein

R¹ represents a hydrogen atom or from one to three identical or different substituents on the pyridine ring;

R² represents a hydrogen atom, halogen, hydroxyl group, C₁₋₆ alkoxy group, halo-substituted C₁₋₆ alkoxy group, aryl-substituted C₁₋₆ alkoxy group, aryloxy-substituted C₁₋₆ alkoxy group, hydroxy-substituted C₁₋₆ alkoxy group, or C₁₋₆ alkoxy-substituted C₁₋₆ alkoxy group;

R³ represents a hydrogen atom or one or two identical or different substituents on the benzene ring;

X is a methylene group or ethylene group; the methylene group or ethylene group may be substituted by from one to four C₁₋₄ alkyl groups or C₁₋₄ alkylene groups;

Y represents a substituted or unsubstituted heterocyclic group)

or a pharmacologically acceptable salt, hydrate, or solvate thereof.

[2] The PIM-3 kinase inhibitor according to [1], wherein Y is a substituted or unsubstituted piperidine ring group, piperazine ring group, morpholine ring group, or pyrrolidine ring group.

[3] The PIM-3 kinase inhibitor according to [1] or [2], wherein Y is a heterocyclic group substituted by at least one substituent selected from the group consisting of a halogen, hydroxyl group, C₁₋₆ alkyl group, amino group, and amino-substituted C₁₋₆ alkyl group, and wherein, if the heterocyclic group is substituted by two or more C₁₋₆ alkyl groups, some of these alkyl groups may bond to each other to form a ring.

[4] The PIM-3 kinase inhibitor according to any of [1]-[3], wherein R² is selected from a hydrogen atom, halogen, hydroxyl group, C₁₋₆ alkoxy group, halo-substituted C₁₋₆ alkoxy group, hydroxy-substituted C₁₋₆ alkoxy group, or C₁₋₆ alkoxy-substituted C₁₋₆ alkoxy group.

[5] A pharmaceutical composition for treating or preventing endoderm-derived organ cancers containing a compound represented by the following general formula (I):

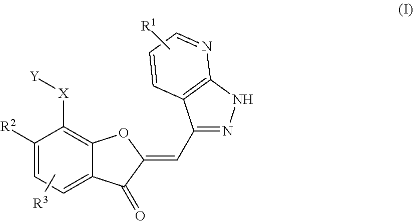

(wherein

R¹ represents a hydrogen atom or from one to three identical or different substituents on the pyridine ring;

R² represents a hydrogen atom, halogen, hydroxyl group, C₁₋₆ alkoxy group, aryl-substituted C₁₋₆ alkoxy group, aryloxy-substituted C₁₋₆ alkoxy group, hydroxy-substituted C₁₋₆ alkoxy group, or C₁₋₆ alkoxy-substituted C₁₋₆ alkoxy group;

R³ represents a hydrogen atom or one or two identical or different substituents the a benzene ring;

X is a methylene group or ethylene group; the methylene group or ethylene group may be substituted by from one to four C₁₋₄ alkyl groups or C₁₋₄ alkylene groups;

Y represents a substituted or unsubstituted heterocyclic group)

or a pharmacologically acceptable salt, hydrate, or solvate thereof.

[6] The pharmaceutical composition according to [5], wherein the endoderm-derived organ cancer is pancreatic cancer.

[7] A compound represented by the following general formula (I):

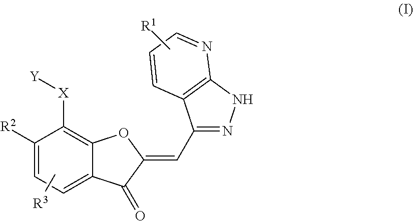

(wherein

R¹ represents a hydrogen atom or from one to three identical or different substituents on the pyridine ring;

R² represents a hydrogen atom, halogen, hydroxyl group, C₁₋₆ alkoxy group, halo-substituted C₁₋₆ alkoxy group, aryl-substituted C₁₋₆ alkoxy group, aryloxy-substituted C₁₋₆ alkoxy group, hydroxy-substituted C₁₋₆ alkoxy group, or C₁₋₆ alkoxy-substituted C₁₋₆ alkoxy group;

R³ represents a hydrogen atom or one or two identical or different substituents on the benzene ring;

X is a methylene group or ethylene group; the methylene group or ethylene group may be substituted by from one to four C₁₋₄ alkyl groups or C₁₋₄ alkylene groups;

Y represents a substituted or unsubstituted heterocyclic group)

or a salt thereof with the proviso that the following compounds are excluded.

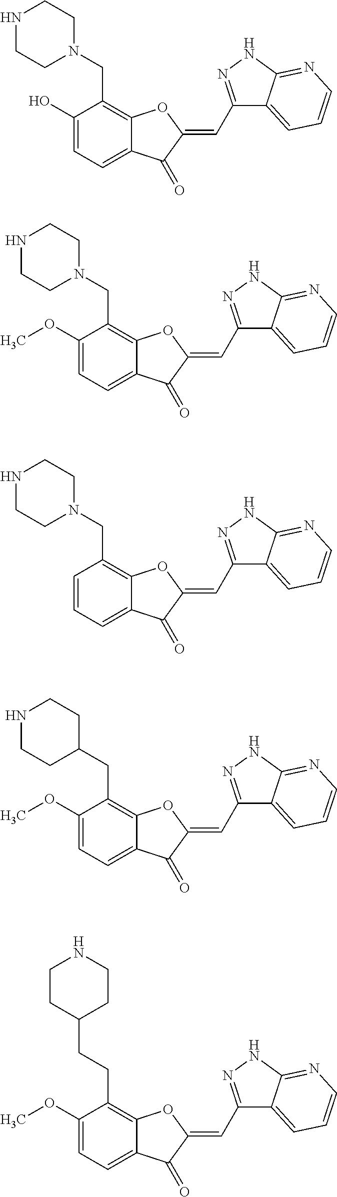 [SMILES: O=C1/C(=C/c2n[nH]c3ncccc23)Oc2c1ccc(O)c2CN1CCNCC1 ; COc1ccc2c(c1CN1CCNCC1)O/C(=C\c1n[nH]c3ncccc13)C2=O ; O=C1/C(=C/c2n[nH]c3ncccc23)Oc2c(CN3CCNCC3)cccc21 ; COc1ccc2c(c1CC1CCNCC1)O/C(=C\c1n[nH]c3ncccc13)C2=O ; COc1ccc2c(c1CCC1CCNCC1)O/C(=C\c1n[nH]c3ncccc13)C2=O]

[8] The compound according to [7], wherein Y is a substituted or unsubstituted piperidine ring group, piperazine ring group, morpholine ring group, or pyrrolidine ring group.

[9] The compound according to [7] or [8], wherein Y is a heterocyclic group substituted by at least one substituent selected from the group consisting of a halogen, hydroxyl group, C₁₋₆ alkyl group, amino group, and amino-substituted C₁₋₆ alkyl group, and wherein, if the heterocyclic group is substituted by two or more C₁₋₆ alkyl groups, some of these alkyl groups may bond to each other to form a ring.

[10] The compound according to [7], wherein Y is represented by the following formula (1), (2), or (3):

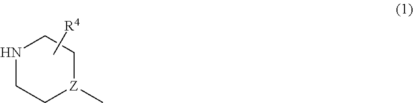

(wherein R⁴ represents a hydrogen atom or from one to nine identical or different substituents on the six-membered ring; Z represents a carbon or nitrogen)

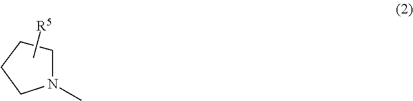

(wherein R⁵ represents a hydrogen atom or from one to eight identical or different substituents on the pyrrolidine ring)

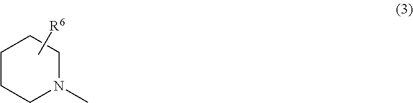

(wherein R⁶ represents a hydrogen atom or from one to ten identical or different substituents on the piperidine ring).

[11] The compound according to [10], wherein R⁴ is selected from the group consisting of a hydrogen atom, halogen, C₁₋₆ alkyl group, amino group, and amino-substituted C₁₋₆ alkyl group, and wherein, if R⁴ is two or more C₁₋₆ alkyl groups, some of these alkyl groups may bond to each other to form a ring.

[12] The compound according to [10], wherein R⁵ is selected from the group consisting of a hydrogen atom, halogen, C₁₋₆ alkyl group, amino group, and amino-substituted C₁₋₆ alkyl group, and wherein, if R⁵ is two or more C₁₋₆ alkyl groups, some of these alkyl groups may bond to each other to from a ring, with the proviso that at least one R⁵ is an amino group and amino-substituted C₁₋₆ alkyl group.

[13] The compound according to [10], wherein R⁶ is selected from the group consisting of a hydrogen atom, halogen, C₁₋₆ alkyl group, amino group, and amino-substituted C₁₋₆ alkyl group, and wherein, if R⁶ is two or more C₁₋₆ alkyl groups, some of these alkyl groups may bond to each other to form a ring.

[14] The compound according to any of [7]-[13], wherein R² is selected from a hydrogen atom, halogen, hydroxyl group, C₁₋₆ alkoxy group, halo-substituted C₁₋₆ alkoxy group, hydroxy-substituted C₁₋₆ alkoxy group, or C₁₋₆ alkoxy-substituted C₁₋₆ alkoxy group.

[15] Use of a compound represented by the following general formula (I) for the preparation of a medicament for the treatment or prevention of pancreatic cancer.

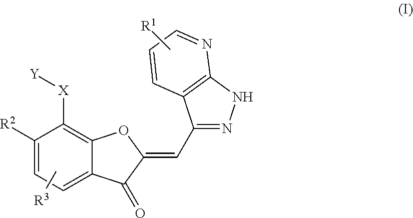

(wherein

R¹ represents a hydrogen atom or from one to three identical or different substituents on the pyridine ring;

R² represents a hydrogen atom, halogen, hydroxyl group, C₁₋₆ alkoxy group, halo-substituted C₁₋₆ alkoxy group, aryl-substituted C₁₋₆ alkoxy group, aryloxy-substituted C₁₋₆ alkoxy group, hydroxy-substituted C₁₋₆ alkoxy group, or C₁₋₆ alkoxy-substituted C₁₋₆ alkoxy group;

R³ represents a hydrogen atom or one or two identical or different substituents on the benzene ring;

X is a methylene group or ethylene group; the methylene group or ethylene group may be substituted by from one to four C₁₋₄ alkyl groups or C₁₋₄ alkylene groups;

Y represents a substituted or unsubstituted heterocyclic group).

### Effect of the Invention

The compound group of the present invention has a potent inhibitory effect on PIM-3 kinase, and has been found to inhibit or suppress the growth of pancreatic cancer cells. The compound group of the present invention is therefore expected to serve as effective therapeutics for pancreatic cancer.

### BRIEF DESCRIPTION OF THE DRAWINGS

FIG. 1 shows the results of an animal study of compound 6 according to the present invention.

### EMBODIMENTS FOR CARRYING OUT THE INVENTION

Embodiments of the present invention are described below. The scope of the present invention is not restricted to these descriptions, it being possible to implement the present invention with suitable variations even outside the following examples as long the spirit of the invention is not impaired.

### 1. DEFINITIONS

In this specification, “halogen atom” means a fluorine atom, chlorine atom, bromine atom, or iodine atom.

In this specification, “alkyl” may be any aliphatic hydrocarbon group having a straight, branched, or cyclic structure, or a combination of these structures. The number of carbon atoms in the alkyl group is not particularly limited, but is, for example, 1-20 (C₁₋₂₀). When the number of carbon atoms is specified, it means an “alkyl” having a number of carbon atoms within that numerical range. For example, C₁₋₈ alkyls include methyl, ethyl, n-propyl, isopropyl, n-butyl, isobutyl, sec-butyl, tert-butyl, n-pentyl, isopentyl, neo-pentyl, n-hexyl, isohexyl, n-heptyl, n-octyl, and the like. In this specification, an alkyl group may have one or more optional substituents. Examples of substituents include, but are not limited to, an alkoxy group, halogen atom, amino group, mono- or di-substituted amino group, substituted silyl group, or acyl, or the like. When an alkyl group has two or more substituents, they may be the same or different. The same is also true for the alkyl moiety of other substituents including an alkyl moiety (for example, an alkoxy group, arylalkyl group, or the like).

In this specification, “alkenyl” means a straight-chain or branched hydrocarbon group having at least one carbon-carbon double bond. Examples include, without limitation, vinyl, allyl, 1-propenyl, isopropenyl, 1-butenyl, 2-butenyl, 3-butenyl, 1,3-butanedienyl, 1-pentenyl, 2-pentenyl, 3-pentenyl, 4-pentenyl, 1,3-pentanedienyl, 1-hexenyl, 2-hexenyl, 3-hexenyl, 4-hexenyl, 5-hexenyl, and 1,4-hexanedienyl. The double bond may have either a cis conformation or trans conformation.

In this specification, “aryl” may be either a monocyclic or fused polycyclic aromatic hydrocarbon group, or an aromatic heterocyclic group including one or more hetero atoms (for example, an oxygen atom, nitrogen atom, or sulfur atom) as ring constituent atoms. In this case, it is also sometimes called “heteroaryl” or “heteroaromatic.” When an aryl is monocyclic or a fused ring, it can bond at all possible positions. Examples of monocyclic aryls include, without limitation, a phenyl group, thienyl group (2- or 3-thienyl group), pyridyl group, furyl group, thiazolyl group, oxazolyl group, pyrazolyl group, 2-pyrazinyl group, pyrimidinyl group, pyrrolyl group, imidazolyl group, pyridazinyl group, 3-isothiazolyl group, 3-isoxazolyl group, 1,2,4-oxadiazol-5-yl group, or 1,2,4-oxadiazol-3-yl group. Non-limiting examples of fused polycyclic aryls include a 1-naphthyl group, 2-naphthyl group, 1-indenyl group, 2-indenyl group, 2,3-dihydroinden-1-yl group, 2,3-dihydroinden-2-yl group, 2-anthryl group, indazolyl group, quinolyl group, isoquinolyl group, 1,2-dihydroisoquinolyl group, 1,2,3,4-tetrahydroisoquinolyl group, indolyl group, isoindolyl group, phthalazinyl group, quinoxalinyl group, benzofuranyl group, 2,3-dihydrobenzofuran-1-yl group, 2,3-dihydrobenzofuran-2-yl group, 2,3-dihydrobenzothiophen-1-yl group, 2,3-dihydrobenzothiophen-2-yl group, benzothiazolyl group, benzimidazolyl group, fluorenyl group, or thioxanthenyl group. In this specification, an aryl group may have one or more optional substituents on the ring. Examples of these substituents include, but are not limited to, an alkoxy group, halogen atom, amino group, mono- or di-substituted amino group, substituted silyl group, or acyl group. When an aryl group has two or more substituents, they may be the same or different. The same is also true for the aryl moiety of other substituents including an aryl moiety (for example, an aryl-substituted alkoxy group, aryloxy-substituted alkoxy group, or the like).

In this specification, “arylalkyl” represents an alkyl substituted by the above aryl. An arylalkyl may have one or more optional substituents. Examples of these substituents include, without limitation, an alkoxy group, halogen atom, amino group, mono- or di-substituted amino group, substituted silyl group, or acyl group. When an acyl group has two or more substituents, they may be the same or different. Non-limiting examples of arylalkyls include a benzyl group, 2-thienylmethyl group, 3-thienylmethyl group, 2-pyridylmethyl group, 3-pyridylmethyl group, 4-pyridylmethyl group, 2-furylmethyl group, 3-furylmethyl group, 2-thiazolylmethyl group, 4-thiazolylmethyl group, 5-thiazolylmethyl group, 2-oxazolylmethyl group, 4-oxazolylmethyl group, 5-oxazolylmethyl group, 1-pyrazolylmethyl group, 3-pyrazolylmethyl group, 4-pyrazolylmethyl group, 2-pyrazinylmethyl group, 2-pyrimidinylmethyl group, 4-pyrimidinylmethyl group, 5-pyrimidinylmethyl group, 1-pyrrolylmethyl group, 2-pyrrolylmethyl group, 3-pyrrolylmethyl group, 1-imidazolylmethyl group, 2-imidazolylmethyl group, 4-imidazolylmethyl group, 3-pyridazinylmethyl group, 4-pyridazinylmethyl group, 3-isothiazolylmethyl group, 3-isoxazolylmethyl group, 1,2,4-oxadiazol-5-ylmethyl group, or 1,2,4-oxadiazol-3-ylmethyl group.

Similarly, in this specification, “arylalkenyl” represents an alkenyl substituted by the above aryl.

In this specification, “alkoxy group” is a structure in which the above alkyl group is bonded to an oxygen atom. Examples include saturated alkoxy groups having a straight-chain, branched, or cyclic structure, or a combination thereof. For example, a methoxy group, ethoxy group, n-propoxy group, isopropoxy group, cyclopropoxy group, n-butoxy group, isobutoxy group, s-butoxy group, t-butoxy group, cyclobutoxy group, cyclopropylmethoxy group, n-pentyloxy group, cyclopentyloxy group, cyclopropylethyloxy group, cyclobutylmethyloxy group, n-hexyloxy group, cyclohexyloxy group, cyclopropylpropyloxy group, cyclobutylethyloxy group, or cyclopentylmethyloxy group can be given as suitable examples.

In this specification, “aryloxy group” is a group in which the above aryl groups are bonded via oxygen atoms. Examples of aryloxy groups include a phenoxy group, 2-thienyloxy group, 3-thienyloxy group, 2-pyridyloxy group, 3-pyridyloxy group, 4-pyridyloxy group, 2-furyloxy group, 3-furyloxy group, 2-thiazolyloxy group, 4-thiazolyloxy group, 5-thiazolyloxy group, 2-oxazolyloxy group, 4-oxazolyloxy group, 5-oxazolyloxy group, 1-pyrazolyloxy group, 3-pyrazolyloxy group, 4-pyrazolyloxy group, 2-pyrazinyloxy group, 2-pyrimidinyloxy group, 4-pyrimidinyloxy group, 5-pyrimidinyloxy group, 1-pyrrolyloxy group, 2-pyrrolyloxy group, 3-pyrrolyloxy group, 1-imidazolyloxy group, 2-imidazolyloxy group, 4-imidazolyloxy group, 3-pyridazinyloxy group, 4-pyridazinyloxy group, 3-isothiazolyloxy group, 3-isoxazolyloxy group, 1,2,4-oxadiazol-5-yloxy group, or 1,2,4-oxadiazol-3-yl group.

In this specification, “alkylene” is a divalent group composed of a straight-chain or branched saturated hydrocarbon, and is represented by —CₙH₂ₙ— or ═CₙH₂ₙ. Examples include methylene (forms having a double bond are also referred to as methylidene), 1-methylmethylene, 1,1-dimethylmethylene, ethylene (forms having a double bond are also referred to as ethylidene), 1-methylethylene, 1-ethylethylene, 1,1-dimethylethylene, 1,2-dimethylethylene, 1,1-diethylethylene, 1,2-diethylethylene, 1-ethyl-2-methylethylene, trimethylene, 1-methyltrimethylene, 2-methyltrimethylene, 1,1-dimethyltrimethylene, 1,2-dimethyltrimethylene, 2,2-dimethyltrimethylene, 1-ethyltrimethylene, 2-ethyltrimethylene, 1,1-diethyltrimethylene, 1,2-diethyltrimethylene, 2,2-diethyltrimethylene, 2-ethyl-2-methyltrimethylene, tetramethylene, 1-methyltetramethylene, 2-methyltetramethylene, 1,1-dimethyltetramethylene, 1,2-dimethyltetramethylene, 2,2-dimethyltetramethylene, 2,2-di-n-propyltrimethylene, and the like.

In this specification, “alkenylene” is a divalent group composed of a straight-chain or branched unsaturated hydrocarbon having at least one carbon-carbon double bond. Examples include ethenylene, 1-methylethenylene, 1-ethylethenylene, 1,2-dimethylethenylene, 1,2-diethylethenylene, 1-ethyl-2-methylethenylene, propenylene, 1-methyl-2-propenylene, 2-methyl-2-propenylene, 1,1-dimethyl-2-propenylene, 1,2-dimethyl-2-propenylene, 1-ethyl-2-propenylene, 2-ethyl-2-propenylene, 1,1-diethyl-2-propenylene, 1,2-diethyl-2-propenylene, 1-butenylene, 2-butenylene, 1-methyl-2-butenylene, 2-methyl-2-butenylene, 1,1-dimethyl-2-butenylene, 1,2-dimethyl-2-butenylene, and the like.

In this specification, “arylene” and “arylalkylene” mean divalent groups based on the above “aryl” and “arylalkyl,” respectively. Similarly, “oxyalkylene” and “aryleneoxy” mean divalent groups based on the above “alkoxy” and “aryloxy,” respectively.

In this specification, “alkylamino” and “arylamino” mean amino groups in which hydrogen atoms of the —NH₂ group have been substituted by one or two of the above alkyls or aryls. Examples include methylamino, dimethylamino, ethylamino, diethylamino, ethylmethylamino, benzylamino, and the like. Similarly, “alkylthio” and “arylthio” mean groups in which hydrogen atoms of the —SH group have been substituted by the above alkyls or aryls. Examples include methylthio, ethylthio, benzylthio, and the like.

In this specification, the phrase “ring structure” means a heterocyclic or carbocyclic group when formed by a combination of two substituents. Such groups may be saturated, unsaturated, or aromatic. Examples include cycloalkyl, phenyl, naphthyl, morpholinyl, piperidinyl, imidazolyl, pyrrolidinyl, and pyridyl. In this specification, substituents can form ring structures with other substituents, and those skilled in the art can understand that a specific substitution, for example, bonding to hydrogen, is formed when such substituents bond to each other. Therefore, when it is stated that specific substituents together form a ring structure, those skilled in the art can understand that this ring structure can be formed by an ordinary chemical reaction or is generated easily. Any such ring structures and their formation process are within the purview of those skilled in the art.

### 2. COMPOUNDS ACCORDING TO THE PRESENT INVENTION

One embodiment of the present invention is a compound represented by the following general formula (I):

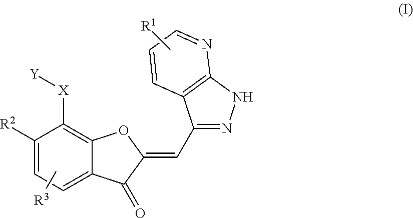

or the salt thereof, with the proviso that the following compounds are excluded.

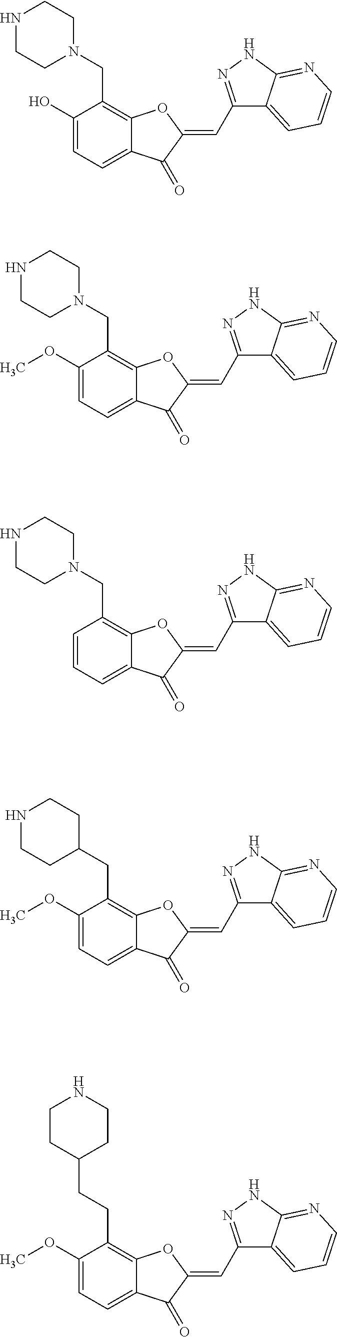

In formula (I), R¹ represents a hydrogen atom or from one to three substituents on the pyridine ring. These substituents are preferably selected from the group consisting of a halogen atom, C₁₋₆ alkyl group, C₂₋₆ alkenyl group, C₂₋₆ alkynyl group, halo-substituted C₁₋₆ alkyl group, hydroxy-substituted C₁₋₆ alkyl group, amino-substituted C₁₋₆ alkyl group, hydroxyl group, C₁₋₆ alkoxy group, halo-substituted C₁₋₆ alkoxy group, C₁₋₆ alkoxycarbonyl group, amino group, nitro group, aryl group, aralkyloxy group, heterocyclic group, and heterocyclic group-substituted C₁₋₆ alkoxy group.

Each R¹ present on the pyridine ring may be the same or different.

In formula (I), R² represents a hydrogen atom, halogen, hydroxyl group, C₁₋₆ alkoxy group, halo-substituted C₁₋₆ alkoxy group, aryl-substituted C₁₋₆ alkoxy group, aryloxy-substituted C₁₋₆ alkoxy group, hydroxy-substituted C₁₋₆ alkoxy group, or C₁₋₆ alkoxy-substituted C₁₋₆ alkoxy group.

In formula (I), R³ shows a hydrogen atom or one or two substituents on the benzene ring. These substituents are preferably selected from a halogen atom, C₁₋₆ alkyl group, C₁₋₆ alkoxy group, aryl group, amino group, hydroxyl group, or heterocyclic group.

Each R³ present on the benzene ring may be the same or different.

In formula (I), X represents a methylene group or ethylene group. The methylene group or ethylene group may be substituted by from one to four C₁₋₄ alkyl groups or C₁₋₄ alkylene groups (represented by ═CₙH₂ₙ (n is an integer of 1-4)). A methyl group and methylene group (═CH₂ group) are preferred as a C₁₋₄ alkyl group or C₁₋₄ alkylene group.

In formula (I), Y represents a substituted or unsubstituted heterocyclic group. A substituted or unsubstituted piperidine ring group, piperazine ring group, morpholine ring group, or pyrrolidine ring group is preferred as a substituted or unsubstituted heterocyclic group.

In one preferred embodiment of the present invention, Y in formula (I) is a heterocyclic group substituted by at least one substituent selected from the group consisting of a halogen, C₁₋₆ alkyl group, amino group, and amino-substituted C₁₋₆ alkyl group. Here, when the heterocyclic group is substituted by two or more C₁₋₆ alkyl groups, some of these alkyl groups (preferably two C₁₋₆ alkyl groups) may bond to each other and form a ring.

In one aspect of the present invention, Y in formula (I) is represented by the following formula (1), (2), or (3):

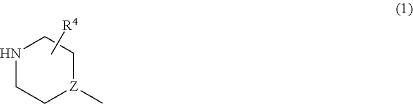

(wherein R⁴ represents a hydrogen atom or one to eight (when Z is nitrogen) or one to nine (when Z is carbon) identical or different substituents substituted on a six-membered ring; Z represents carbon or nitrogen)

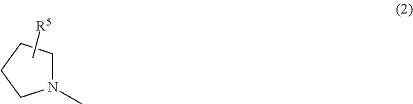

(wherein R⁵ represents a hydrogen atom or one to eight identical or different substituents on the pyrrolidine ring)

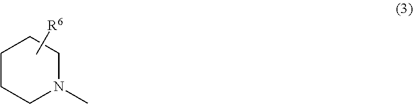

(wherein R⁶ represents a hydrogen atom or one to ten identical or different substituents on the piperidine ring).

In one preferred aspect of the present invention, R⁴ in formula (1) is selected from the group consisting of a hydrogen atom, halogen, C₁₋₆ alkyl group, amino group, and amino-substituted C₁₋₆ alkyl group. Here, when R⁴ is two or more C₁₋₆ alkyl groups, some of these alkyl groups (preferably two C₁₋₆ alkyl groups) may bond to each other and form a ring. R⁴ is preferably selected from a hydrogen atom, halogen, or C₁₋₆ alkyl group; particularly preferably a hydrogen atom, methyl group, or fluorine.

In one preferred embodiment of the present invention, Z is carbon, eight R⁴ are hydrogen atoms, and one R⁴ is fluorine.

In one preferred aspect of the present invention, R⁵ in formula (2) is selected from the group consisting of a hydrogen atom, halogen, C₁₋₆ alkyl group, amino group, and amino-substituted C₁₋₆ alkyl group. Here, when R⁵ is two or more C₁₋₆ alkyl groups, some of these alkyl groups (preferably two C₁₋₆ alkyl groups) may bond to each other and form a ring. Preferably, at least one R⁵ is selected from an amino group or amino-substituted C₁₋₆ alkyl group.

In one preferred embodiment of the present invention, at least one R⁵ is an amino group or amino-substituted C₁₋₆ alkyl group, and the remainder are hydrogen atoms.

In one preferred aspect of the present invention, R⁶ in formula (3) is selected from the group consisting of a hydrogen atom, halogen, C₁₋₆ alkyl group, amino group, and amino-substituted C₁₋₆ alkyl group. Here, when R⁶ is two or more C₁₋₆ alkyl groups, some of these alkyl groups (preferably two C₁₋₆ alkyl groups) may bond to each other and form a ring. Preferably, at least one R⁶ is selected from an amino group or amino-substituted C₁₋₆ alkyl group.

In one preferred embodiment of the present invention, at least one R⁶ is an amino group or amino-substituted C₁₋₆ alkyl group, and the remainder are hydrogen atoms.

The compounds of the present invention, unless specifically stated otherwise, also include their stereoisomers such as tautomers, geometric isomers (for example, E-form, Z-form, and the like), and enantiomers. Specifically, when a compound represented by formula (1) includes one or more asymmetrical carbons, it can take either an (R)-form or (S)-form, each independently, in accordance with the stereochemistry of asymmetrical carbons, and sometimes exists as an enantiomer or diastereomer or other such stereoisomer of that derivative.

The following compounds can be given as non-limiting concrete examples of compounds represented by formula (I).
(Z)-2-[(1H-pyrazolo[3,4-b]pyridin-3-yl)methylene]-6-methoxy-7-[1-(piperazin-1-yl)ethyl]benzofuran-3(2H)-one (compound 6)
(Z)-6-methoxy-2-[(6-methyl-1H-pyrazolo[3,4-b]pyridin-3-yl)methylene]7-(piperazin-1-ylmethyl)benzofuran-3(2H)-one (compound 7)
(R,Z)-2-[(1H-pyrazolo[3,4-b]pyridin-3-yl)methylene]-6-methoxy-7-[(2-methylpiperazin-1-yl)methyl]benzofuran-3(2H)-one (compound 8)
(S,Z)-2-[(1H-pyrazolo[3,4-b]pyridin-3-yl)methylene]-6-methoxy-7-[(2-methylpiperazin-1-yl)methyl]benzofuran-3(2H)-one (compound 9)
(Z)-2-[(1H-pyrazolo[3,4-b]pyidin-3-yl)methylene]-7-[(cis-3,5-dimethylpiperazin-1-yl)methyl]-6-methoxybenzofuran-3(2H)-one (compound 10)
(S,Z)-2-[(1H-pyrazolo[3,4-b]pyridin-3-yl)methylene]-6-methoxy-7-[(3-methylpiperazin-1-yl)methyl]benzofuran-3(2H)-one (compound 11)
(R,Z)-2-[(1H-pyrazolo[3,4-b]pyridin-3-yl)methylene]-6-methoxy-7-[(3-methylpiperazin-1-yl)methyl]benzofuran-3(2H)-one (compound 12)
(Z)-2-[(1H-pyrazolo[3,4-b]pyridin-3-yl)methylene-7-[(cis-2,6-dimethylpiperazin-1-yl)methyl]-6-methoxybenzofuran-3(2H)-one (compound 13)
(S,Z)-2-[(1H-pyrazolo[3,4-b]pyridin-3-yl)methylene]-7-[(3-aminopyrrolidin-1-yl)methyl]-6-methoxybenzofuran-3(2H)-one (compound 14)
(R,Z)-2-[(1H-pyrazolo[3,4-b]pyridin-3-yl)methylene]-7-[(3-aminopyrrolidin-1-yl)methyl]-6-methoxybenzofuran-3(2H)-one (compound 15)
(Z)-6-methoxy-2-[(6-methyl-1H-pyrazolo[3,4-b]pyridin-3-yl)methylene]-7-(piperidin-4-ylmethyl)benzofuran-3(2H)-one (compound 16)
(Z)-2-[(1H-pyrazolo[3,4-b]pyridin-3-yl)methylene]-6-(difluoromethoxy)-7-(piperidin-4-ylmethyl)benzofuran-3(2H)-one (compound 17)
(Z)-2-[(1H-pyrazolo[3,4-b]pyridin-3-yl)methylene]-7-[(3-fluoropiperidin-4-yl)methyl]-6-methoxybenzofuran-3(2H)-one (cis isomer) (compound 18)
(Z)-2-[(1H-pyrazolo[3,4-b]pyridin-3-yl)methylene]-7-[(3-fluoropiperidin-4-yl)methyl]-6-methoxybenzofuran-3(2H)-one (trans isomer) (compound 19)
(Z)-2-[(1H-pyrazolo[3,4-b]pyridin-3-yl)methylene]-6-(2-methoxyethoxy)-7-[1-(piperazin-1-yl)ethyl]benzofuran-3(2H)-one (compound 20)
(Z)-2-[(1H-pyrazolo[3,4-b]pyridin-3-yl)methylene]-6-(2-hydroxyethoxy)-7-[1-(piperazin-1-yl)ethyl]benzofuran-3(2H)-one (compound 21)
(Z)-2-[(1H-pyrazolo[3,4-b]pyridin-3-yl)methylene]-6-ethoxy-7-[1-(piperazin-1-yl)ethyl]benzofuran-3(2H)-one (compound 22)
(Z)-2-[(1H-pyrazolo[3,4-b]pyridin-3-yl)methylene]-6-(cyclopropylmethoxy)-7-[1-(piperazin-1-yl)ethyl]benzofuran-3(2H)-one (compound 23)
(Z)-2-[(1H-pyrazolo[3,4-b]pyridin-3-yl)methylene]-6-methoxy-7-[3-(piperazin-1-yl)propa-1-en-2-yl]benzofuran-3(2H)-one (compound 24)
(Z)-2-[(1H-pyrazolo[3,4-b]pyridin-3-yl)methylene]-7-[(3,8-diazabicyclo[3.2.1]octan-3-yl)methyl]-6-methoxybenzofuran-3(2H)-one (compound 25)
(S,Z)-2-[(1H-pyrazolo[3,4-b]pyridin-3-yl)methylene]-6-methoxy-7-{[3-(methylamino)pyrrolidin-1-yl]methyl}benzofuran-3(2H)-one (compound 26)
(Z)-2-[(1H-pyrazolo[3,4-b]pyridin-3-yl)methylene]-6-methoxy-7-[1-(piperazin-1-yl)propan-2-yl]benzofuran-3(2H)-one (compound 27)
(Z)-2-[(1H-pyrazolo[3,4-b]pyridin-3-yl)methylene]-6-chloro-7-(piperidin-4-ylmethyl)benzofuran-3(2H)-one (compound 28)
(Z)-2-[(1H-pyrazolo[3,4-b]pyridin-3-yl)methylene]-6-fluoro-7-(piperidin-4-ylmethyl)benzofuran-3(2H)-one (compound 29)
(R,Z)-2-[(1H-pyrazolo[3,4-b]pyridin-3-yl)methylene]-7-{[2-(aminomethyl)pyrrolidin-1-yl]methyl}-6-methoxybenzofuran-3(2H)-one (compound 31)
(S,Z)-2-[(1H-pyrazolo[3,4-b]pyridin-3-yl)methylene]-7-[(3-aminopiperidin-1-yl)methyl]-6-methoxybenzofuran-3(2H)-one (compound 32)

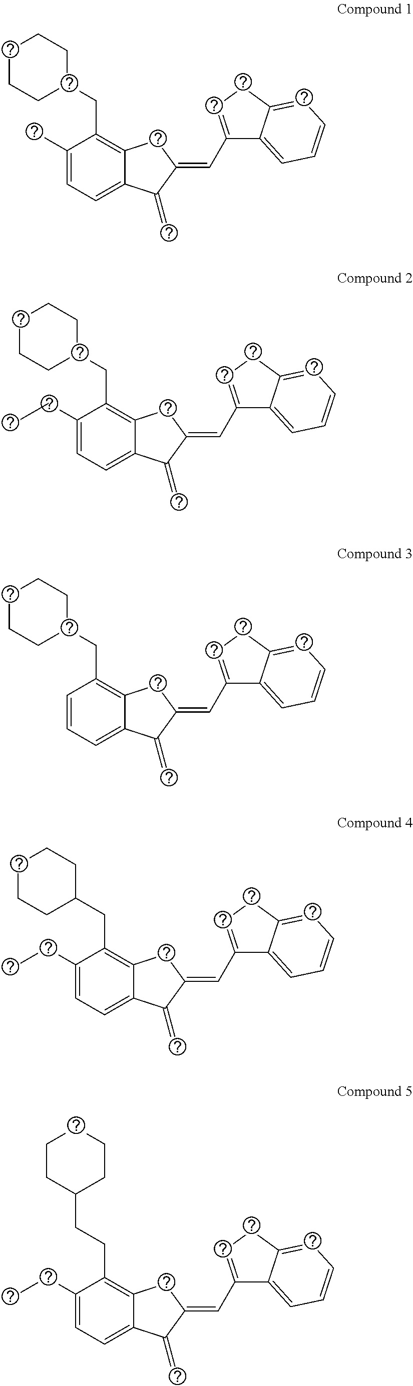

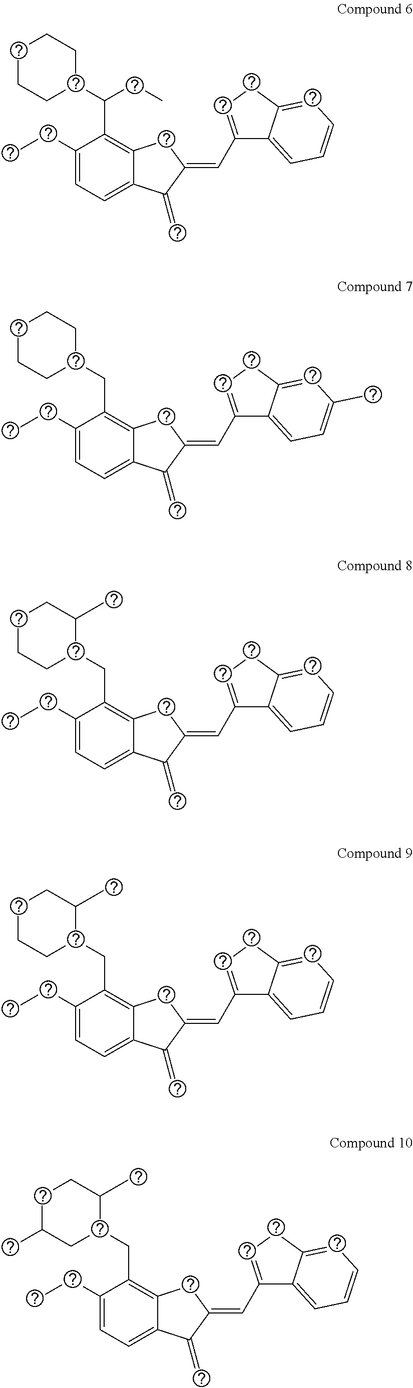

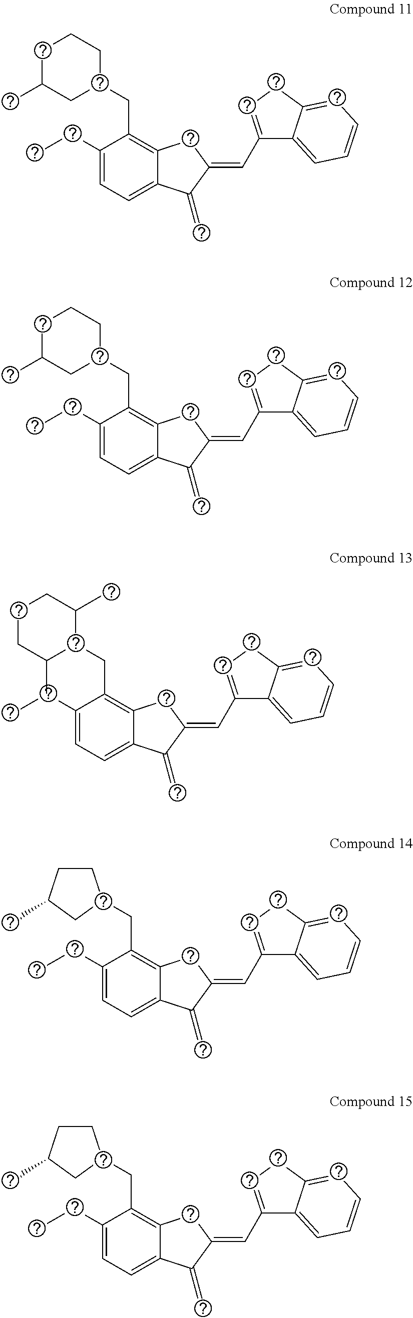

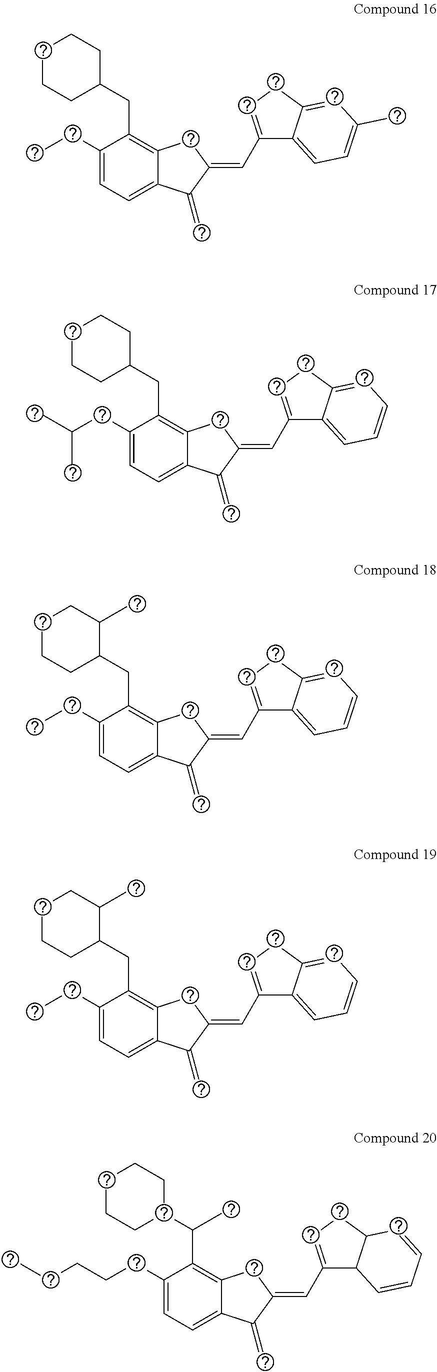

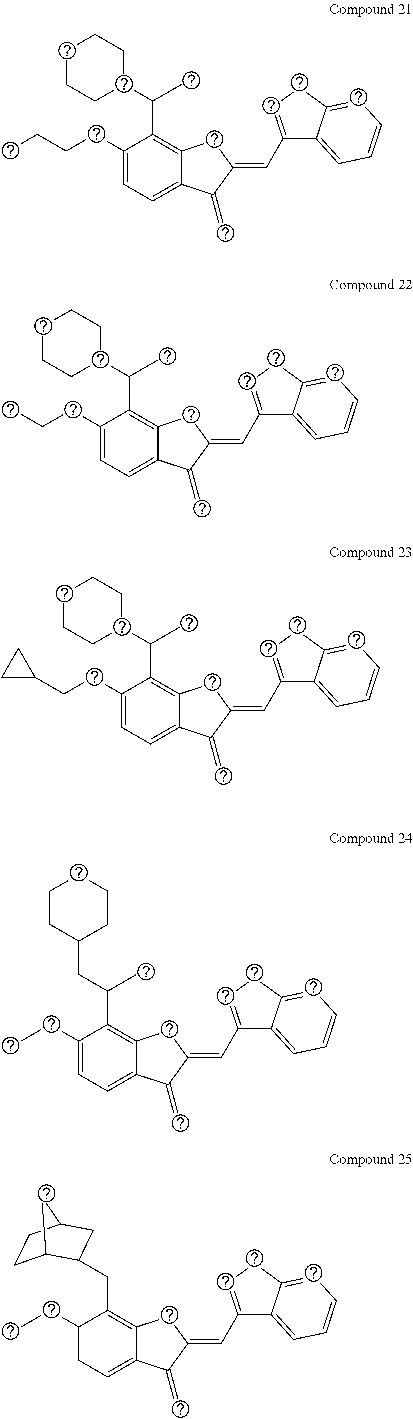

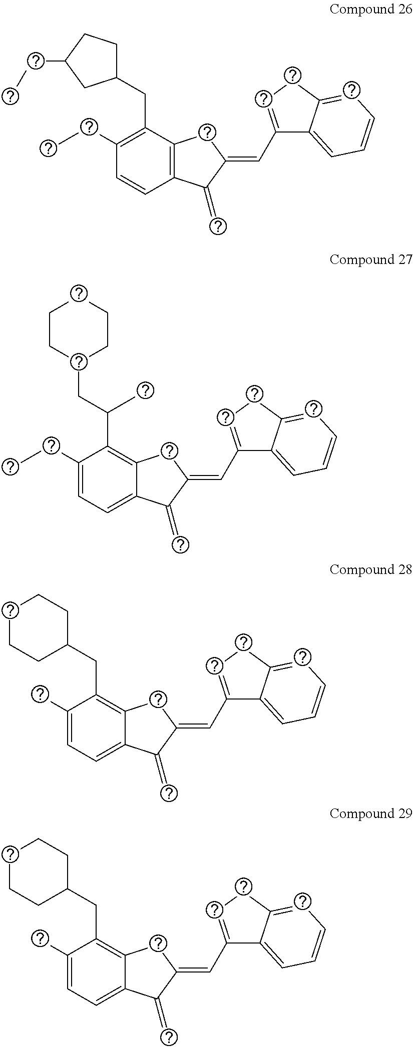

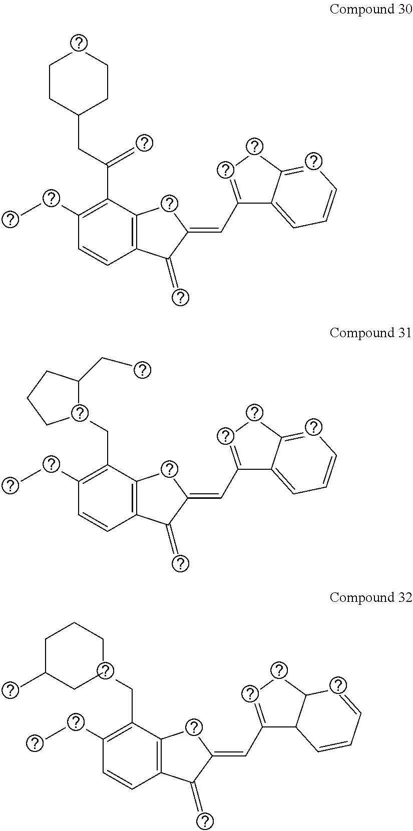

### 3. PIM-3 KINASE INHIBITOR

Another embodiment according to the present invention is a PIM-3 kinase inhibitor containing a compound represented by the following general formula (I):

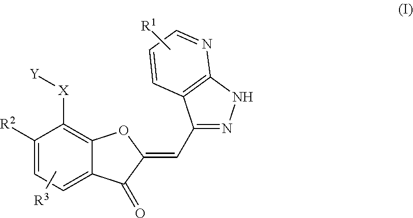

or a pharmacologically acceptable salt, hydrate, or solvate thereof.

In general formula (I), R¹, R², R³, X, and Y are as explained in the above description of the compounds according to the present invention.

In a preferred embodiment of the present invention, Y in formula (I) is a heterocyclic group substituted by a halogen, C₁₋₆ alkyl group, amino group, or amino-substituted C₁₋₆ alkyl group. Here, when the heterocyclic group is substituted by two or more C₁₋₆ alkyl groups, some of these alkyl groups may bond to each other and form a ring.

In one aspect of the present invention, Y in formula (I) is the following formula (1), (2), or (3):

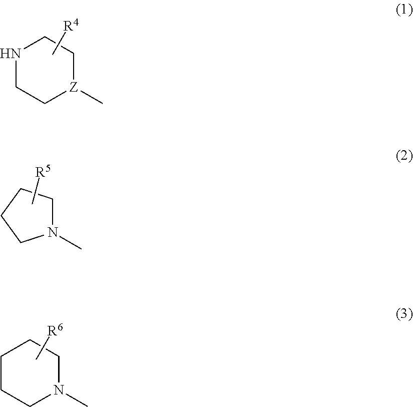

In formula (1), (2), and (3), R⁴, R⁵, and R⁶ are as explained in the above description of compounds of the present invention.

The PIM-3 kinase inhibitor according to the present invention may contain a compound represented by formula (I) or a salt thereof or a solvate or hydrate of these. Salts are not particularly restricted as long as they are pharmacologically acceptable salts. Examples include base addition salts, acid addition salts, amino acid salts, and the like. Examples of base addition salts include a sodium salt, potassium salt, calcium salt, magnesium salt, and other such alkaline earth metal salts; aluminum salt, or triethylamine salt, piperidine salt, morpholine salt, or other such organic amine salts. Examples of acid addition salts include a hydrochloride, hydrobromide, sulfate, nitrate, phosphate, and other such mineral acid salts; methanesulfonic acid, benzenesulfonic acid, p-toluenesulfonic acid, acetic acid, propionic acid, tartaric acid, fumaric acid, maleic acid, malic acid, oxalic acid, succinic acid, citric acid, benzoic acid, mandelic acid, cinnamic acid, lactic acid, glycolic acid, glucuronic acid, ascorbic acid, nicotinic acid, salicylic acid, or other such organic acid salts. Examples of amino acid salts include a glycine salt, aspartate, glutamate, and the like. Aluminum salts and other such metal salts are also acceptable.

The type of solvent that forms a solvate is not particularly restricted. Examples can include solvents such as ethanol, acetone, and isopropanol.

The compounds of the present invention, unless specifically stated otherwise, also include stereoisomers thereof such as tautomers, geometric isomers (for example, E-form, Z-form, and the like), and enantiomers. Specifically, when a compound represented by formula (1) includes one or more asymmetrical carbons, it can take either an (R)-form or (S)-from, each independently, in accordance with the stereochemistry of asymmetrical carbons, and sometimes exists as an enantiomer or diastereomer or other such stereoisomer of that derivative. Therefore, stereoisomers of a pure form, any mixtures of stereoisomers, racemates, and the like are all encompassed within the scope of the present invention as active ingredients of the PIM-3 kinase inhibitor of the present invention.

In addition to a PIM-3 kinase inhibitor containing a compound represented by formula (I) or a pharmacologically acceptable salt, hydrate, or solvate thereof as an active ingredient, the present invention also relates to a pharmaceutical composition for the treatment or prevention of endoderm-derived organ cancers such as pancreatic cancer, liver cancer, colon cancer, and stomach cancer in which expression is enhanced by PIM-3, especially pancreatic cancer (these are collectively referred to as the “drug of the present invention” hereinafter).

A compound represented by general formula (I) or a pharmacologically acceptable salt, hydrate, or solvate thereof, which is the active ingredient, may be administered as the drug of the present invention. However, in general, the drug is preferably administered in the form of a pharmaceutical composition containing the above substance that is the active ingredient and one or more formulation additives. The term “composition” as in a pharmaceutical composition encompasses not only a product containing an active ingredient and an inert ingredient to make up a carrier (pharmacologically acceptable excipient), but also any product produced directly or indirectly as a result of association, complexation, or aggregation of any two or more ingredients, as a result of dissociation of one or more ingredients, or as a result of another type or reaction or interaction of one or more ingredients.

A combination of two or more of the above compounds can be used as the active ingredient of the drug of the present invention, or other active ingredients known to prevent or treat conditions in which expression is enhanced by PIM-3 can also be compounded.

The drug of the present invention can also to made into a combination drug in which a compound represented by general formula (I) or a pharmacologically acceptable salt, hydrate, or solvate thereof which is the active ingredient is used in combination with existing anticancer agents. Those known in the art can be used as existing anticancer agents. Examples include gemcitabine, fluorouracil, oxaliplatin, erlotinib, and the like.

The type of pharmaceutical composition is not particularly restricted. Examples of dosage forms include tablets, capsules, granules, powders, syrups, suspensions, suppositories, ointments, creams, gels, patches, inhalants, injections, and the like. These formulations are prepared by the conventional methods. Furthermore, liquid formulations may be in a form dissolved or suspended in water or another suitable solvent at the time of use. Tablets and granules may also be coated by well-known methods. In the case of an injection, an injection is prepared by dissolving a compound of the present invention in water, but it may be dissolved in physiological saline or glucose solution as needed, or a buffer or preservative may be added. The composition is provided in any dosage form for oral or parenteral administration. For example, a pharmaceutical composition for oral administration can be prepared in the form of granules, fine granules, powder, hard capsules, soft capsules, syrup, emulsion, suspension, or liquid, and a pharmaceutical composition for parenteral administration can be prepared in the form or an injection for intravenous administration, intramuscular administration, subcutaneous administration, or the like, a drip infusion, percutaneously-absorbed agent, transmucosally-absorbed agent, nasal drops, inhalant, suppository, or the like. Injections and drip infusions can also be prepared as a powder in freeze-dried form or the like and used dissolved in a suitable aqueous vehicle such as physiological saline at the time of use. A sustained-release formulation coated by a polymer or the like can also be administered directly into the brain.

The types of formulation additives, proportions of formulation additives to active ingredient, and methods for producing a pharmaceutical composition used in production of the pharmaceutical composition can be selected as is appropriate by one skilled in the art in accordance with the form of the composition. Inorganic or organic materials or solid or liquid materials can be used as formulation additives, and are generally compounded between 1 and 90 wt % relative to the weight of the active ingredient. Concrete examples of such materials include lactose, glucose, mannitol, dextrin, cyclodextrin, starch, sucrose, magnesium aluminometasilicate, synthetic aluminum silicate, carboxymethyl cellulose sodium, hydroxypropyl starch, carboxymethyl cellulose calcium, ion-exchange resin, methyl cellulose, gelatin, gum arabic, hydroxypropyl cellulose, hydroxypropylmethyl cellulose, polyvinyl pyrrolidone, polyvinyl alcohol, light silicic anhydride, magnesium stearate, talc, tragacanth, bentonite, Veegum, titanium oxide, sorbitan fatty acid ester, sodium lauryl sulfate, glycerin, fatty acid glycerin ester, purified lanolin, glycerogelatin, Polysorbate, Macrogol, vegetable oil, wax, liquid paraffin, white petrolatum, fluorocarbon, nonionic surfactant, propylene glycol, water, and the like.

To produce a solid formulation for oral administration, a powder is made by mixing the active ingredient and excipient components, for example, lactose, starch, crystalline cellulose, calcium lactate, silicic anhydride, and the like, and if needed granules are made by adding a binder such as sucrose, hydroxypropyl cellulose, or polyvinyl pyrrolidone, a disintegrating agent such as carboxymethyl cellulose, carboxymethyl cellulose calcium, or the like and conducting wet or dry granulation. To produce tablets, the powder or granules are tableted directly or after adding a lubricant such as magnesium stearate, talc, or the like. The granules or tablets can also be made into an enteric-coated formulation by coating by an enteric coating base such as hydroxypropylmethyl cellulose phthalate or methacrylic acid-methyl methacrylate polymer or into a sustained-release formulation by coating with ethyl cellulose, carnauba wax, hydrogenated oil, or the like. To make capsules, the powder or granules are packed into hard capsules, or the active ingredient can be made into soft capsules as it is or coated by a gelatin film after dissolution in glycerin, polyethylene glycol, sesame oil, olive oil, or the like.

To make an injection, the active ingredient and, as needed, a pH adjuster such as hydrochloric acid, sodium hydroxide, lactose, lactic acid, sodium, sodium monohydrogen phosphate, or sodium dihydrogen phosphate, an isotonifying agent such as sodium chloride, glucose, and the like are dissolved in distilled water for injection, filter sterilized, and packed into ampules. Alternatively, an injection to be dissolved at the time of use may be made by also adding mannitol, dextrin, cyclodextrin, gelatin, or the like and freeze drying. An emulsion for injection can also be made by emulsification in water by adding lecithin, Polysorbate 80, polyoxyethylene hydrogenated castor oil, or the like to the active ingredient.

The dosage and frequency of administration of the drug of the present invention are not particularly restricted and can be selected as is appropriate based on the judgment of a physician in accordance with the goal of treating and/or preventing the malignant transformation or advance of the disease to be treated, the type of disease, the age and weight of the patient, the severity of the disease, and other such conditions. The daily adult dosage in oral administration is generally 0.01-1000 mg (active ingredient weight), which can be administered once a day or divided over several doses or administered every several days. When used as an injection, the daily adult dosage is preferably 0.001-100 mg (active ingredient weight) administered continuously or intermittently.

The method for producing compounds represented by general formula (I) is not particularly restricted, but synthesis methods for novel compounds among typical compounds encompassed by general formula (I) are illustrated concretely in the examples in this specification. Compounds encompassed by general formula (I) can be produced by one skilled in the art by suitably changing or modifying the starting raw materials, reagents, reaction conditions, and the like as is appropriate using the examples and following schemes of this specification as a reference.

### EXAMPLES

The present invention is explained in greater detail below through examples. However, the present invention is not limited by these examples. Unless specifically stated otherwise, the reaction was carried out in an inert gas (nitrogen or argon) atmosphere.

In addition, in the synthesis schemes shown in the examples, Me means a methyl group, Et means an ethyl group, Pr means a propyl group, c-Pr means a cyclopropyl group, Bu means a butyl group, Ac means an acetyl group, Boc means a tert-butoxycarbonyl group, Ph means a phenyl group, TFA means trifluoroacetic acid, TMS means a trimethylsilyl group, 9-BBN means 9-borabicyclo[3.3.1]nonane, THF means tetrahydrofuran, DMF means dimethylformamide, DMSO means dimethyl sulfoxide, DIBAL-H means diisobutylaluminum hydride, DEAD means diethyl azodicarboxylate, NBS means N-bromosuccinimide, BPO means benzoyl peroxide, and rt means room temperature.

1. Synthesis

Various compounds of formula (I), which are the active ingredient of the present invention, were synthesized as follows.

### Synthesis Example 1

### Synthesis of compound 6 ((Z)-2-[(1H-pyrazolo[3,4-b]pyridin-3-yl)methylene]-6-methoxy-7-[1-(piperazin-1-yl)ethyl]benzofuran-3(2H)-one))

Compound 6 was synthesized by synthesis scheme 1 below.

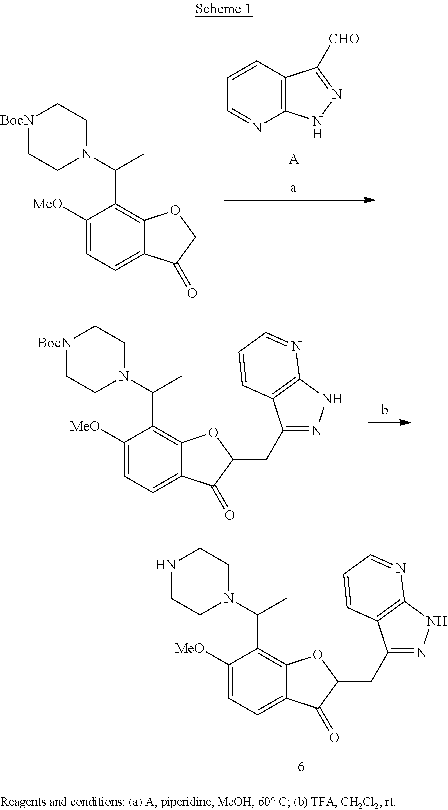

(a) Step 1

1H-pyrazolo[3,4-b]pyridine-3-carbaldehyde (0.0391 g, 0.266 mmol), described in the known literature [WO2011/136319], was added to 1 mL of a methanol solution of tert-butyl 4-[1-(6-methoxy-3-oxo-2,3-dihydrobenzofuran-7-yl)ethyl]piperazin-1-carboxylate (0.100 g, 0.266 mmol), also described in [WO2011/136319]. After adding piperidine (0.0181 g, 0.213 mmol), the reaction system was stirred for two hours at 60° C., and the reaction solution was cooled to room temperature. The precipitated solid was filtered out, and the target tert-butyl (Z)-4-(1-{2-[(1H-pyrazolo[3,4-b]pyridin-3-yl)methylene]-6-methoxy-3-oxo-2,3-dihydrobenzofuran-7-yl}ethyl)piperazin-1-carboxylate (0.0602 g, 44%) was obtained.

¹H NMR (300 MHz, DMSO-d₆) δ 1.34 (s, 9H), 1.55 (d, J=6.6 Hz, 3H), 2.36-2.39 (m, 2H), 3.31-3.35 (m, 6H), 3.97 (s, 3H), 4.13 (q, J=6.6 Hz, 1H), 7.06-7.08 (m, 2H), 7.36 (dd, J=4.4, 8.0 Hz, 1H), 7.78 (d, J=8.0 Hz, 1H), 8.64 (d, J=4.0 Hz, 1H), 9.31 (d, J=8.0 Hz, 1H), 14.30 (brs, 1H).

(b) Step 2

One milliliter of trifluoroacetic acid was added at room temperature to 2 mL of a methylene chloride solution of tert-butyl (Z)-4-(1-{2-[(1H-pyrazolo[3,4-b]pyridin-3-yl)methylene]-6-methoxy-3-oxo-2,3-dihydrobenzofuran-7-yl}ethyl)piperazin-1-carboxylate (0.0452 g, 0.0894 mmol), and stirred for 12 hours at room temperature. The solution was made basic by adding saturated sodium hydrogen carbonate solution to the residue obtained after distilling off the solvent, and then extracted five times by chloroform. After drying by anhydrous sodium sulfate, the solvent was distilled off to obtain (Z)-2-[(1H-pyrazolo[3,4-b]pyridin-3-yl)methylene]-6-methoxy-7-[1-(piperazin-1-yl)ethyl]benzofuran-3(2H)-one (0.0142 g, 39%).

¹H NMR (300 MHz, DMSO-d₆) δ 1.51 (d, J=6.6 Hz, 3H), 2.34-2.40 (m, 2H), 2.59-2.65 (m, 2H), 2.75-2.78 (m, 4H), 3.97 (s, 3H), 4.08 (q, J=6.6 Hz, 1H), 7.05 (s, 1H), 7.07 (d, J=8.8 Hz, 1H), 7.34 (dd, J=4.4, 8.0 Hz, 1H), 7.77 (d, J=8.8 Hz, 1H), 8.64 (d, J=1.4, 4.4 Hz, 1H), 9.39 (d, J=8.0 Hz, 1H).

### Synthesis Example 2

### Synthesis of compound 7 ((Z)-6-methoxy-2-[(6-methyl-1H-pyrazolo[3,4-b]pyridin-3-yl)methylene]-7-(piperazin-1-ylmethyl)benzofuran-3(2H)-one)

Compound 7 was synthesized by synthesis scheme 2 below.

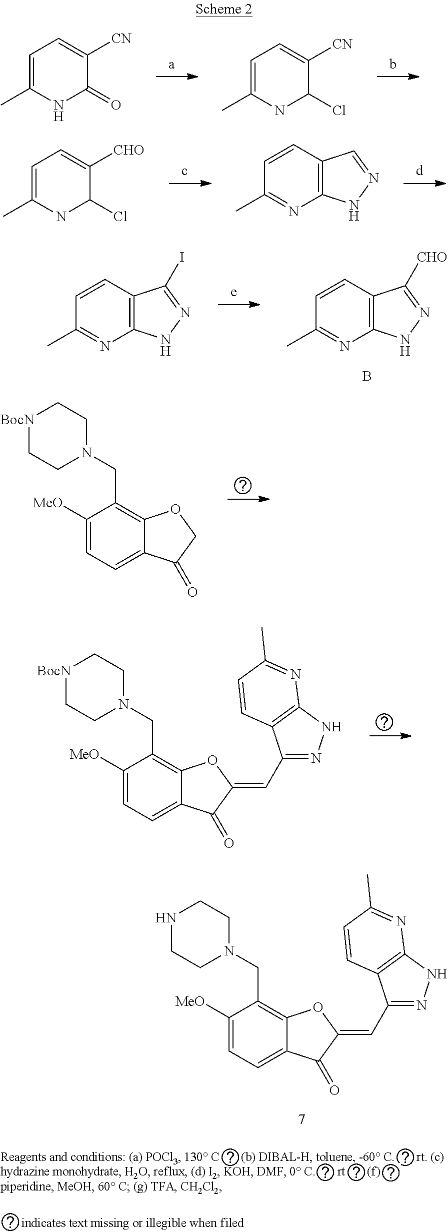

Step 1

6-Methyl-2-oxo-1,2-dihydropyridine-3-carbonitrile (45.0 g, 335 mmol) was added to phosphorus oxychloride (652 g, 4250 mmol), and stirred for two hours at 130° C. The residue obtained by concentrating the reaction solution was dissolved in methylene chloride, and 4 M sodium hydroxide aqueous solution was added until the pH became 8. The organic layer was separated, washed with (saturated) brine, and then dried with anhydrous sodium sulfate. 2-Chloro-6-methylnicotinonitrile (51.0 g, 99%) was obtained by distilling off the solvent under reduced pressure.

¹H NMR (400 MHz, CDCl₃) δ 2.65 (s, 3H), 7.24 (d, J=8.0 Hz, 1H), 7.89 (d, J=8.0 Hz, 1H).

Step 2

Diisobutylaluminum hydride (1 M toluene solution, 242 mL, 242 mmol) was added to 300 mL of a toluene solution of 2-chloro-6-methylnicotinonitrile (35.0 g, 230 mmol) over one hour at −60° C. After stirring for 30 minutes at −60° C., the reaction system was stirred for 1.5 hour at room temperature. A mixed solution of 350 mL of 2 M sulfuric acid aqueous solution and 200 mL of tetrahydrofuran was added dropwise over one hour to the reaction solution that had been cooled to −50° C. After dropwise addition had been completed, the solution was stirred for 18 hours at room temperature, and 200 mL of ethyl acetate was added. The separated organic layer was washed with (saturated) brine, dried by sodium sulfate, and a crude product was obtained by distilling off the solvent.

Step 3

Diisobutylaluminum hydride (1 M toluene solution, 129 mL, 129 mmol) was added to 200 mL of a toluene solution of 2-chloro-6-methylnicotinonitrile (18.7 g, 123 mmol) over one hour at −60° C. After stirring for 30 minutes at −60° C., the reaction system was stirred for 1.5 hour at room temperature. A mixed solution of 200 mL of 2 M sulfuric acid aqueous solution and 100 mL of tetrahydrofuran was added dropwise over one hour to the reaction solution that had been cooled to −50° C. After dropwise addition had been completed, the solution was stirred for 18 hours at room temperature, and 200 mL of ethyl acetate was added. The separated organic layer was washed with (saturated) brine, dried by sodium sulfate, and a crude product was obtained by distilling off the solvent.

Step 4

The crude products obtained in step 2 and step 3 together were subjected to silica gel column chromatography (ethyl acetate/petroleum ether), and 2-chloro-6-methylnicotinaldehyde (42.0 g, 78%) was obtained.

¹H NMR (400 MHz, CDCl₃) δ 2.62 (s, 3H), 7.24 (d, J=8.0 Hz, 1H), 8.1 (d, J=8.0 Hz, 1H), 10.39 (s, 1H).

Step 5

Hydrazine monohydrate (85% aqueous solution, 167 g, 2840 mmol) was added to 600 mL of an aqueous solution of 2-chloro-6-methylnicotinaldehyde (22.0 g, 142 mmol), and heated and refluxed for four days. The approximately 300 mL of suspension obtained by distilling off the solvent under reduced pressure was filtered, and the solid obtained was washed with water to obtain the target 6-methyl-1H-pyrazolo[3,4-b]pyridine (9.20 g). In addition, the filtrate was extracted three times by methylene chloride/methanol (v/v=10/1, 100 mL), and the organic layer was washed with (saturated) brine, and dried with anhydrous sodium sulfate. The residue obtained by distilling off the solvent was subjected to silica gel column chromatography (ethyl acetate/petroleum ether), and 6-methyl-1H-pyrazolo[3,4-b]pyridine (4.90 g) was obtained. In total, the target 6-methyl-1H-pyrazolo[3,4-b]pyridine (14.1 g, 75%) was obtained.

¹H NMR (400 MHz, CDCl₃) δ 2.80 (s, 3H), 7.08 (d, J=8.0 Hz, 1H), 8.04 (d, J=8.0 Hz, 1H), 8.07 (s, 1H), 12.55 (brs, 1H).

Step 6

Iodine (38.0 g, 150 mmol) and potassium hydroxide (17.0 g, 303 mmol) were added over 30 minutes at 0° C. to 200 mL of a dimethylformamide solution of 6-methyl-1H-pyrazolo[3,4-b]pyridine (10.0 g, 38.0 mmol). After being stirred for one hour at 0° C. and for two hours at room temperature, the reaction solution was cooled to 0° C., and 100 mL of saturated sodium pyrosulfite aqueous solution was added. Two hundred milliliters of ethyl acetate and 300 mL of water were also added, and the suspension obtained was filtered. The solid was washed successively with water and acetonitrile, and 3-iodo-6-methyl-1H-pyrazolo[3,4-b]pyridine (9.50 g) was obtained. In addition, the filtrate was extracted three times with ethyl acetate, and the organic layer was washed three times with water, then dried with anhydrous sodium sulfate. The crude product obtained by distilling off the solvent was recrystallized from ethyl acetate, and 3-iodo-6-methyl-1H-pyrazolo[3,4-b]pyridine (6.90 g) was obtained. In total, the target 3-iodo-6-methyl-1H-pyrazolo[3,4-b]pyridine (16.4 g, 84%) was obtained.

¹H NMR (400 MHz, CDCl₃) δ 2.79 (s, 3H), 7.10 (d, J=8.4 Hz, 1H), 7.76 (d, J=8.4 Hz, 1H), 12.86 (brs, 1H).

Step 7

Two hundred milliliters of a tetrahydrofuran solution of 3-iodo-6-methyl-1H-pyrazolo[3,4-b]pyridine (16.0 g, 61.8 mmol) was cooled to 0° C., and isopropyl magnesium chloride (2.0 M tetrahydrofuran solution, 70.0 mL, 140 mmol) was added dropwise over 30 minutes. After dropwise addition had been completed, stirring was continued for one hour at 0° C., and anhydrous dimethylformamide (18.0 g, 246 mmol) was added dropwise over 10 minutes. The reaction solution was stirred for 18 hours at room temperature, and the reaction was stopped by adding water. 2 M hydrochloric acid was added until the pH became 7, and the reaction solution was extracted three times with ethyl acetate/methanol (v/v=10/1) mixed solvent. The organic layer was washed with (saturated) brine, and dried with anhydrous sodium sulfate. The crude product obtained was dissolved in 100 mL of ethyl acetate/methanol (v/v=3/1) mixed solvent and filtered to obtain the target 6-methyl-1H-pyrazolo[3,4-b]pyridine-3-carbaldehyde (1.90 g). The filtrate was reused in step 8.

Step 8

Two hundred twenty milliliters of a tetrahydrofuran solution of 3-iodo-6-methyl-1H-pyrazolo[3,4-b]pyridine (16.0 g, 61.8 mmol) was cooled to 0° C., and isopropyl magnesium chloride (2.0 M tetrahydrofuran solution, 70.0 mL, 140 mmol) was added dropwise over 30 minutes. Following the dropwise addition, stirring was continued for one hour at 0° C., and anhydrous dimethylformamide (18.0 g, 247 mmol) was added dropwise over 10 minutes. The reaction solution was stirred for 18 hours at room temperature, and the reaction was stopped by adding water. 2 M hydrochloric acid was added until the pH reached 7, and the reaction solution was extracted three times with ethyl acetate/methanol (v/v=10/1) mixed solvent. The organic layer was washed with (saturated) brine, and dried with anhydrous sodium sulfate. The solvent was distilled off after adding the filtrate of step 7, and the crude product obtained was filtered after washing with 200 mL of ethyl acetate/tetrahydrofuran (v/v=1/1) mixed solvent, yielding the target 6-methyl-1H-pyrazolo[3,4-b]pyridine-3-carbaldehyde (3.40 g). In total with step 7, the target 6-methyl-1H-pyrazolo[3,4-b]pyridine-3-carbaldehyde (5.30 g, 27%) was obtained.

¹H NMR (400 MHz, DMSO-d₆) δ 2.64 (s, 3H), 7.32 (d, J=8.0 Hz, 1H), 8.39 (d, J=8.0 Hz, 1H), 10.12 (s, 1H), 14.51 (brs, 1H).

Step 9

The 6-methyl-1H-pyrazolo[3,4-b]pyridine-3-carbaldehyde (0.0948 g, 0.588 mmol) obtained in step 8 and piperidine (0.0400 g, 0.470 mmol) were added to 2.5 mL of a methanol solution of the tert-butyl 4-[(6-methoxy-3-oxo-2,3-dihydrobenzofuran-7-yl)methyl]piperazine-1-carboxylate (0.213 g, 0.588 mmol) described in [WO2011/136319], and stirred for two hours at 60° C. After cooling to room temperature, the precipitated solid was filtered out, and the target tert-butyl (Z)-4-({6-methoxy[2-[(6-methyl-1H-pyrazolo[3,4-b]pyridin-3-yl)methylene]-3-oxo-2,3-dihydrobenzofuran-7-yl}methyl)piperazine-1-carboxylate (0.183 g, 62%) was obtained.

¹H NMR (300 MHz, DMSO-d₆) δ 1.37 (s, 9H), 2.44 (t, J=4.5 Hz, 4H), 2.63 (s, 3H), 3.28-3.31 (m, 4H), 3.71 (s, 2H), 3.97 (s, 3H), 6.97 (s, 1H), 7.06 (d, J=9.0 Hz, 1H), 7.19 (d, J=7.8 Hz, 1H), 7.78 (d, J=8.7 Hz, 1H), 8.83 (d, J=8.1 Hz, 1H), 14.15 (brs, 1H).

Step 10

Four milliliters of trifluoroacetic acid was added at room temperature to 4 mL of a methylene chloride solution of tert-butyl (Z)-4-({6-methoxy[2-[(6-methyl-1H-pyrazolo[3,4-b]pyridin-3-yl)methylene]-3-oxo-2,3-dihydrobenzofuran-7-yl}methyl)piperazine-1-carboxylate (0.177 g, 0.350 mmol). After stirring for 16 hours at room temperature, the solvent was distilled off under reduced pressure. Saturated sodium hydrogen carbonate aqueous solution was added to the residue obtained until the residue became basic, and the precipitated solid was filtered out. The solid was washed by water and dried under reduced pressure to obtain the target (Z)-6-methoxy-2-[(6-methyl-1H-pyrazolo[3,4-b]pyridin-3-yl)methylene]-7-(piperazin-1-ylmethyl)benzofuran-3(2H)-one (0.0694 g, 49%).

¹H NMR (300 MHz, DMSO-d₆) δ 2.59-2.65 (m, 7H), 2.97-3.01 (m, 4H), 3.76 (s, 2H), 3.98 (s, 3H), 7.00 (s, 1H), 7.08 (d, J=8.7 Hz, 1H), 7.22 (d, J=9.0 Hz, 1H), 7.81 (d, J=8.7 Hz, 1H), 8.82 (d, J=8.1 Hz, 1H).

### Synthesis Example 3

### Synthesis of compound 8 ((R,Z)-2-[(1H-pyrazolo[3,4-b]pyridin-3-yl)methylene]-6-methoxy-7-[(2-methylpiperazin-1-yl)methyl]benzofuran-3(2H)-one)

Compound 8 was synthesized by synthesis scheme 3 below.

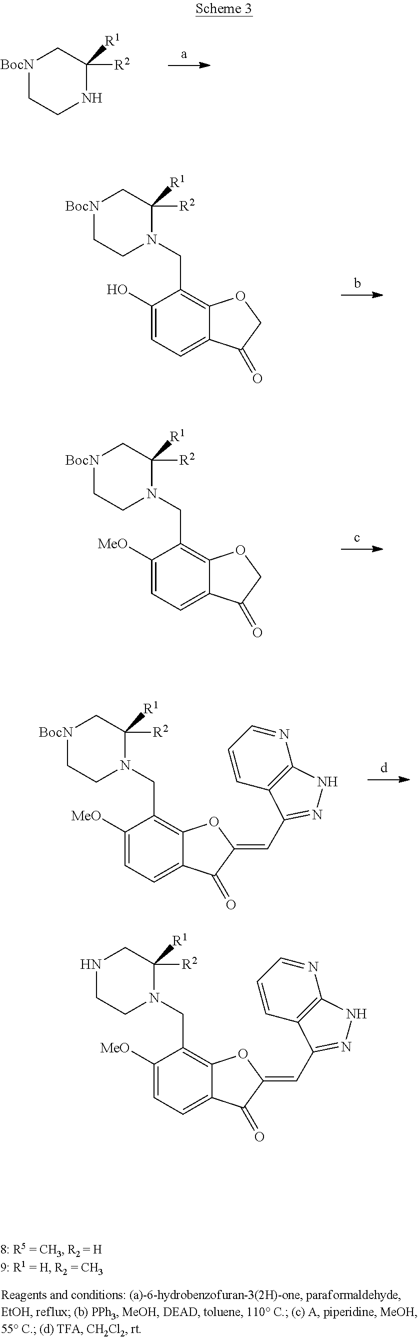

(a) Step 1

Paraformaldehyde (0.300 g, 10.0 mmol) and 6-hydroxybenzofuran-3(2H)-one (1.50 g, 10.0 mmol) were added to 10 mL of an ethanol solution of tert-butyl (R)-3-methylpiperazine-1-carboxylate (2.00 g, 10.0 mmol), and heated and refluxed for seven hours. The residue obtained by concentrating the reaction solution was subjected to silica gel chromatography (ethyl acetate/hexane), and 2.00 g of a crude product was obtained. The crude product was washed by methylene chloride, and the target tert-butyl (R)-4-[(6-hydroxy-3-oxo-2,3-dihydrobenzofuran-7-yl)methyl]-3-methylpiperazine-1-carboxylate (1.58 g, 44%) was obtained.

¹H NMR (300 MHz, DMSO-d₆) δ 1.10 (d, J=6.0 Hz, 3H), 1.39 (s, 9H), 2.22-2.30 (m, 1H), 2.54-2.61 (m, 1H), 2.70-2.77 (m, 1H), 2.87-3.02 (m, 1H), 3.09-3.17 (m, 1H), 3.44-3.51 (m, 2H), 3.56 (d, J=13.9 Hz, 1H), 4.01 (d, J=13.9 Hz, 1H), 4.73 (s, 2H), 6.56 (d, J=8.8 Hz, 1H), 7.39 (d, J=8.8 Hz, 1H).

(b) Step 2

Triphenyl phosphine (1.68 g, 6.42 mmol), methanol (0.208 mL, 5.14 mmol), and diethyl azodicarboxylate (40% toluene solution, 2.80 g, 6.42 mmol) were added to 17 mL of a toluene solution of tert-butyl (R)-4-[(6-hydroxy-3-oxo-2,3-dihydrobenzofuran-7-yl)methyl]-3-methylpiperazine-1-carboxylate (1.55 g, 4.28 mmol), and stirred for five hours at 110° C. The residue obtained by concentrating the reaction solution was subjected to silica gel chromatography (ethyl acetate/chloroform), and the crude product obtained was further purified by silica gel chromatography (ethyl acetate/hexane) to obtain tert-butyl (R)-4-[(6-methoxy-3-oxo-2,3-dihydrobenzofuran-7-yl)methyl]-3-methylpiperazine-1-carboxylate (0.900 g, 56%).

¹H NMR (300 MHz, CDCl₃) δ 1.23 (d, J=6.0 Hz, 3H), 1.45 (s, 9H), 2.17-2.24 (m, 1H), 2.37-2.48 (m, 1H), 2.73-2.80 (m, 2H), 2.97-3.06 (m, 1H), 3.41 (d, J=12.6 Hz, 1H), 3.68-3.73 (m, 2H), 3.93 (s, 3H), 4.00 (d, J=12.6 Hz, 1H), 4.64 (s, 2H), 6.70 (d, J=8.7 Hz, 1H), 7.62 (d, J=8.7 Hz, 1H).

(c) Step 3

The 1H-pyrazolo[3,4-b]pyridine-3-carbaldehyde (0.0918 g, 0.624 mmol) described in [WO2011/136319] was added to 2.5 mL of a methanol solution of tert-butyl (R)-4-[(6-methoxy-3-oxo-2,3-dihydrobenzofuran-7-yl)methyl]-3-methylpiperazine-1-carboxylate (0.226 g, 0.64 mmol). After adding piperidine (0.0425 g, 0.499 mmol), the reaction solution was stirred for two hours at 60° C., and cooled to room temperature. The precipitated solid was filtered out, and tert-butyl (R,Z)-4-({2-[(1-H-pyrazolo[3,4-b]pyridin-3-yl)methylene]-6-methoxy-3-oxo-2,3-dihydrobenzofuran-7-yl}methyl)-3-methylpiperazine-1-carboxylate (0.195 g, 62%) was obtained.

¹H NMR (300 MHz, DMSO-d₆) δ 1.15 (d, J=5.7 Hz, 3H), 1.35 (s, 9H), 2.13-2.19 (m, 1H), 2.52-2.68 (m, 2H), 2.88-3.10 (m, 2H), 3.28 (m, 1H), 3.44-3.48 (m, 1H), 3.52 (d, J=12.3 Hz, 1H), 3.97 (s, 3H), 4.04 (d, J=12.3 Hz, 1H), 7.04-7.07 (m, 2H), 7.33 (dd, J=4.5, 8.1 Hz, 1H), 7.79 (d, J=8.1 Hz, 1H), 8.63 (dd, J=1.5, 4.5 Hz, 1H), 8.94 (dd, J=1.5, 8.1 Hz, 1H), 14.36 (brs, 1H).

(d) Step 4

Two milliliters of trifluoroacetic acid was added to 2 mL of a methylene chloride solution of tert-butyl (R,Z)-4-({2-[(1-H-pyrazolo[3,4-b]pyridin-3-yl)methylene]-6-methoxy-3-oxo-2,3-dihydrobenzofuran-7-yl}methyl)-3-methylpiperazine-1-carboxylate (0.190 g, 0.376 mmol), and stirred for 18 hours at room temperature. Eight milliliters of saturated sodium hydrogen carbonate was added to the residue obtained by concentrating the reaction solution, and the precipitated solid was filtered out. After washing with water, the solid was dried under reduced pressure to obtain the target (R,Z)-2-[(1H-pyrazolo[3,4-b]pyridin-3-yl)methylene]-6-methoxy-7-[(2-methylpiperazin-1-yl)methyl]benzofuran-3(2H)-one (0.0909 g, 60%).

¹H NMR (300 MHz, DMSO-d₆) δ 1.22 (d, J=5.1 Hz, 3H), 2.22-2.30 (m, 1H), 2.54-2.61 (m, 2H), 2.65-2.76 (m, 2H), 2.84-2.88 (m, 1H), 2.94-3.03 (m, 1H), 3.48 (d, J=12.6 Hz, 1H), 3.98 (s, 3H), 4.12 (d, J=12.6 Hz, 1H), 7.06 (s, 1H), 7.07 (d, J=8.7 Hz, 1H), 7.37 (dd, J=4.5, 8.1 Hz, 1H), 7.81 (d, J=8.7 Hz, 1H), 8.65 (dd, J=1.5, 4.5 Hz, 1H), 8.92 (dd, J=1.5, 8.1 Hz, 1H).

### Synthesis Example 4

### Synthesis of compound 9 ((S,Z)-2-[(1H-pyrazolo[3,4-b]pyridin-3-yl)methylene]-6-methoxy-7-[(2-methylpiperazin-1-yl)methyl]benzofuran-3(2H)-one)

Compound 9 was synthesized by scheme 3 above.

(a) Step 1

Paraformaldehyde (0.300 g, 10.0 mmol) and 6-hydroxybenzofuran-3(2H)-one (1.50 g, 10.0 mmol) were added to 10 mL of an ethanol solution of tert-butyl (S)-3-methylpiperazine-1-carboxylate (2.00 g, 10.0 mmol), and heated and refluxed for seven hours. The residue obtained by concentrating the reaction solution was subjected to silica gel chromatography (ethyl acetate/hexane), and 2.20 g of a crude product was obtained. The crude product was washed with chloroform, and the target tert-butyl (S)-4-[(6-hydroxy-3-oxo-2,3-dihydrobenzofuran-7-yl)methyl]-3-methylpiperazine-1-carboxylate (1.78 g, 49%) was obtained.

¹H NMR (300 MHz, DMSO-d₆) δ 1.10 (d, J=6.0 Hz, 3H), 1.39 (s, 9H), 2.22-2.30 (m, 1H), 2.54-2.61 (m, 1H), 2.70-2.77 (m, 1H), 2.87-3.02 (m, 1H), 3.09-3.17 (m, 1H), 3.44-3.51 (m, 2H), 3.56 (d, J=13.9 Hz, 1H), 4.01 (d, J=13.9 Hz, 1H), 4.73 (s, 2H), 6.56 (d, J=8.8 Hz, 1H), 7.39 (d, J=8.8 Hz, 1H).

(b) Step 2

Triphenyl phosphine (1.87 g, 7.13 mmol), methanol (0.231 mL, 5.70 mmol), and diethyl azodicarboxylate (40% toluene solution, 3.10 g, 7.13 mmol) were added to 19 mL of a toluene solution of tert-butyl (S)-4-[(6-hydroxy-3-oxo-2,3-dihydrobenzofuran-7-yl)methyl]-3-methylpiperazine-1-carboxylate (1.72 g, 4.75 mmol), and stirred for five hours at 110° C. The residue obtained by concentrating the reaction solution was subjected to silica gel chromatography (ethyl acetate/chloroform). The crude product obtained was further purified by silica gel chromatography (ethyl acetate/hexane), and tert-butyl (S)-4-[(6-methoxy-3-oxo-2,3-dihydrobenzofuran-7-yl)methyl]-3-methylpiperazine-1-carboxylate (0.968 g, 56%) was obtained.

¹H NMR (300 MHz, CDCl₃) δ 1.23 (d, J=6.0 Hz, 3H), 1.45 (s, 9H), 2.17-2.24 (m, 1H), 2.37-2.48 (m, 1H), 2.73-2.80 (m, 2H), 2.97-3.06 (m, 1H), 3.41 (d, J=12.6 Hz, 1H), 3.68-3.73 (m, 2H), 3.93 (s, 3H), 4.00 (d, J=12.6 Hz, 1H), 4.64 (s, 2H), 6.70 (d, J=8.7 Hz, 1H), 7.62 (d, J=8.7 Hz, 1H).

(c) Step 3

The 1H-pyrazolo[3,4-b]pyridine-3-carbaldehyde (0.0845 g, 0.574 mmol) described in [WO2011/136319] was added to 2.3 mL of a methanol solution of tert-butyl (S)-4-[(6-methoxy-3-oxo-2,3-dihydrobenzofuran-7-yl)methyl]-3-methylpiperazine-1-carboxylate (0.208 g, 0.574 mmol). After adding piperidine (0.0391 g, 0.459 mmol), the reaction solution was stirred for two hours at 60° C. and cooled to room temperature. The precipitated solid was filtered out, and tert-butyl (S,Z)-4-({2-[(1H-pyrazolo[3,4-b]pyridin-3-yl)methylene]-6-methoxy-3-oxo-2,3-dihydrobenzofuran-7-yl}methyl)-3-methylpiperazine-1-carboxylate (0.197 g, 68%) was obtained.

¹H NMR (300 MHz, DMSO-d₆) δ 1.15 (d, J=5.7 Hz, 3H), 1.35 (s, 9H), 2.13-2.19 (m, 1H), 2.52-2.68 (m, 2H), 2.88-3.10 (m, 2H), 3.28 (m, 1H), 3.44-3.48 (m, 1H), 3.52 (d, J=12.3 Hz, 1H), 3.97 (s, 3H), 4.04 (d, J=12.3 Hz, 1H), 7.04-7.07 (m, 2H), 7.33 (dd, J=4.5, 8.1 Hz, 1H), 7.79 (d, J=8.1 Hz, 1H), 8.63 (dd, J=1.5, 4.5 Hz, 1H), 8.94 (dd, J=1.5, 8.1 Hz, 1H), 14.36 (brs, 1H).

(d) Step 4

Two milliliters of trifluoroacetic acid was added to 2 mL of a methylene chloride solution of tert-butyl (S,Z)-4-({2-[(1H-pyrazolo[3,4-b]pyridin-3-yl)methylene]-6-methoxy-3-oxo-2,3-dihydrobenzofuran-7-yl}methyl)-3-methylpiperazine-1-carboxylate (0.183 g, 0.362 mmol), and stirred for 18 hours at room temperature. Ten milliliters of saturated sodium hydrogen carbonate was added to the residue obtained by concentrating the reaction solution, and the precipitated solid was filtered out. After washing with water, the solid was dried under reduced pressure to obtain the target (S,Z)-2-[(1H-pyrazolo[3,4-b]pyridin-3-yl)methylene]-6-methoxy-7-[(2-methylpiperazin-1-yl)methyl]benzofuran-3(2H)-one (0.103 g, 70%).

¹H NMR (300 MHz, DMSO-d₆) δ 1.22 (d, J=5.1 Hz, 3H), 2.22-2.30 (m, 1H), 2.54-2.61 (m, 2H), 2.65-2.76 (m, 2H), 2.84-2.88 (m, 1H), 2.94-3.03 (m, 1H), 3.48 (d, J=12.6 Hz, 1H), 3.98 (s, 3H), 4.12 (d, J=12.6 Hz, 1H), 7.06 (s, 1H), 7.07 (d, J=8.7 Hz, 1H), 7.37 (dd, J=4.5, 8.1 Hz, 1H), 7.81 (d, J=8.7 Hz, 1H), 8.65 (dd, J=1.5, 4.5 Hz, 1H), 8.92 (dd, J=1.5, 8.1 Hz, 1H).

### Synthesis Example 5

### Synthesis of compound 10 ((Z)-2-[(1H-pyrazolo[3,4-b]pyridin-3-yl)methylene]-7-[(cis-3,5-dimethylpiperazin-1-yl)methyl]-6-methoxybenzofuran-3(2H)-one)

Compound 10 was synthesized by synthesis scheme 4 below.

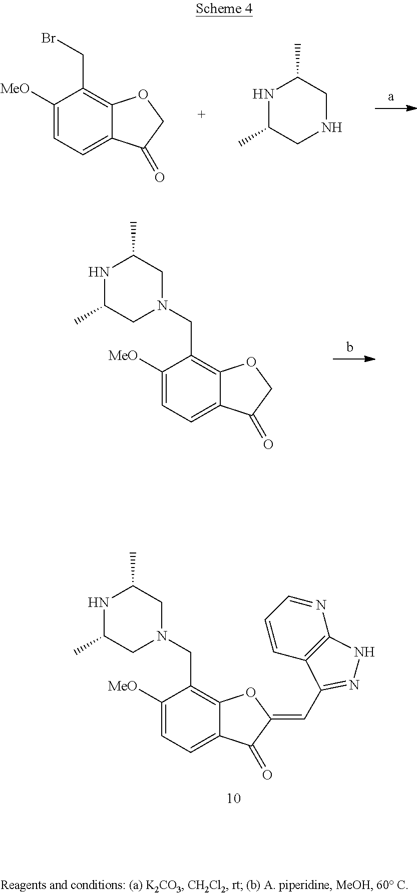

(a) Step 1

cis-2,6-Dimethylpiperazine (0.114 g, 1.00 mmol) and potassium carbonate (0.0691 g, 0.500 mmol) were added to 4 mL of methylene chloride, and stirred at room temperature. Four milliliters of a methylene chloride solution of the 7-(bromomethyl)-6-methoxybenzofuran-3(2H)-one (0.129 g, 0.500 mmol) described in [WO2011/136319] was added dropwise, and stirring was continued for 12 hours at room temperature. The reaction solution was filtered, and the residue obtained by concentrating the filtrate was purified by silica gel chromatography (methanol/chloroform), and 7-[(cis-3,5-dimethylpiperazin-1-yl)methyl]-6-methoxybenzofuran-3(2H)-one (0.131 g, 90%) was obtained.

¹H NMR (300 MHz, CDCl₃) δ 1.03 (d, J=6.6 Hz, 6H), 1.73 (t, J=11.1 Hz, 2H), 2.82 (dd, J=2.1, 11.1 Hz, 2H), 2.87-2.98 (m, 2H), 3.67 (s, 2H), 3.93 (s, 3H), 4.64 (s, 2H), 6.70 (d, J=9.0 Hz, 1H), 7.62 (d, J=9.0 Hz, 1H).

(b) Step 2

The 1H-pyrazolo[3,4-b]pyridine-3-carbaldehyde (0.0388 g, 0.264 mmol) described in [WO2011/136319] was added to 1 mL of a methanol solution of 7-[(cis-3,5-dimethylpiperazin-1-yl)methyl]-6-methoxybenzofuran-3(2H)-one (0.0767 g, 0.264 mmol). After adding piperidine (0.0180 g, 0.211 mmol), the reaction solution was stirred for two hours at 60° C., and cooled to room temperature. The precipitated solid was filtered out, and the target (Z)-2-[(1H-pyrazolo[3,4-b]pyridin-3-yl)methylene]-7-[(cis-3,5-dimethylpiperazin-1-yl)methyl]-6-methoxybenzofuran-3(2H)-one (0.0673 g, 61%) was obtained.

¹H NMR (300 MHz, CDCl₃) δ 0.89 (d, J=6.6 Hz, 6H), 1.64 (t, J=10.2 Hz, 2H), 2.64-2.76 (m, 4H), 3.66 (s, 2H), 3.97 (s, 3H), 7.01 (s, 1H), 7.06 (d, J=8.7 Hz, 1H), 7.25 (dd, J=4.5, 8.1 Hz, 1H), 7.79 (d, J=8.7 Hz, 1H), 8.65 (dd, J=1.5, 4.5 Hz, 1H), 9.02 (dd, J=1.5, 8.1 Hz, 1H).

### Synthesis Example 6

### Synthesis of compound 11 ((S,Z)-2-[(1H-pyrazolo[3,4-b]pyridin-3-yl)methylene]-6-methoxy-7-[(3-methylpiperazin-1-yl)methyl]benzofuran-3(2H)-one)

Compound 11 was synthesized by synthesis scheme 5 below.

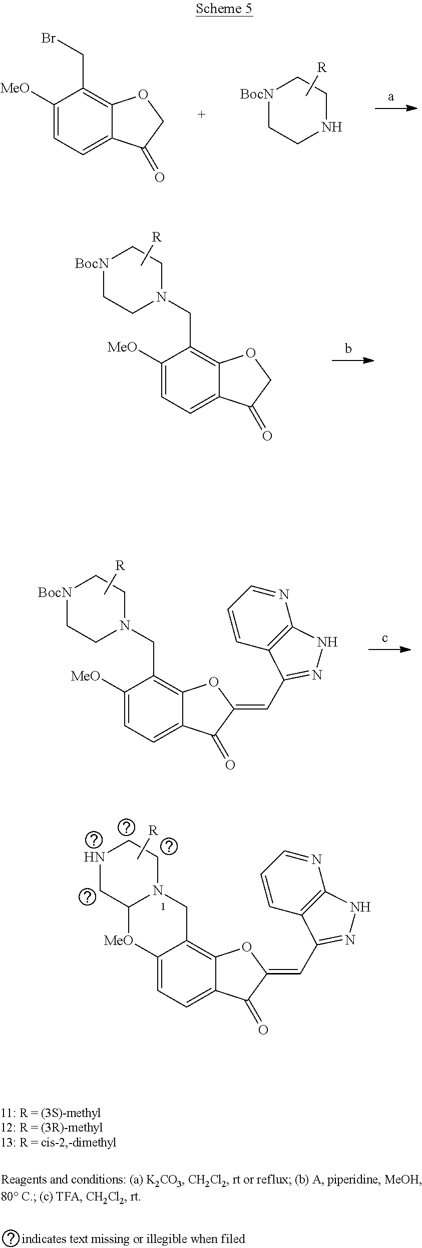

(a) Step 1

The 7-(bromomethyl)-6-methoxybenzofuran-3(2H)-one (0.514 g, 2.00 mmol) described in [WO2011/136319] and potassium carbonate (0.276 g, 2.00 mmol) were added to 8 mL of methylene chloride and stirred at room temperature. Two milliliters of a methylene chloride solution of tert-butyl (S)-2-methylpiperazine-1-carboxylate (0.401 g, 2.00 mmol) was added dropwise, and stirring was continued for 24 hours at room temperature. The reaction solution was filtered, and the residue obtained by concentrating the filtrate was purified by silica gel chromatography (ethyl acetate/hexane) to obtain tert-butyl (S)-4-[(6-methoxy-3-oxo-2,3-dihydrobenzofuran-7-yl)methyl]-2-methylpiperazine-1-carboxylate (0.531 g, 70%).

¹H NMR (300 MHz, CDCl₃) δ 1.12 (d, J=6.6 Hz, 3H), 1.36 (s, 9H), 1.98 (dt, J=0.6, 11.7 Hz, 1H), 2.17 (dd, J=4.2, 10.8 Hz, 1H), 2.57 (d, J=10.8 Hz, 1H), 2.72 (d, J=10.8 Hz, 1H), 2.97 (dt, J=3.9, 12.3 Hz, 1H), 3.60 (d, J=2.4 Hz, 2H), 3.69 (d, J=12.3 Hz, 1H), 3.85 (s, 3H), 4.09-4.14 (m, 1H), 4.55 (s, 2H), 6.62 (d, J=8.7 Hz, 1H), 7.54 (d, J=8.7 Hz, 1H).

(b) Step 2

The 1H-pyrazolo-[3,4-b]pyridine-3-carbaldehyde (0.0368 g, 0.250 mmol) described in [WO2011/136319] was added to 1 mL of a methanol solution of tert-butyl (S)-4-[(6-methoxy-3-oxo-2,3-dihydrobenzofuran-7-yl)methyl]-2-methylpiperazine-1-carboxylate (0.0941 g, 0.250 mmol). After adding piperidine (0.0170 g, 0.200 mmol), the reaction solution was stirred for two hours at 60° C. and cooled to room temperature. The precipitated solid was filtered out, and tert-butyl (S,Z)-4-({2-[(1H-pyrazolo[3,4-b]pyridin-3-yl)methylene]-6-methoxy-3-oxo-2,3-dihydrobenzofuran-7-yl}methyl)-2-methylpiperazine-1-carboxylate (0.0442 g, 35%) was obtained.

¹H NMR (300 MHz, DMSO-d₆) δ 0.92 (d, J=6.6 Hz, 3H), 1.35 (s, 9H), 1.97-2.06 (m, 1H), 2.15 (dd, J=3.6, 11.1 Hz, 1H), 2.66 (d, J=11.7 Hz, 1H), 2.84-2.92 (m, 2H), 3.65-3.70 (m, 1H), 3.75 (s, 2H), 3.98 (s, 3H), 4.02-4.08 (m, 1H), 7.03 (s, 1H), 7.07 (d, J=8.1 Hz, 1H), 7.40 (dd, J=4.5, 8.1 Hz, 1H), 7.81 (d, J=8.7 Hz, 1H), 8.64 (dd, J=1.5, 4.5 Hz, 1H), 9.02 (dd, J=1.5, 8.1 Hz, 1H), 14.07 (brs, 1H).

(c) Step 3

Two milliliters of trifluoroacetic acid was added to 2 mL of a methylene chloride solution of tert-butyl (S,Z)-4-({2-[(1H-pyrazolo[3,4-b]pyridin-3-yl)methylene]-6-methoxy-3-oxo-2,3-dihydrobenzofuran-7-yl}methyl)-2-methylpiperazine-1-carboxylate (0.0424 g, 0.0839 mmol), and stirred for 18 hours at room temperature. Four milliliters of saturated sodium hydrogen carbonate was added to the residue obtained by concentrating the reaction solution, and the precipitated solid was filtered out. After washing with water, the solid was dried under reduced pressure to obtain the target (S,Z)-2-[(1H-pyrazolo[3,4-b]pyridin-3-yl)methylene]-6-methoxy-7-[(3-methylpiperazine-1-yl)methyl]benzofuran-3(2H)-one (0.0154 g, 45%).

¹H NMR (300 MHz, DMSO-d₆) δ 0.96 (d, J=6.6 Hz, 3H), 1.85 (t, J=10.8 Hz, 1H), 2.12 (dt, J=2.1, 10.8 Hz, 1H), 2.66-2.93 (m, 5H), 3.72 (s, 2H), 3.98 (s, 3H), 7.03 (s, 1H), 7.08 (d, J=9.0 Hz, 1H), 7.31 (dd, J=4.5, 8.1 Hz, 1H), 7.81 (d, J=8.7 Hz, 1H), 8.65 (dd, J=1.5, 4.5 Hz, 1H), 9.01 (dd, J=1.5, 8.7 Hz, 1H).

### Synthesis Example 7

### Synthesis of compound 12 ((R,Z)-2-[(1H-pyrazolo[3,4-b]pyridin-3-yl)methylene]-6-methoxy-7-[(3-methylpiperazin-1-yl)methyl]benzofuran-3(2H)-one)

Compound 12 was synthesized by synthesis scheme 5 above.

(a) Step 1

The 7-(bromomethyl)-6-methoxybenzofuran-3(2H)-one (0.514 g, 2.00 mmol) described in [WO2011/136319] and potassium carbonate (0.276 g, 2.00 mmol) were added to 8 mL of methylene chloride, and stirred at room temperature. Two milliliters of a methylene chloride solution of tert-butyl (R)-2-methylpiperazine-1-carboxylate (0.401 g, 2.00 mmol) was added dropwise, and stirring was continued for 24 hours at room temperature. The reaction solution was filtered, and the residue obtained by concentrating the filtrate was purified by silica gel chromatography (ethyl acetate/hexane) to obtain tert-butyl (R)-4-[(6-methoxy-3-oxo-2,3-dihydrobenzofuran-7-yl)methyl]-2-methylpiperazine-1-carboxylate (0.314 g, 42%).

¹H NMR (300 MHz, CDCl₃) δ 1.12 (d, J=6.6 Hz, 3H), 1.36 (s, 9H), 1.98 (dt, J=0.6, 11.7 Hz, 1H), 2.17 (dd, J=4.2, 10.8 Hz, 1H), 2.57 (d, J=10.8 Hz, 1H), 2.72 (d, J=10.8 Hz, 1H), 2.97 (dt, J=3.9, 12.3 Hz, 1H), 3.60 (d, J=2.4 Hz, 2H), 3.69 (d, J=12.3 Hz, 1H), 3.85 (s, 3H), 4.09-4.14 (m, 1H), 4.55 (s, 2H), 6.62 (d, J=8.7 Hz, 1H), 7.54 (d, J=8.7 Hz, 1H).

(b) Step 2

The 1H-pyrazolo[3,4-b]pyridine-3-carbaldehyde (0.0597 g, 0.406 mmol) described in [WO2011/136319] was added to 1.6 mL of a methanol solution of tert-butyl (R)-4-[(6-methoxy-3-oxo-2,3-dihydrobenzofuran-7-yl)methyl]-2-methylpiperazine-1-carboxylate (0.153 g, 0.406 mmol). After adding piperidine (0.0277 g, 0.325 mol), the reaction solution was stirred for two hours at 60° C. and concentrated. The residue obtained was purified by silica gel chromatography (methanol/chloroform) to obtain tert-butyl (R,Z)-4-({2-[(1H-pyrazolo[3,4-b]pyridin-3-yl)methylene]-6-methoxy-3-oxo-2,3-dihydrobenzofuran-7-yl}methyl)-2-methylpiperazine-1-carboxylate (0.119 g, 58%).

¹H NMR (300 MHz, DMSO-d₆) δ 0.92 (d, J=6.6 Hz, 3H), 1.35 (s, 9H), 1.97-2.06 (m, 1H), 2.15 (dd, J=3.6, 11.1 Hz, 1H), 2.66 (d, J=11.7 Hz, 1H), 2.84-2.92 (m, 2H), 3.65-3.70 (m, 1H), 3.75 (s, 2H), 3.98 (s, 3H), 4.02-4.08 (m, 1H), 7.03 (s, 1H), 7.07 (d, J=8.1 Hz, 1H), 7.40 (dd, J=4.5, 8.1 Hz, 1H), 7.81 (d, J=8.7 Hz, 1H), 8.64 (dd, J=1.5, 4.5 Hz, 1H), 9.02 (dd, J=1.5, 8.1 Hz, 1H), 14.07 (brs, 1H).

(c) Step 3

Four milliliters of trifluoroacetic acid was added to 4 mL of a methylene chloride solution of tert-butyl (R,Z)-4-({2-[(1H-pyrazolo[3,4-b]pyridin-3-yl)methylene]-6-methoxy-3-oxo-2,3-dihydrobenzofuran-7-yl}methyl)-2-methylpiperazine-1-carboxylate (0.108 g, 0.214 mmol), and stirred for 16 hours at room temperature. Six milliliters of saturated sodium hydrogen carbonate was added to the residue obtained by concentrating the reaction solution, and the precipitated solid was filtered out. After washing with water, the solid was dried under reduced pressure to obtain the target (R,Z)-2-[(1H-pyrazolo[3,4-b]pyridin-3-yl)methylene]-6-methoxy-7-[(3-methylpiperazin-1-yl)methyl]benzofuran-3(2H)-one (0.0533 g, 61%).

¹H NMR (300 MHz, DMSO-d₆) δ 0.96 (d, J=6.6 Hz, 3H), 1.85 (t, J=10.8 Hz, 1H), 2.12 (dt, J=2.1, 10.8 Hz, 1H), 2.66-2.93 (m, 5H), 3.72 (s, 2H), 3.98 (s, 3H), 7.03 (s, 1H), 7.08 (d, J=9.0 Hz, 1H), 7.31 (dd, J=4.5, 8.1 Hz, 1H), 7.81 (d, J=8.7 Hz, 1H), 8.65 (dd, J=1.5, 4.5 Hz, 1H), 9.01 (dd, J=1.5, 8.7 Hz, 1H).

### Synthesis Example 8

### Synthesis of compound 13 ((Z)-2-[(1H-pyrazolo[3,4-b]pyridin-3-yl)methylene]-7-[(cis-2,6-dimethylpiperazin-1-yl)methyl]-6-methoxybenzofuran-3(2H)-one)

Compound 13 was synthesized by scheme 5 above.

(a) Step 1

The 7-(bromomethyl)-6-methoxybenzofuran-3(2H)-one (0.514 g, 2.00 mmol) described in [WO2011/136319], tert-butyl cis-3,5-dimethylpiperazine-1-carboxylate (0.429 g, 2.00 mmol), and potassium carbonate (0.346 g, 2.50 mmol) were added to 8 mL of methylene chloride, and heated and refluxed for 15 hours. The reaction solution was filtered, and the residue obtained by concentrating the filtrate was purified by silica gel chromatography (ethyl acetate/hexane) to obtain tert-butyl cis-4-[(6-methoxy-3-oxo-2,3-dihydrobenzofuran-7-yl)methyl]-3,5-dimethylpiperazine-1-carboxylate (0.459 g, 59%).

¹H NMR (300 MHz, CDCl₃) δ 1.18 (d, J=5.7 Hz, 6H), 1.44 (s, 9H), 2.60-2.70 (m, 2H), 2.83-2.90 (m, 2H), 3.60-3.63 (m, 2H), 3.92 (s, 3H), 3.93 (s, 2H), 4.63 (s, 2H), 6.69 (d, J=9.0 Hz, 1H), 7.60 (d, J=9.0 Hz, 1H).

(b) Step 2

The 1H-pyrazolo[3,4-b]pyridine-carbaldehyde (0.0724 g, 0.492 mmol) described in [WO2011-136319] was added to 2 mL of a methanol solution of tert-butyl cis-4-[(6-methoxy-3-oxo-2,3-dihydrobenzofuran-7-yl)methyl]-3,5-dimethylpiperazine-1-carboxylate (0.192 g, 0.492 mmol). After adding piperidine (0.0335 g, 0.394 mmol), the reaction solution was stirred for two hours at 60° C. and cooled to room temperature. The precipitated solid was filtered out, and tert-butyl cis-4-({(Z)-2-[(1H-pyrazolo[3,4-b]pyridin-3-yl)methylene]-6-methoxy-3-oxo-2,3-dihydrobenzofuran-7-yl}methyl)-3,5-dimethylpiperazine-1-carboxylate (0.165 g, 65%) was obtained.

¹H NMR (300 MHz, DMSO-d₆) δ 1.05 (d, J=5.7 Hz, 6H), 1.36 (s, 9H), 2.73-2.80 (m, 2H), 3.17 (d, J=4.2 Hz, 4H), 3.97 (s, 5H), 7.05 (d, J=8.7 Hz, 1H), 7.08 (s, 1H), 7.35 (dd, J=4.5, 8.1 Hz, 1H), 7.78 (d, J=9.0 Hz, 1H), 8.63 (dd, J=1.5, 4.5 Hz, 1H), 8.85 (d, J=8.1 Hz, 1H), 14.36 (brs, 1H).

Step (3)

Six milliliters of trifluoroacetic acid was added to 6 mL of a methylene chloride solution of tert-butyl cis-4-({(Z)-2-[(1H-pyrazolo[3,4-b]pyridin-3-yl)methylene]-6-methoxy-3-oxo-2,3-dihydrobenzofuran-7-yl}methyl)-3,5-dimethylpiperazine-1-carboxylate (0.164 g, 0.316 mmol), and stirred for 14 hours at room temperature. Eight milliliters of saturated sodium hydrogen carbonate was added to the residue obtained by concentrating the reaction solution, and the precipitated solid was filtered out. After washing with water, the solid was dried under reduced pressure to obtain the target (Z)-2-[(1H-pyrazolo[3,4-b]pyridin-3-yl)methylene]-7-[(cis-2,6-dimethylpiperazin-1-yl)methyl]-6-methoxybenzofuran-3(2H)-one (0.108 g, 81%).

¹H NMR (300 MHz, CD₃OD) δ 1.15 (d, J=5.7 Hz, 6H), 2.44-2.52 (m, 2H), 2.58-2.68 (m, 2H), 2.77-2.82 (m, 2H), 3.99 (s, 3H), 4.12 (s, 2H), 6.98 (d, J=8.7 Hz, 1H), 7.10 (s, 1H), 7.37 (dd, J=4.5, 8.1 Hz, 1H), 7.73 (d, J=8.7 Hz, 1H), 8.58 (dd, J=1.5, 4.5 Hz, 1H), 8.76 (dd, J=1.5, 8.1 Hz, 1H).

### Synthesis Example 9

### Synthesis of compound 14 ((S,Z)-2-[(1H-pyrazolo[3,4-b]pyridin-3-yl)methylene]-7-[(3-aminopyrrolidin-1-yl)methyl]-6-methoxybenzofuran-3(2H)-one)

Compound 14 was synthesized by synthesis scheme 6 below.

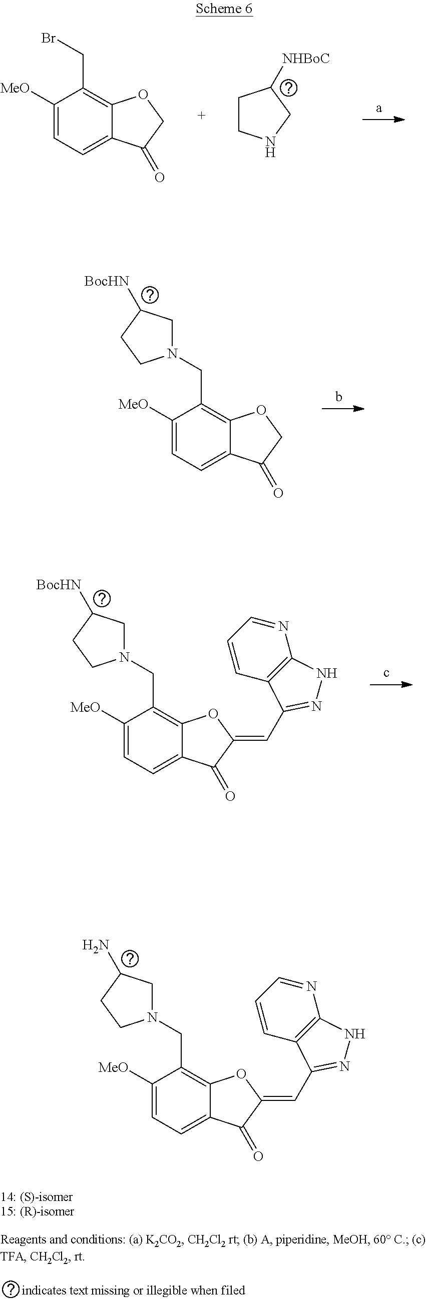

(a) Step 1

The 7-(bromomethyl)-6-methoxybenzofuran-3(2H)-one (0.514 g, 2.00 mmol) described in the known literature [WO2011/136319] and potassium carbonate (0.276 g, 2.00 mmol) were added to 8 mL of methylene chloride, and stirred at room temperature. Four milliliters of a methylene chloride solution of tert-butyl (S)-pyrrolidin-3-ylcarbamate (0.373 g, 2.00 mmol) was added dropwise, and stirring was continued for 15 hours at room temperature. The reaction solution was filtered, and the residue obtained by concentrating the filtrate was purified by silica gel chromatography (methanol/chloroform) to obtain tert-butyl (S)-{1-[(6-methoxy-3-oxo-2,3-dihydrobenzofuran-7-yl)methyl]pyrrolidin-3-yl}carbamate (0.388 g, 54%).

¹H NMR (300 MHz, DMSO-d₆) δ 1.35 (s, 9H), 1.43-1.56 (m, 1H), 1.90-2.02 (m, 1H), 2.22-2.27 (m, 1H), 2.45-2.47 (m, 1H), 2.55 (m, 1H), 2.70-2.75 (m, 1H), 3.60 (s, 2H), 3.79-3.87 (m, 1H), 3.90 (s, 3H), 4.77 (s, 2H), 6.87-6.90 (m, 2H), 7.58 (d, J=8.7 Hz, 1H).

(b) Step 2

The 1H-pyrazolo[3,4-b]pyridine-3-carbaldehyde (0.0727 g, 0.494 mmol) described in [WO2011/136319] was added to 2 mL of a methanol solution of tert-butyl (S)-{1-[(6-methoxy-3-oxo-2,3-dihydrobenzofuran-7-yl)methyl]pyrrolidin-3-yl}carbamate (0.179 g, 0.494 mmol). After adding piperidine (0.0336 g, 0.395 mmol), the reaction system was stirred for two hours at 60° C. The residue obtained by concentrating the reaction solution was purified by silica gel chromatography (methanol/chloroform) to obtain tert-butyl (S,Z)-[1-({2-[(1H-pyrazolo[3,4-b]pyridin-3-yl)methylene]-6-methoxy-3-oxo-2,3-dihydrobenzofuran-7-yl}methyl)pyrrolidin-3-yl]carbamate (0.150 g, 62%).

¹H NMR (300 MHz, DMSO-d₆) δ 1.31-1.39 (m, 1H), 1.36 (s, 9H), 1.51-1.61 (m, 1H), 1.95-2.07 (m, 1H), 2.34-2.39 (m, 1H), 2.58-2.62 (m, 1H), 2.83-2.88 (m, 1H), 3.81 (s, 2H), 3.85-3.91 (m, 1H), 3.98 (s, 3H), 6.92 (d, J=6.6 Hz, 1H), 7.02 (s, 1H), 7.07 (d, J=8.7 Hz, 1H), 7.39 (dd, J=4.5, 8.1 Hz, 1H), 7.80 (d, J=8.7 Hz, 1H), 8.62-8.63 (m, 1H), 9.04 (d, J=8.1 Hz, 1H), 14.39 (brs, 1H).

(c) Step 3

Two milliliters of trifluoroacetic acid was added to 8 mL of a methylene chloride solution of tert-butyl (S,Z)-[1-({2-[(1H-pyrazolo[3,4-b]pyridin-3-yl)methylene]-6-methoxy-3-oxo-2,3-dihydrobenzofuran-7-yl}methyl)pyrrolidin-3-yl]carbamate (0.148 g, 0.301 mmol), and stirred for 15 hours at room temperature. Eight milliliters of saturated sodium hydrogen carbonate was added to the residue obtained by concentrating the reaction solution, and the reaction system was extracted five times by chloroform. The organic layer was dried with anhydrous sodium sulfate, and the solvent was distilled off to obtain the target (S,Z)-2-[(1H-pyrazolo[3,4-b]pyridin-3-yl)methylene]-7-[(3-aminopyrrolidin-1-yl)methyl]-6-methoxybenzofuran-3(2H)-one (0.0751 g, 64%).

¹H NMR (300 MHz, DMSO-d₆) δ 1.32-1.42 (m, 1H), 1.95-2.07 (m, 1H), 2.24-2.29 (m, 1H), 2.59-2.68 (m, 2H), 2.78-2.83 (m, 1H), 3.31-3.39 (m, 1H), 3.83 (s, 2H), 3.98 (s, 3H), 7.04 (s, 1H), 7.07 (d, J=8.7 Hz, 1H), 7.37 (dd, J=5.1, 8.1 Hz, 1H), 7.79 (d, J=8.7 Hz, 1H), 8.62 (d, J=5.1 Hz, 1H), 9.05 (dd, J=1.5, 8.1 Hz, 1H).

### Synthesis Example 10

### Synthesis of compound 15 ((R,Z)-2-[(1H-pyrazolo[3,4-b]pyridin-3-yl)methylene]-7-[(3-aminopyrrolidin-1-yl)methyl]-6-methoxybenzofuran-3(2H)-one)

Compound 15 was synthesized by synthesis scheme 6 above.

(a) Step 1

The 7-(bromomethyl)-6-methoxybenzofuran-3(2H)-one (0.514 g, 2.00 mmol) described in [WO2011/136319] and potassium carbonate (0.276 g, 2.00 mmol) were added to 8 mL of methylene chloride, and stirred at room temperature. Four milliliters of a methylene chloride solution of tert-butyl (R)-pyrrolidin-3-ylcarbamate (0.373 g, 2.00 mmol) was added dropwise, and stirring was continued for 10 hours at room temperature. The reaction solution was filtered, and the residue obtained by concentrating the filtrate was purified by silica gel chromatography (methanol/chloroform) to obtain tert-butyl (R)-{1-[(6-methoxy-3-oxo-2,3-dihydrobenzofuran-7-yl)methyl]pyrrolidin-3-yl}carbamate (0.275 g, 38%).

¹H NMR (300 MHz, DMSO-d₆) δ 1.35 (s, 9H), 1.43-1.56 (m, 1H), 1.90-2.02 (m, 1H), 2.22-2.27 (m, 1H), 2.45-2.47 (m, 1H), 2.55 (m, 1H), 2.70-2.75 (m, 1H), 3.60 (s, 2H), 3.79-3.87 (m, 1H), 3.90 (s, 3H), 4.77 (s, 2H), 6.87-6.90 (m, 2H), 7.58 (d, J=8.7 Hz, 1H).

(b) Step 2

The 1H-pyrazolo[3,4-b]pyridine-3-carbaldehyde (0.0512 g, 0.348 mmol) described in [WO2011/136319] was added to 1.4 mL of a methanol solution of tert-butyl (R)-{1-[(6-methoxy-3-oxo-2,3-dihydrobenzofuran-7-yl)methyl]pyrrolidin-3-yl}carbamate (0.126 g, 0.348 mmol). After adding piperidine (0.0237 g, 0.278 mmol), the reaction system was stirred for two hours at 60° C. The residue obtained by concentrating the reaction solution was purified by silica gel chromatography (methanol/chloroform), and tert-butyl (R,Z)-[1-({2-[(1H-pyrazolo[3,4-b]pyridin-3-yl)methylene]-6-methoxy-3-oxo-2,3-dihydrobenzofuran-7-yl}methyl)pyrrolidin-3-yl]carbamate (0.113 g, 66%) was obtained.

¹H NMR (300 MHz, DMSO-d₆) δ 1.31-1.39 (m, 1H), 1.36 (s, 9H), 1.51-1.61 (m, 1H), 1.95-2.07 (m, 1H), 2.34-2.39 (m, 1H), 2.58-2.62 (m, 1H), 2.83-2.88 (m, 1H), 3.81 (s, 2H), 3.85-3.91 (m, 1H), 3.98 (s, 3H), 6.92 (d, J=6.6 Hz, 1H), 7.02 (s, 1H), 7.07 (d, J=8.7 Hz, 1H), 7.39 (dd, J=4.5, 8.1 Hz, 1H), 7.80 (d, J=8.7 Hz, 1H), 8.62-8.63 (m, 1H), 9.04 (d, J=8.1 Hz, 1H), 14.39 (brs, 1H).

(c) Step 3

Two milliliters of trifluoroacetic acid was added to 8 mL of a methylene chloride solution of tert-butyl (R,Z)-[1-({2-[(1H-pyrazolo[3,4-b]pyridin-3-yl)methylene]-6-methoxy-3-oxo-2,3-dihydrobenzofuran-7-yl}methyl)pyrrolidin-3-yl]carbamate (0.111 g, 0.226 mmol), and stirred for 15 hours at room temperature. The residue obtained by concentrating the reaction solution was made basic by adding saturated sodium hydrogen carbonate, and the reaction system was extracted five times by chloroform. The organic layer was dried with anhydrous sodium sulfate, and the solvent was distilled off to obtain the target (R,Z)-2-[(1H-pyrazolo[3,4-b]pyridin-3-yl)methylene]-7-[(3-aminopyrrolidin-1-yl)methyl]-6-methoxybenzofuran-3(2H)-one (0.0484 g, 55%).

¹H NMR (300 MHz, DMSO-d₆) δ 1.32-1.42 (m, 1H), 1.95-2.07 (m, 1H), 2.24-2.29 (m, 1H), 2.59-2.68 (m, 2H), 2.78-2.83 (m, 1H), 3.31-3.39 (m, 1H), 3.83 (s, 2H), 3.98 (s, 3H), 7.04 (s, 1H), 7.07 (d, J=8.7 Hz, 1H), 7.37 (dd, J=5.1, 8.1 Hz, 1H), 7.79 (d, J=8.7 Hz, 1H), 8.62 (d, J=5.1 Hz, 1H), 9.05 (dd, J=1.5, 8.1 Hz, 1H).

### Synthesis Example 11

### Synthesis of compound 16 ((Z)-6-methoxy-2-[(6-methyl-1H-pyrazolo[3,4-b]pyridin-3-yl)methylene]-7-(piperazin-4-ylmethyl)benzofuran-3(2H)-one)

Compound 16 was synthesized by synthesis scheme 7 below.

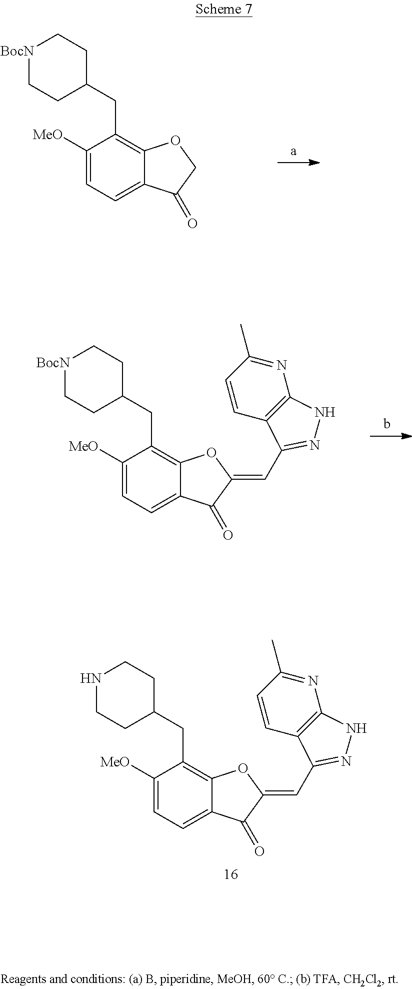 [SMILES: COc1ccc2c(c1CC1CCN(C(=O)OC(C)(C)C)CC1)OCC2=O ; COc1ccc2c(c1CC1CCN(C(=O)OC(C)(C)C)CC1)O/C(=C\c1n[nH]c3nc(C)ccc13)C2=O ; COc1ccc2c(c1CC1CCNCC1)O/C(=C\c1n[nH]c3nc(C)ccc13)C2=O]

(a) Step 1

6-Methyl-1H-pyrazolo[3,4-b]pyridine-3-carbaldehyde (0.161 g, 1.00 mmol) was added to 4 mL of a methanol solution of the tert-butyl 4-[(6-methoxy-3-oxo-2,3-dihydrobenzofuran-7-yl)methyl]piperidine-1-carbamate (0.361 g, 1.00 mmol) described in [WO2011/136319]. After adding piperidine (0.0681 g, 0.800 mmol), the reaction system was stirred for two hours at 60° C. The reaction solution was cooled to room temperature, and the precipitated solid was filtered out to obtain tert-butyl (Z)-4-({6-methoxy-2-[(6-methyl-1H-pyrazolo[3,4-b]pyridin-3-yl)methylene]-3-oxo-2,3-dihydrobenzofuran-7-yl}methyl)piperidine-1-carboxylate (0.452 g, 90%).

¹H NMR (300 MHz, DMSO-d₆) δ 1.07-1.20 (m, 2H), 1.33 (s, 9H), 1.58-1.62 (m, 2H), 1.78-1.90 (m, 1H), 2.56-2.66 (m, 2H), 2.61 (s, 3H), 2.74 (d, J=7.2 Hz, 2H), 3.87-3.92 (m, 2H), 3.97 (s, 3H), 6.95 (s, 1H), 7.03 (d, J=8.7 Hz, 1H), 7.18 (d, J=8.7 Hz, 1H), 7.71 (d, J=7.8 Hz, 1H), 8.73 (d, J=8.1 Hz, 1H), 13.99 (brs, 1H).

(b) Step 2

Eight milliliters of trifluoroacetic acid was added to 8 mL of a methylene chloride solution of tert-butyl (Z)-4-({6-methoxy-2-[(6-methyl-1H-pyrazolo[3,4-b]pyridin-3-yl)methylene]-3-oxo-2,3-dihydrobenzofuran-7-yl}methyl)piperidine-1-carboxylate (0.449 g, 0.890 mmol), and stirred for 16 hours at room temperature. The residue obtained by concentrating the reaction solution was made basic by adding saturated sodium hydrogen carbonate, and the precipitated solid was filtered out. After washing with water, the solid was dried under reduced pressure to obtain the target (Z)-6-methoxy-2-[(6-methyl-1H-pyrazolo[3,4-b]pyridin-3-yl)methylene]-7-(piperidin-4-ylmethyl)benzofuran-3(2H)-one (0.360 g, quantitative).

¹H NMR (300 MHz, DMSO-d₆) δ 1.32-1.46 (m, 2H), 1.71-1.76 (m, 2H), 1.90-1.99 (m, 1H), 2.64 (s, 3H), 2.68-2.79 (m, 4H), 3.17-3.21 (m, 2H), 3.97 (s, 3H), 6.99 (s, 1H), 7.06 (d, J=8.7 Hz, 1H), 7.27 (d, J=8.7 Hz, 1H), 7.74 (d, J=8.7 Hz, 1H), 8.73 (d, J=8.1 Hz, 1H), 13.99 (brs, 1H).

### Synthesis Example 12

### Synthesis of compound 17 ((Z)-2-[(1H-pyrazolo[3,4-b]pyridin-3-yl)methylene]-6-(difluoromethoxy)-7-(piperidin-4-ylmethyl)benzofuran-3(2H)-one)

Compound 17 was synthesized by synthesis scheme 8 below.

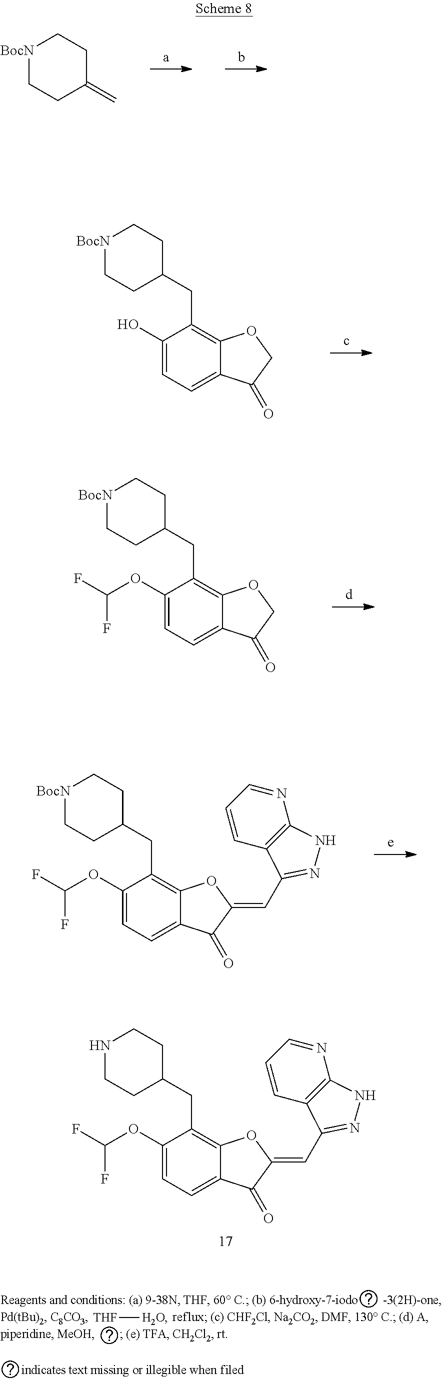

(a) Step 1

9-BBN (0.5 M tetrahydrofuran solution, 26.0 mL, 13.0 mmol) was added to 5 mL of a tetrahydrofuran solution of tert-butyl 4-methylenepiperidine-1-carboxylate (2.12 g, 10.8 mmol), and stirred for three hours at 60° C.

(b) Step 2

After cooling the reaction solution to room temperature, the 6-hydroxy-7-iodobenzofuran-3(2H)-one (3.00 g, 10.9 mmol) described in [WO2011-136319], cesium carbonate (7.04 g, 21.6 mmol), Pd(P^{t}Bu₃)₂ (0.277 g, 0.542 mmol), and water (15 mL) were added, and stirred for five hours at 60° C. After cooling to room temperature, the reaction solution was diluted by adding water (30 mL) and extracted three times with ethyl acetate. The organic layer was washed with (saturated) brine, dried with anhydrous sodium sulfate, and the solvent was distilled off. The residue obtained was purified by silica gel chromatography (ethyl acetate/petroleum ether), and tert-butyl 4-[(6-hydroxy-3-oxo-2,3-dihydrobenzofuran-7-yl)methyl]piperidine-1-carboxylate (2.50 g, 66%) was obtained.

¹H NMR (400 MHz, CDCl₃) δ 1.22-1.32 (m, 2H), 1.42-1.50 (m, 10H), 1.72-1.85 (m, 2H), 2.60-2.82 (m, 4H), 3.97-4.07 (m, 2H), 4.64 (s, 2H), 6.69 (d, J=8.8 Hz, 1H), 7.41 (d, J=8.4 Hz, 1H), 9.33 (brs, 1H).

(c) Step 3

CHF₂Cl was bubbled into 15 mL of a dimethylformamide solution of tert-butyl 4-[(6-hydroxy-3-oxo-2,3-dihydrobenzofuran-7-yl)methyl]piperidine-1-carboxylate (1.50 g, 4.32 mmol) and sodium carbonate (0.550 g, 5.19 mmol) over two hours at 130° C. After cooling to room temperature, the reaction solution was diluted by adding water (50 mL) and extracted three times with ethyl acetate. The organic layer was washed with (saturated) brine, dried with anhydrous sodium sulfate, and the solvent was distilled off. The residue obtained was purified by silica gel chromatography (ethyl acetate/petroleum ether) to obtain tert-butyl 4-{[6-(difluoromethoxy)-3-oxo-2,3-dihydrobenzofuran-7-yl]methyl}piperidine-1-carboxylate (0.700 g, 41%).

¹H NMR (400 MHz, CDCl₃) δ 1.20-1.25 (m, 2H), 1.45 (s, 9H), 1.58-1.62 (m, 2H), 1.70-1.80 (m, 1H), 2.58-2.68 (m, 4H), 4.00-4.13 (m, 2H), 4.67 (s, 2H), 6.61 (t, J=72.8 Hz, 1H), 6.83 (d, J=8.4 Hz, 1H), 7.55 (d, J=8.8 Hz, 1H).

(d) Step 4

The 1H-pyrazolo[3,4-b]pyridine-3-carbaldehyde (0.129 g, 0.878 mmol) described in [WO2011/136319] and piperidine (0.0570 g, 0.669 mmol) were added to 5 mL of a methanol solution of tert-butyl 4-{[6-(difluoromethoxy)-3-oxo-2,3-dihydrobenzofuran-7-yl]methyl}piperidine-1-carboxylate (0.332 g, 0.836 mmol), and tert-butyl (Z)-4-({2-[(1H-pyrazolo[3,4-b]pyridin-3-yl)methylene]-6-(difluoromethoxy)-3-oxo-2,3-dihydrobenzofuran-7-yl}methyl)piperidine-1-carboxylate (0.176 g, 40%) was obtained by the same procedure as in step 1 of Synthesis Example 1.

¹H NMR (400 MHz, CDCl₃) δ 1.30-1.40 (m, 2H), 1.45 (s, 9H), 1.69-1.76 (m, 2H), 1.89-2.02 (m, 1H), 2.68 (t, J=12.0 Hz, 2H), 2.93 (d, J=7.6 Hz, 2H), 3.98-4.25 (m, 2H), 6.70 (t, J=72.8 Hz, 1H), 7.04 (d, J=8.4 Hz, 1H), 7.27-7.35 (m, 2H), 7.78 (d, J=8.4 Hz, 1H), 8.70 (dd, J=1.2, 4.4 Hz, 1H), 8.89 (dd, J=1.2, 8.0 Hz, 1H), 11.82 (brs, 1H).

(e) Step 5

One milliliter of trifluoroacetic acid was added to 10 mL of a methylene chloride solution of tert-butyl (Z)-4-({2-[(1H-pyrazolo[3,4-b]pyridin-3-yl)methylene]-6-(difluoromethoxy)-3-oxo-2,3-dihydrobenzofuran-7-yl}methyl)piperidine-1-carboxylate (0.166 g, 0.315 mmol), and (Z)-2-[(1H-pyrazolo[3,4-b]pyridin-3-yl)methylene-6-(difluormethoxy)-7-(piperidin-4-ylmethyl)benzofuran-3(2H)-one (0.130 g, 97%) was obtained in the same way as in step 10 of Synthesis Example 2.

¹H NMR (400 MHz, DMSO-d₆) δ 1.32-1.46 (m, 2H), 1.70-1.76 (m, 2H), 1.90-2.00 (m, 1H), 2.69 (t, J=12.4 Hz, 2H), 2.82 (d, J=6.8 Hz, 2H), 3.17 (d, J=12.4 Hz, 2H), 7.11-7.15 (m, 2H), 7.40 (dd, J=4.4, 8.0 Hz, 1H), 7.49 (t, J=73.2 Hz, 1H), 7.81 (d, J=8.4 Hz, 1H), 8.64 (dd, J=1.6, 4.8 Hz, 1H), 8.84 (dd, J=1.6, 8.4 Hz, 1H).

### Synthesis Example 13

### Synthesis of compound 18 ((Z)-2-[(1H-pyrazolo[3,4-b]pyridin-3-yl)methylene]-7-[(3-fluoropiperidin-4-yl)methyl]-6-methoxybenzofuran-3(2H)-one (cis isomer)

Compound 18 was synthesized by synthesis scheme 9 below.

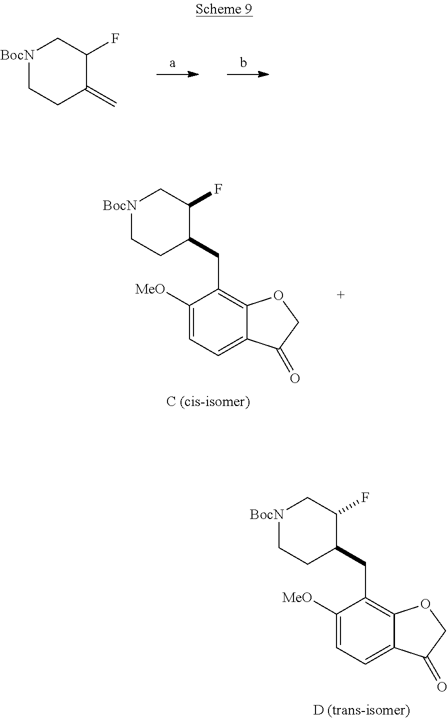

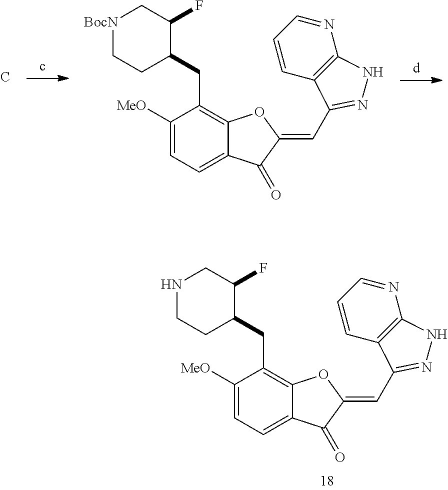

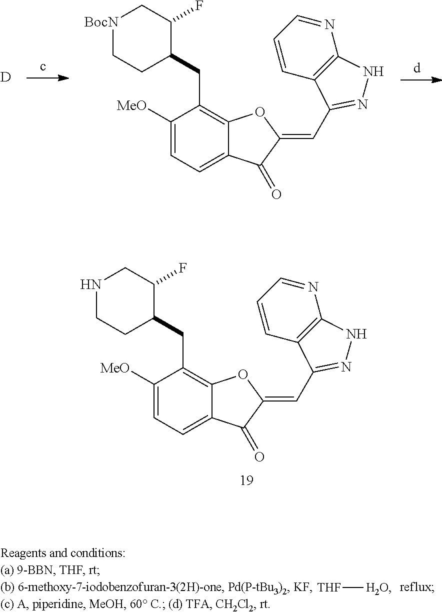

(a) Step 1

9-BBN (0.5 M tetrahydrofuran solution, 46.4 mL, 23.2 mmol) was added to 20 mL of a tetrahydrofuran solution of the tert-butyl 3-fluoro-4-methylenepiperidine-1-carboxylate (5.00 g, 23.2 mmol) described in the known literature [European Journal of Medicinal Chemistry, vol. 53, pp. 408-415, 2012], and stirred for three hours at room temperature.

(b) Step 2

The 6-methoxy-7-iodobenzofuran-3(2H)-one (6.74 g, 23.2 mmol) described in [WO2011/136319] and potassium fluoride (4.03 g, 69.4 mmol) were dissolved in a tetrahydrofuran/water mixed solvent (40 mL/50 mL), and added to the above reaction solution. Next, Pd(P^{t}Bu₃)₂ (0.590 g, 1.15 mmol) was added, and heated and refluxed for 18 hours. After cooling to room temperature, the reaction solution was diluted by adding water and extracted three times with ethyl acetate (100 mL). The organic layer was washed with (saturated) brine (100 mL), dried with anhydrous sodium sulfate, and the solvent was distilled off. The residue obtained was purified by silica gel chromatography (ethyl acetate/petroleum ether) to obtain a cis isomer (C) (2.20 g, 25%) and trans isomer (D) (2.90 g, 33%) of tert-butyl 3-fluoro-4-[(6-methoxy-3-oxo-2,3-dihydrobenzofuran-7-yl)methyl]piperidine-1-carboxylate.

D was confirmed to be a trans isomer by two-dimensional NMR.

¹H NMR (cis isomer) (400 MHz, CD₃CN) δ 1.30-1.40 (m, 1H), 1.44 (s, 9H), 1.52-1.62 (m, 1H), 1.80-1.83 (m, 1H), 2.60-2.90 (m, 4H), 3.93 (s, 3H), 4.02-4.10 (m, 1H), 4.25-4.38 (m, 1H), 4.50-4.67 (m, 3H), 6.83 (d, J=8.8 Hz, 1H), 7.53 (d, J=8.4 Hz, 1H).

¹H NMR (trans isomer) (400 MHz, CD₃CN) δ 1.20-1.30 (m, 1H), 1.45 (s, 9H), 1.55-1.65 (m, 1H), 2.00-2.10 (m, 1H), 2.60-2.68 (m, 1H), 2.70-2.80 (m, 1H), 2.95-3.10 (m, 2H), 3.70-3.78 (m, 1H), 3.93 (s, 3H), 4.02-4.12 (m, 1H), 4.21-4.40 (m, 1H), 4.63 (s, 2H), 6.82 (d, J=8.8 Hz, 1H), 7.54 (d, J=8.8 Hz, 1H).

(c) Step 3

The 1H-pyrazolo[3,4-b]pyridine-3-carbaldehyde (0.256 g, 1.74 mmol) described in [WO2011/136319] and piperidine (0.108 g, 1.27 mmol) were added to 10 mL of a methanol solution of tert-butyl 3-fluoro-4-[(6-methoxy-3-oxo-2,3-dihydrobenzofuran-7-yl)methyl]piperidine-1-carboxylate (cis isomer) (0.600 g, 1.58 mmol), and the same procedure as in step 1 of Synthesis Example 1 was conducted. tert-Butyl 4-({(Z)-2-[(1-H-pyrazolo[3,4-b]pyridin-3-yl)methylene]-6-methoxy-3-oxo-2,3-dihydrobenzofuran-7-yl}methyl)-3-fluoropiperidine-1-carboxylate (cis isomer) (0.660 g, 82%) was obtained.

¹H NMR (400 MHz, CDCl₃) δ 1.45 (s, 10H), 1.70-1.90 (m, 1H), 2.00-2.20 (m, 1H), 2.60-3.12 (m, 4H), 4.02 (s, 3H), 4.04-4.80 (m, 3H), 6.85 (d, J=8.8 Hz, 1H), 7.29 (s, 1H), 7.34 (m, 1H), 7.79 (d, J=8.4 Hz, 1H), 8.65 (dd, J=1.2, 4.4 Hz, 1H), 9.05 (d, J=7.6 Hz, 1H), 11.59 (brs, 1H).

(d) Step 4

One milliliter of trifluoroacetic acid was added to 15 mL of a methylene chloride solution of tert-butyl 4-({(Z)-2-[(1-H-pyrazolo[3,4-b]pyridin-3-yl)methylene]-6-methoxy-3-oxo-2,3-dihydrobenzofuran-7-yl}methyl)-3-fluoropiperidine-1-carboxylate (cis isomer) (0.660 g, 1.30 mmol), and the same procedure as in step 10 of Synthesis Example 2 was conducted. The target (Z)-2-[(1H-pyrazolo[3,4-b]pyridin-3-yl)methylene]-7-[(3-fluoropiperidin-4-yl)methyl]-6-methoxybenzofuran-3(2H)-one (cis isomer) (0.325 g, 61%) was obtained.

¹H NMR (400 MHz, DMSO-d₆) δ 1.25-1.32 (m, 1H), 1.44-1.57 (m, 1H), 1.95-2.12 (m, 1H), 2.37-2.41 (m, 1H), 2.55-2.70 (m, 1H), 2.82-2.98 (m, 3H), 3.08-3.14 (m, 1H), 3.98 (s, 3H), 4.54 (d, J=48.4 Hz, 1H), 7.02 (s, 1H), 7.06 (d, J=8.8 Hz, 1H), 7.36 (dd, J=4.4, 8.4 Hz, 1H), 7.75 (d, J=8.8 Hz, 1H), 8.64 (dd, J=1.6, 4.4 Hz, 1H), 8.98 (dd, J=1.2, 8.0 Hz, 1H).

### Synthesis Example 14

### Synthesis of compound 19 ((Z)-2-[(1H-pyrazolo[3,4-b]pyridin-3-yl)methylene]-7-[(3-fluoropiperidin-4-yl)methyl]-6-methoxybenzofuran-3(2H)-one (trans isomer)

(c) Step 3

The 1H-pyrazolo[3,4-b]pyridine-3-carbaldehyde (0.294 g, 2.00 mmol) described in [WO2011/136319] and piperidine (0.125 g, 1.47 mmol) were added to 16 mL of a methanol solution of the tert-butyl 3-fluoro-4-[(6-methoxy-3-oxo-2,3-dihydrobenzofuran-7-yl)methyl]piperidine-1pcarboxylate (trans isomer) (0.700 g, 1.84 mmol) obtained in step 2 of Synthesis Example 13. The same procedure as in step 1 of Synthesis Example 1 was conducted, and tert-butyl 4-({(Z)-2-[(1H-pyrazolo[3,4-b]pyridin-3-yl)methylene]-6-methoxy-3-oxo-2,3-dihydrobenzofuran-7-yl}methyl)-3-fluoropiperidine-1-carboxylate (trans isomer) (0.540 g, 57%) was obtained.

¹H NMR (400 MHz, CDCl₃) δ 1.00-1.70 (m, 12H), 2.05-2.20 (m, 1H), 2.66 (t, J=12.0 Hz, 1H), 2.70-2.95 (m, 2H), 3.38 (dd, J=4.8, 13.6 Hz, 1H), 3.98 (s, 3H), 4.25-4.50 (m, 2H), 6.82 (d, J=8.8 Hz, 1H), 7.27 (s, 1H), 7.35 (dd, J=4.4, 8.0 Hz, 1H), 7.75 (d, J=8.4 Hz, 1H), 8.68 (dd, J=1.2, 4.4 Hz, 1H), 8.90-9.00 (m, 1H), 12.55 (brs, 1H).

(d) Step 4

Two milliliters of trifluoroacetic acid was added to 20 mL of a methylene chloride solution of tert-butyl 4-({(Z)-2-[(1H-pyrazolo[3,4-b]pyridin-3-yl)methylene]-6-methoxy-3-oxo-2,3-dihydrobenzofuran-7-yl}methyl)-3-fluoropiperidine-1-carboxylate (trans isomer) (0.540 g, 1.06 mmol). The same procedure as in step 10 of Synthesis Example 2 was conducted, and the target (Z)-2-[(1H-pyrazolo[3,4-b]pyridin-3-yl)methylene]-7-[(3-fluoropiperidin-4-yl)methyl]-6-methoxybenzofuran-3(2H)-one (trans isomer) (0.370 g, 85%) was obtained.

¹H NMR (400 MHz, DMSO-d₆) δ 1.10-1.25 (m, 1H), 1.40-1.50 (m, 1H), 1.90-2.00 (m, 1H), 2.15-2.25 (m, 1H), 2.30-2.40 (m, 1H), 2.65-2.80 (m, 2H), 3.10-3.30 (m, 2H), 3.96 (s, 3H), 4.20-4.40 (m, 1H), 7.02 (s, 1H), 7.05 (d, J=8.8 Hz, 1H), 7.36 (dd, J=4.4, 8.0 Hz, 1H), 7.72 (d, J=8.8 Hz, 1H), 8.68 (dd, J=1.2, 4.4 Hz, 1H), 8.92 (dd, J=1.2, 8.0 Hz, 1H).

### Synthesis Example 15

### Synthesis of compound 22 ((Z)-2-[(1H-pyrazolo[3,4-b]pyridin-3-yl)methylene]-6-ethoxy-7-[1-(piperazin-1-yl)ethyl]benzofuran-3(2H)-one)

Compound 22 was synthesized by synthesis scheme 10 below.

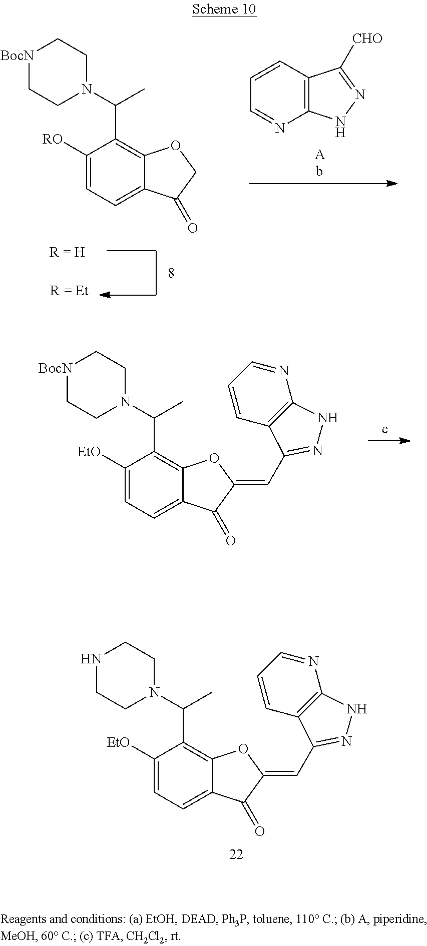

(a) Step 1

Ethanol (138 mg, 3.00 mmol), triphenyl phosphine (0.984 g, 3.75 mmol), and 40% diethyl azodicarboxylate/toluene solution (1.63 g, 3.75 mmol) were added to 10 mL of a toluene solution of the tert-butyl 4-[1-(6-hydroxy-3-oxo-2,3-dihydrobenzofuran-7-yl)ethyl]piperazin-1-carboxylate (0.906 g, 2.50 mmol) described in [WO2011/136319], and stirred for five hours at 110° C. The reaction solution was concentrated, and the crude product obtained by subjecting the residue obtained to silica gel chromatography (ethyl acetate/chloroform) was further purified by silica gel chromatography (ethyl acetate/hexane) to obtain tert-butyl 4-[1-(6-ethoxy-3-oxo-2,3-dihydrobenzofuran-7-yl)ethyl]piperazine-1-carboxylate (0.608 g, 62%).

¹H NMR (300 MHz, CDCl₃) δ 1.42 (s, 9H), 1.46 (t, J=6.6 Hz, 3H), 1.57 (d, J=7.2 Hz, 3H), 2.39-2.46 (m, 4H), 3.37-3.47 (m, 4H), 4.13 (m, 2H), 4.34 (q, J=7.2 Hz, 1H), 4.61 (s, 2H), 6.67 (d, J=8.1 Hz, 1H), 7.57 (d, J=8.1 Hz, 1H).

(b) Step 2

The 1H-pyrazolo[3,4-b]pyridine-3-carbaldehyde (0.0837 g, 0.569 mmol) described in [WO2011/136319] and piperidine (0.0387 g, 0.455 mmol) were added to 2.5 mL of a methanol solution of tert-butyl 4-[1-(6-ethoxy-3-oxo-2,3-dihydrobenzofuran-7-yl)ethyl]piperazine-1-carboxylate (0.222 g, 0.569 mmol), and stirred for two hours at 60° C. The reaction solution was cooled to room temperature, and the precipitated solid was filtered out to obtain tert-butyl (Z)-4-(1-{2-[(1H-pyrazolo[3,4-b]pyridin-3-yl)methylene]-6-ethoxy-3-oxo-2,3-dihydrobenzofuran-7-yl}ethyl)piperazine-1-carboxylate (0.215 g, 72%).

¹H NMR (300 MHz, DMSO-d₆) δ 1.34 (s, 9H), 1.40 (t, J=6.6 Hz, 3H), 1.57 (d, J=6.6 Hz, 3H), 2.38-2.59 (m, 4H), 3.29-3.32 (m, 4H), 4.18 (q, J=6.6 Hz, 2H), 4.24 (q, J=6.6 Hz, 1H), 7.04 (d, J=8.7 Hz, 1H), 7.07 (s, 1H), 7.36 (dd, J=4.5, 8.1 Hz, 1H), 7.75 (d, J=8.7 Hz, 1H), 8.64 (dd, J=1.5, 4.5 Hz, 1H), 9.26 (d, J=8.1 Hz, 1H), 14.35 (brs, 1H).

(c) Step 3

Six milliliters of trifluoroacetic acid was added at room temperature to 6 mL of a methylene chloride solution of tert-butyl (Z)-4-(1-{2-[(1H-pyrazolo[3,4-b]pyridin-3-yl)methylene]-6-ethoxy-3-oxo-2,3-dihydrobenzofuran-7-yl}ethyl)piperazine-1-carboxylate (0.179 g, 0.345 mmol), and stirred for 14 hours at room temperature. After distilling off the solvent, the residue obtained was made basic by adding 6 mL of saturated sodium hydrogen carbonate aqueous solution, and the precipitated solid was filtered out. The solid obtained was washed with water and dried under reduced pressure to obtain the target (Z)-2-[(1H-pyrazolo[3,4-b]pyridin-3-yl)methylene]-6-ethoxy-7-[1-(piperazin-1-yl)ethyl]benzofuran-3(2H)-one (0.140 g, 96%).

¹H NMR (300 MHz, DMSO-d₆) δ 1.40 (t, J=6.6 Hz, 3H), 1.55 (d, J=7.2 Hz, 3H), 2.45 (m, 2H), 2.62 (m, 2H), 2.85 (m, 4H), 4.16 (q, J=7.2 Hz, 1H), 4.24 (q, J=6.6 Hz, 2H), 7.05 (d, J=8.7 Hz, 1H), 7.07 (s, 1H), 7.34 (dd, J=4.5, 8.1 Hz, 1H), 7.76 (d, J=8.7 Hz, 1H), 8.64 (dd, J=1.5, 4.5 Hz, 1H), 9.29 (d, J=8.1 Hz, 1H).

### Synthesis Example 16

### Synthesis of compound 23 ((Z)-2-[(1H-pyrazolo[3,4-b]pyridin-3-yl)methylene]-6-(cyclopropylmethoxy)-7-[1-(piperazin-1-yl)ethyl]benzofuran-3(2H)-one)

Compound 23 was synthesized by synthesis scheme 11 below.

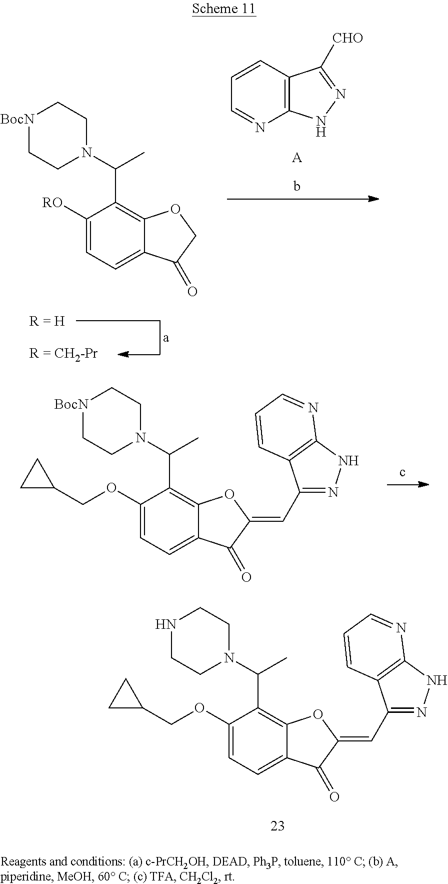

(a) Step 1

Cyclopropylmethanol (216 mg, 3.00 mmol), triphenyl phosphine (0.984 g, 3.75 mmol), and 40% diethyl azodicarboxylate/toluene solution (1.63 g, 3.75 mmol) were added to 10 mL of a toluene solution of the tert-butyl 4-[1-(6-hydroxy-3-oxo-2,3-dihydrobenzofuran-7-yl)ethyl]piperazine-1-carboxylate (0.906 g, 2.50 mmol) described in [WO2011/136319], and stirred for five hours at 110° C. The reaction solution was concentrated, and the crude product obtained by subjecting the residue obtained to silica gel chromatography (ethyl acetate/chloroform) was further purified by silica gel chromatography (ethyl acetate/hexane) to obtain tert-butyl 4-{1-[6-(cyclopropylmethoxy)-3-oxo-2,3-dihydrobenzofuran-7-yl]ethyl}piperazin-1-carboxylate (0.576 g, 55%).

¹H NMR (300 MHz, CDCl₃) δ 0.33-0.38 (m, 2H), 0.64-0.70 (m, 2H), 1.28 (m, 1H), 1.42 (s, 9H), 1.59 (d, J=7.2 Hz, 3H), 4.08 (m, 4H), 3.41 (m, 4H), 3.90 (m, 2H), 4.38 (q, J=7.2 Hz, 1H), 4.61 (s, 2H), 6.62 (d, J=8.7 Hz, 1H), 7.56 (d, J=8.7 Hz, 1H).

(b) Step 2

The 1H-pyrazolo[3,4-b]pyridine-3-carbaldehyde (0.100 g, 0.682 mmol) described in [WO2011/136319] and piperidine (0.0465 g, 0.546 mmol) were added to 3 mL of a methanol solution of tert-butyl 4-{1-[6-(cyclopropylmethoxy)-3-oxo-2,3-dihydrobenzofuran-7-yl]ethyl}piperazin-1-carboxylate (0.284 g, 0.682 mmol), and stirred for two hours at 60° C. The reaction solution was cooled to room temperature, and the precipitated solid was filtered out to obtain tert-butyl (Z)-4-(1-{2-[(1H-pyrazolo[3,4-b]pyridin-3-yl)methylene]-6-(cyclopropylmethoxy)-3-oxo-2,3-dihydrobenzofuran-7-yl}ethyl)piperazine-1-carboxylate (0.215 g, 57%).

¹H NMR (300 MHz, DMSO-d₆) δ 0.35-0.40 (m, 2H), 0.58-0.64 (m, 2H), 1.34 (m, 10H), 1.59 (d, J=6.6 Hz, 3H), 2.40-2.46 (m, 2H), 2.54-2.60 (m, 2H), 3.31 (m, 4H), 4.01 (dd, J=6.6, 10.2 Hz, 1H), 4.10 (dd, J=6.6, 10.2 Hz, 1H), 4.22 (q, J=6.6 Hz, 1H), 7.02 (d, J=8.7 Hz, 1H), 7.07 (s, 1H), 7.35 (dd, J=4.5, 8.1 Hz, 1H), 7.73 (d, J=8.7 Hz, 1H), 8.63 (dd, J=1.5, 4.5 Hz, 1H), 9.24 (d, J=8.1 Hz, 1H), 14.37 (brs, 1H).

(c) Step 3

Six milliliters of trifluoroacetic acid was added at room temperature to 6 mL of a methylene chloride solution of tert-butyl (Z)-4-(1-{2-[(1H-pyrazolo[3,4-b]pyridin-3-yl)methylene]-6-(cyclopropylmethoxy)-3-oxo-2,3-dihydrobenzofuran-7-yl}ethyl)piperazine-1-carboxylate (0.188 g, 0.345 mmol), and stirred for 14 hours at room temperature. After distilling off the solvent, the residue obtained was made basic by adding 6 mL of saturated sodium hydrogen carbonate aqueous solution, and the precipitated solid was filtered out. The solid obtained was washed with water and dried under reduced pressure to obtain the target (Z)-2-[(1H-pyrazolo[3,4-b]pyridin-3-yl)methylene]-6-(cyclopropylmethoxy)-7-[1-(piperazin-1-yl)ethyl]benzofuran-3(2H)-one (0.150 g, 98%).

¹H NMR (300 MHz, DMSO-d₆) δ 0.37-0.41 (m, 2H), 0.61-0.64 (m, 2H), 1.30 (m, 1H), 1.59 (d, J=6.6 Hz, 3H), 2.55-2.59 (m, 2H), 2.66-2.74 (m, 2H), 2.94 (m, 4H), 4.02 (dd, J=6.6, 10.5 Hz, 1H), 4.10 (dd, J=6.6, 10.5 Hz, 1H), 4.24 (q, J=6.6 Hz, 1H), 7.03 (d, J=8.7 Hz, 1H), 7.09 (s, 1H), 7.36 (dd, J=4.2, 8.1 Hz, 1H), 7.75 (d, J=8.7 Hz, 1H), 8.64 (dd, J=1.5, 4.2 Hz, 1H), 9.22 (d, J=8.1 Hz, 1H).

### Synthesis Example 17

### Synthesis of compound 24 ((Z)-2-[(1H-pyrazolo[3,4-b]pyridin-3-yl)methylene]-6-methoxy-7-[3-(piperazin-1-yl)prop-1-en-2-yl]benzofuran-3(2H)-one)

Compound 24 was synthesized by synthesis scheme 12 below.

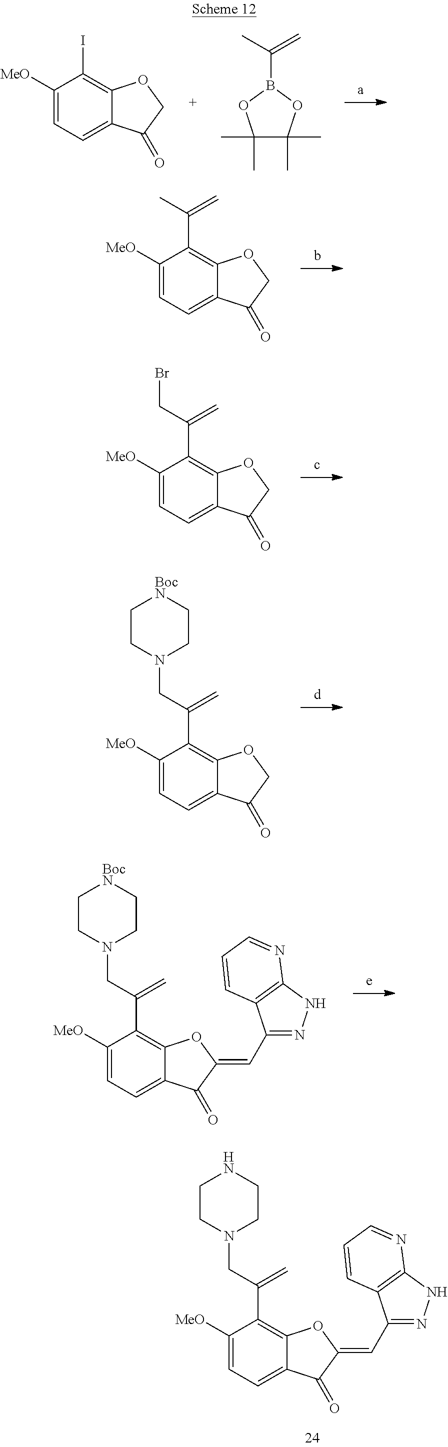

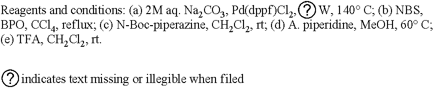

(a) Step 1

The 7-iodo-6-methoxybenzofuran-3(2H)-one (1.16 g, 4.00 mmol) described in [WO2011/136319] was added to 12 mL of 1,4-dioxane. Four milliliters of 2 M sodium carbonate aqueous solution, 4,4,5,5-tetramethyl-2-(prop-1-en-2-yl)-1,3,2-dioxaborolane (1.01 g, 6.00 mmol), and [1,1′-bis(diphenylphosphino)ferrocene]dichloropalladium(II) (0.293 g, 0.400 mmol) were also added, and the reaction system was heated for 10 minutes at 140° C. by a microwave irradiator (Initiator manufactured by Biotage). The reaction solution was poured into 20 mL of water, and 20 mL of (saturated) brine was added. The reaction solution was extracted three times with ethyl acetate, and the organic layer was dried by anhydrous magnesium sulfate. The residue obtained by distilling off the solvent was purified by silica gel column chromatography (ethyl acetate/hexane), and 6-methoxy-7-(prop-1-en-2-yl)benzofuran-3(2H)-one (0.401 g, 49%) was obtained.

¹H NMR (300 MHz, CDCl₃) δ 2.09 (s, 3H), 3.93 (s, 3H), 4.63 (s, 2H), 5.09 (d, J=1.5 Hz, 1H), 5.43 (d, J=1.5 Hz, 1H), 6.71 (d, J=8.7 Hz, 1H), 7.60 (d, J=8.7 Hz, 1H).

(b) Step 2

N-bromosuccinimide (NBS) (0.0979 g, 0.550 mmol) and benzoyl peroxide (BPO) (0.00608 g, 0.0250 mmol) were added to 5 mL of a carbon tetrachloride solution of methoxy-7-(prop-1-en-2-yl)benzofuran-3(2H)-one (0.102 g, 0.500 mmol), and heated to reflux for four hours. The reaction solution was filtered by Celite, and the filtrate was concentrated. The residue obtained was purified by silica gel column chromatography (ethyl acetate/hexane), and 7-(3-bromoprop-1-en-2-yl)-6-methoxybenzofuran-3(2H)-one (0.0490 g, 34%) was obtained.

¹H NMR (300 MHz, CDCl₃) δ 3.95 (s, 3H), 4.39 (s, 2H), 4.66 (s, 2H), 5.39 (s, 1H), 5.78 (s, 1H), 6.74 (d, J=8.7 Hz, 1H), 7.66 (d, J=8.7 Hz, 1H).

(c) Step 3

N-Boc-piperazine (0.0618 g, 0.332 mmol) was added to 2 mL of a methylene chloride solution of 7-(3-bromoprop-1-en-2-yl)-6-methoxybenzofuran-3(2H)-one (0.0448 g, 0.158 mmol), and stirred for three days at room temperature. The residue obtained by distilling off the solvent was purified by silica gel column chromatography (ethyl acetate/hexane), and tert-butyl 4-[2-(6-methoxy-3-oxo-2,3-dihydrobenzofuran-7-yl)allyl]piperazine-1-carboxylate (0.0518 g, 84%) was obtained.

¹H NMR (300 MHz, CDCl₃) δ 1.45 (s, 9H), 2.42 (t, J=5.1 Hz, 4H), 3.27 (s, 2H), 3.34 (t, J=5.1 Hz, 4H), 3.90 (s, 3H), 4.60 (s, 2H), 5.26 (d, J=1.5 Hz, 1H), 5.62 (d, J=1.5 Hz, 1H), 6.70 (d, J=8.7 Hz, 1H), 7.60 (d, J=8.7 Hz, 1H).

(d) Step 4

The 1H-pyrazolo[3,4-b]pyridin-3-carbaldehyde (0.00734 g, 0.0499 mmol) described in [WO2011/136319] and piperidine (0.00340 g, 0.0399 mmol) were added to 1 mL of a methanol solution of tert-butyl 4-[2-(6-methoxy-3-oxo-2,3-dihydrobenzofuran-7-yl)allyl]piperazine-1-carboxylate (0.0194 g, 0.0499 mmol), and stirred for two hours at 60° C. The residue obtained by distilling off the solvent was purified by silica gel column chromatography (methanol/chloroform), and tert-butyl (Z)-4-(2-{2-[(1H-pyrazolo[3,4-b]pyridin-3-yl)methylene]-6-methoxy-3-oxo-2,3-dihydrobenzofuran-7-yl}allyl)piperazine-1-carboxylate (0.0240 g, 92%) was obtained.

¹H NMR (300 MHz, CDCl₃) δ 1.40 (s, 9H), 2.45 (m, 4H), 3.27 (m, 4H), 3.35 (s, 2H), 3.96 (s, 3H), 5.47 (s, 1H), 5.87 (s, 1H), 6.83 (d, J=8.7 Hz, 1H), 7.22 (dd, J=4.5, 8.1 Hz, 1H), 7.23 (s, 1H), 7.78 (d, J=8.7 Hz, 1H), 8.63 (dd, J=1.5, 4.5 Hz, 1H), 8.95 (d, J=8.1 Hz, 1H), 11.50 (brs, 1H).

(e) Step 5

One milliliter of trifluoroacetic acid was added to 1 mL of a methylene chloride solution of tert-butyl (Z)-4-(2-{2-[(1H-pyrazolo[3,4-b]pyridin-3-yl)methylene]-6-methoxy-3-oxo-2,3-dihydrobenzofuran-7-yl}allyl)piperazine-1-carboxylate (0.0240 g, 0.0464 mmol), and stirred for 14 hours at room temperature. The residue obtained by concentrating the reaction solution was made basic by adding saturated sodium hydrogen carbonate aqueous solution, and the precipitated solid was filtered out. The solid filtered out was washed with water and dried under reduced pressure to obtain the target (Z)-2-[(1H-pyrazolo[3,4-b]pyridin-3-yl)methylene]-6-methoxy-7-[3-(piperazin-1-yl)prop-1-en-2-yl]benzofuran-3(2H)-one (0.00920 g, 47%).

¹H NMR (300 MHz, DMSO-d₆) δ 2.32 (m, 4H), 2.46 (m, 4H), 3.25 (s, 2H), 3.94 (s, 3H), 5.43 (s, 1H), 5.82 (s, 1H), 7.03 (s, 1H), 7.06 (d, J=8.7 Hz, 1H), 7.28 (dd, J=4.2, 8.7 Hz, 1H), 7.79 (d, J=8.7 Hz, 1H), 8.61 (d, J=4.2 Hz, 1H), 8.92 (d, J=8.7 Hz, 1H).

### Synthesis Example 18

### Synthesis of compound 25 ((Z)-2-[(1H-pyrazolo[3,4-b]pyridin-3-yl)methylene]-7-[(3,8-diazabicyclo[3.2.1]octan-3-yl)methyl]-6-methoxybenzofuran-3(2H)-one

Compound 25 was synthesized by synthesis scheme 13 below.

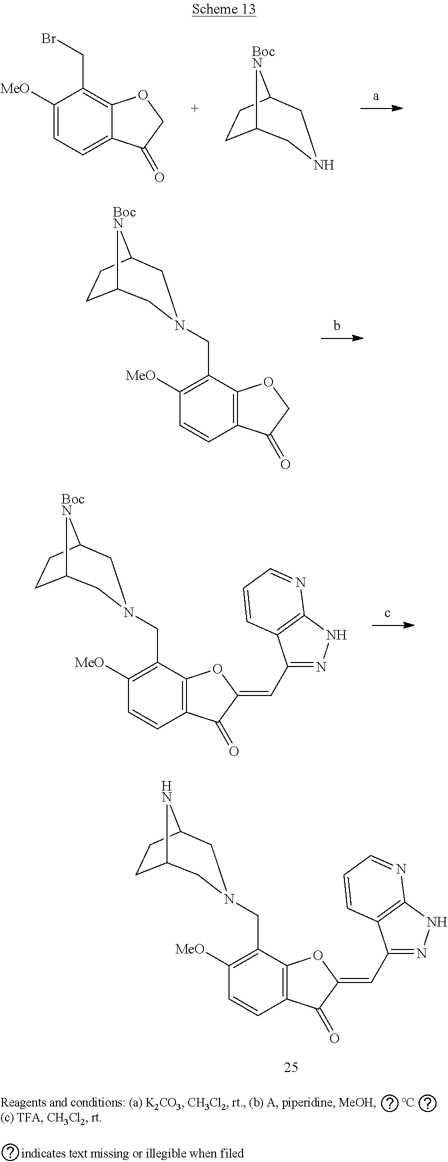 [SMILES: COc1ccc2c(c1CBr)OCC2=O ; CC(C)(C)OC(=O)N1C2CCC1CNC2 ; COc1ccc2c(c1CN1CC3CCC(C1)N3C(=O)OC(C)(C)C)OCC2=O ; COc1ccc2c(c1CN1CC3CCC(C1)N3C(=O)OC(C)(C)C)O/C(=C\c1n[nH]c3ncccc13)C2=O ; COc1ccc2c(c1CN1CC3CCC(C1)N3)O/C(=C\c1n[nH]c3ncccc13)C2=O]

(a) Step 1

Potassium carbonate (0.163 g, 1.18 mmol) was added to 5 mL of a methylene chloride solution of the 7-(bromomethyl)-6-methoxybenzofuran-3(2H)-one (0.303 g, 1.18 mmol) described in [WO2011/136319]. Two milliliters of a methylene chloride solution of 8-Boc-3,8-diazabicyclo[3.2.1]octane (0.250 g, 1.18 mmol) was added dropwise to the reaction solution, and stirring was continued for 15 hours at room temperature. The reaction solution was filtered, and the residue obtained by concentrating the filtrate was purified by silica gel chromatography (ethyl acetate/hexane) to obtain tert-butyl 3-[(6-methoxy-3-oxo-2,3-dihydrobenzofuran-7-yl)methyl]-3,8-diazabicyclo[3.2.1]octane-8-carboxylate (0.182 g, 39%).

¹H NMR (300 MHz, CDCl₃) δ 1.43 (s, 9H), 1.77 (m, 4H), 2.37 (m, 2H), 2.60 (dd, J=2.1, 11.1 Hz, 2H), 3.65 (s, 2H), 3.91 (s, 3H), 4.08-4.16 (m, 2H), 4.61 (s, 2H), 6.68 (d, J=9.0 Hz, 1H), 7.59 (d, J=9.0 Hz, 1H).

(b) Step 2

The 1H-pyrazolo[3,4-b]pyridine-3-carbaldehyde (0.0303 g, 0.206 mmol) described in [WO2011/136319] and piperidine (0.0140 g, 0.164 mmol) were added to 0.8 mL of a methanol solution of tert-butyl 3-[(6-methoxy-3-oxo-2,3-dihydrobenzofuran-7-yl)methyl]-3,8-diazabicyclo[3.2.1]octane-8-carboxylate (0.0800 g, 0.206 mmol), and stirred for two hours at 60° C. The reaction solution was cooled to room temperature, the precipitated solid was filtered out, and tert-butyl (Z)-3-({2-[(1H-pyrazolo[3,4-b]pyridin-3-yl)methylene]-6-methoxy-3-oxo-2,3-dihydrobenzofuran-7-yl}methyl)-3,8-diazabicyclo[3.2.1]octane-8-carboxylate (0.0613 g, 57%) was obtained.

¹H NMR (300 MHz, DMSO-d₆) δ 1.34 (s, 9H), 1.56 (m, 4H), 2.28 (d, J=10.2 Hz, 2H), 2.63-2.67 (m, 2H), 3.75 (s, 2H), 3.98 (s, 5H), 7.03 (s, 1H), 7.07 (d, J=8.7 Hz, 1H), 7.39 (dd, J=4.2, 8.1 Hz, 1H), 7.80 (d, J=8.7 Hz, 1H), 8.63 (dd, J=1.5, 4.2 Hz, 1H), 8.95 (dd, J=1.5, 8.1 Hz, 1H), 14.38 (brs, 1H).

(c) Step 3

Two milliliters of trifluoroacetic acid was added to 4 mL of a methylene chloride solution of tert-butyl (Z)-3-({2-[(1H-pyrazolo[3,4-b]pyridin-3-yl)methylene]-6-methoxy-3-oxo-2,3-dihydrobenzofuran-7-yl}methyl)-3,8-diazabicyclo[3.2.1]octane-8-carboxylate (0.0596 g, 0.115 mmol), and stirred for 16 hours at room temperature. The residue obtained by concentrating the reaction solution was made basic by adding 6 mL of saturated sodium hydrogen carbonate aqueous solution, and the precipitated solid was filtered out. The solid filtered out was washed with water and dried under reduced pressure to obtain the target (Z)-2-[(1H-pyrazolo[3,4-b]pyridin-3-yl)methylene]-7-[(3,8-diazabicyclo[3.2.1]octan-3-yl)methyl]-6-methoxybenzofuran-3(2H)-one (0.0270 g, 56%).

¹H NMR (300 MHz, DMSO-d₆) δ 1.51-1.63 (m, 4H), 2.40 (d, J=11.7 Hz, 2H), 2.66-2.70 (m, 2H), 3.55 (m, 2H), 3.79 (s, 2H), 3.97 (s, 3H), 7.05 (s, 1H), 7.08 (d, J=8.7 Hz, 1H), 7.40 (dd, J=4.5, 8.7 Hz, 1H), 7.81 (d, J=8.7 Hz, 1H), 8.65 (dd, J=1.5, 4.5 Hz, 1H), 8.95 (dd, J=1.5, 8.1 Hz, 1H).

### Synthesis Example 19

### Synthesis of compound 26 ((S,Z)-2-[(1H-pyrazolo[3,4-b]pyridin-3-yl)methylene]-6-methoxy-7-{[3-(methylamino)pyrrolidin-1-yl]methyl}benzofuran-3(2H)-one)

Compound 26 was synthesized by synthesis scheme 14 below.

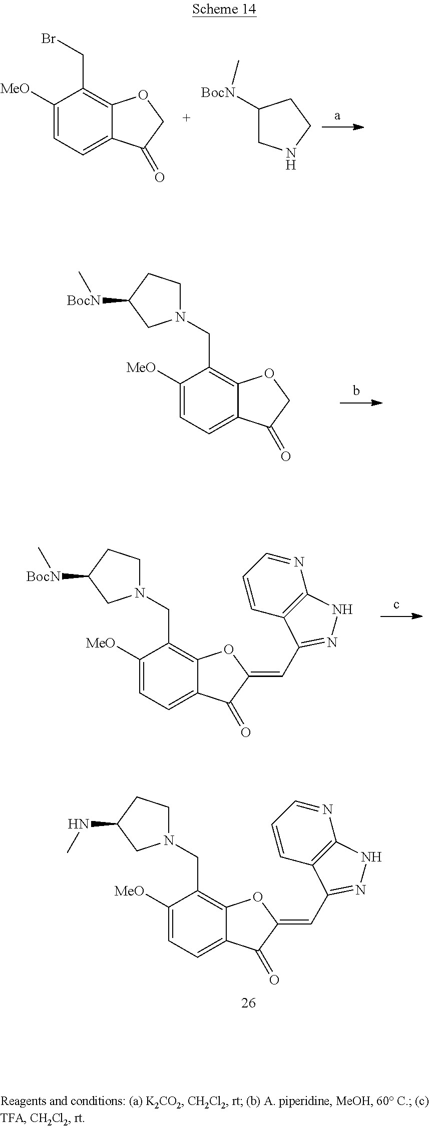 [SMILES: COc1ccc2c(c1CBr)OCC2=O ; CN(C(=O)OC(C)(C)C)C1CCNC1 ; COc1ccc2c(c1CN1CC[C@H](N(C)C(=O)OC(C)(C)C)C1)OCC2=O ; COc1ccc2c(c1CN1CC[C@H](N(C)C(=O)OC(C)(C)C)C1)O/C(=C\c1n[nH]c3ncccc13)C2=O ; CN[C@H]1CCN(Cc2c(OC)ccc3c2O/C(=C\c2n[nH]c4ncccc24)C3=O)C1]

(a) Step 1

Potassium carbonate (0.276 g, 2.00 mmol) and tert-butyl (S)-methyl(pyrrolidin-3-yl)carbamate (0.401 g, 2.00 mmol) were added to 8 mL of a methylene chloride solution of the 7-(bromomethyl)-6-methoxybenzofuran-3(2H)-one (0.514 g, 2.00 mmol) described in [WO2011/136319], and stirring was continued for 16 hours at room temperature. The reaction solution was filtered, the residue obtained by concentrating the filtrate was purified by silica gel chromatography (methanol/chloroform), and tert-butyl (S)-{1-[(6-methoxy-3-oxo-2,3-dihydrobenzofuran-7-yl)methyl]pyrrolidin-3-yl}(methyl)carbamate (0.246 g, 32%) was obtained.

¹H NMR (300 MHz, CDCl₃) δ 1.43 (s, 9H), 1.67-1.76 (m, 1H), 2.03-2.14 (m, 1H), 2.51-2.56 (m, 2H), 2.69-2.76 (m, 2H), 2.78 (s, 3H), 3.72 (s, 2H), 3.93 (s, 3H), 4.63 (s, 2H), 4.73 (m, 1H), 6.70 (d, J=8.1 Hz, 1H), 7.61 (d, J=8.1 Hz, 1H).

(b) Step 2

The 1H-pyrazolo[3,4-b]pyridine-3-carbaldehyde (0.0391 g, 0.266 mmol) described in [WO2011/136319] and piperidine (0.0181 g, 0.213 mmol) were added to 1 mL of a methanol solution of tert-butyl (S)-{1-[(6-methoxy-3-oxo-2,3-dihydrobenzofuran-7-yl)methyl]pyrrolidin-3-yl}(methyl)carbamate (0.100 g, 0.266 mmol), and stirred for two hours at 60° C. The residue obtained by concentrating the reaction solution was purified by silica gel column chromatography (methanol/chloroform), and tert-butyl (S,Z)-[1-({2-[(1H-pyrazolo[3,4-b]pyridin-3-yl)methylene]-6-methoxy-3-oxo-2,3-dihydrobenzofuran-7-yl}methyl)pyrrolidin-3-yl](methyl)carbamate (0.0962 g, 71%) was obtained.

¹H NMR (300 MHz, DMSO-d₆) δ 1.31 (s, 9H), 1.59-1.65 (m, 1H), 1.94-2.06 (m, 1H), 2.54 (s, 3H), 2.59-2.82 (m, 4H), 3.81 (d, J=12.6, 1H), 3.87 (d, J=12.6, 1H), 3.98 (s, 3H), 4.57 (m, 1H), 7.02 (s, 1H), 7.08 (d, J=8.7 Hz, 1H), 7.39 (dd, J=4.5, 8.7 Hz, 1H), 7.80 (d, J=8.7 Hz, 1H), 8.64 (dd, J=1.5, 4.5 Hz, 1H), 9.06 (dd, J=1.5, 8.7 Hz, 1H), 14.40 (brs, 1H).

(c) Step 3

Four milliliters of trifluoroacetic acid was added to 8 mL of a methylene chloride solution of tert-butyl (S,Z)-[1-({2-[(1H-pyrazolo[3,4-b]pyridin-3-yl)methylene]-6-methoxy-3-oxo-2,3-dihydrobenzofuran-7-yl}methyl)pyrrolidin-3-yl](methyl)carbamate (0.0902 g, 0.178 mmol), and stirred for 16 hours at room temperature. The residue obtained by concentrating the reaction solution was made basic by adding 8 mL of saturated sodium hydrogen carbonate aqueous solution, and extracted five times by chloroform. The organic layer was dried with anhydrous sodium sulfate, and the solvent was distilled off. After washing with acetonitrile, the solid obtained was dried under reduced pressure, and the target (S,Z)-2-[(1H-pyrazolo[3,4-b]pyridin-3-yl)methylene]-6-methoxy-7-{[3-(methylamino)pyrrolidin-1-yl]methyl}benzofuran-3(2H)-one (0.0376 g, 52%) was obtained.

¹H NMR (300 MHz, DMSO-d₆) δ 1.39-1.49 (m, 1H), 1.85-1.97 (m, 1H), 2.16 (s, 3H), 2.31 (dd, J=5.7, 9.6 Hz, 1H), 2.60 (t, J=6.6 Hz, 2H), 2.81-2.87 (m, 1H), 3.06 (m, 1H), 3.82 (s, 2H), 3.98 (s, 3H), 7.03 (s, 1H), 7.07 (d, J=8.7 Hz, 1H), 7.37 (dd, J=4.5, 8.1 Hz, 1H), 7.79 (d, J=8.7 Hz, 1H), 8.63 (dd, J=1.5, 4.5 Hz, 1H), 9.05 (d, J=8.1 Hz, 1H).

### Synthesis Example 20

### Synthesis of compound 27 ((Z)-2-[(1H-pyrazolo[3,4-b]pyridin-3-yl)methylene]-6-methoxy-7-[1-(piperazin-1-yl)prop-2-yl]benzofuran-3(2H)-one

Compound 27 was synthesized by synthesis scheme 15 below.

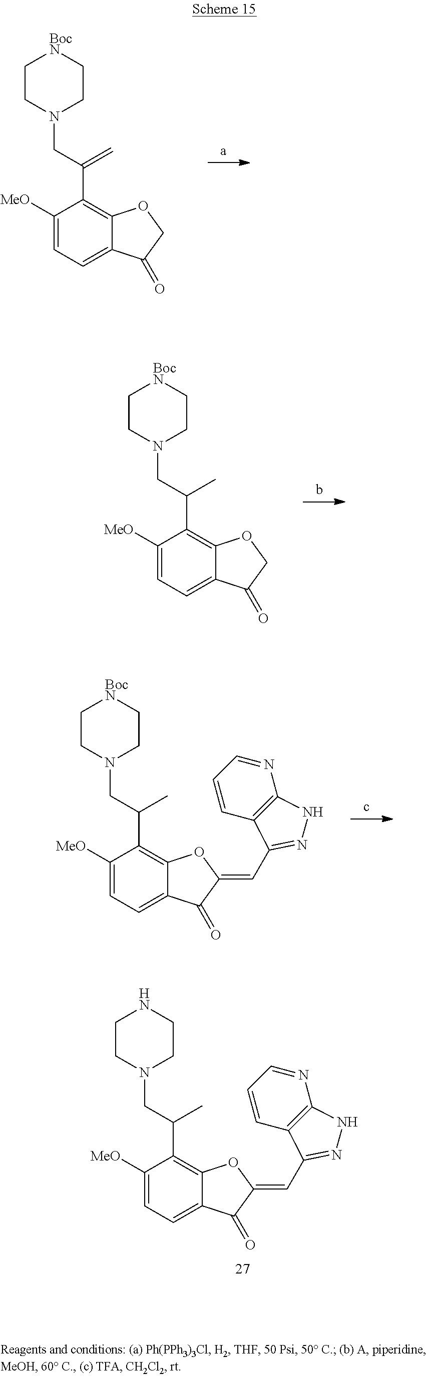 [SMILES: C=C(CN1CCN(C(=O)OC(C)(C)C)CC1)c1c(OC)ccc2c1OCC2=O ; COc1ccc2c(c1C(C)CN1CCN(C(=O)OC(C)(C)C)CC1)OCC2=O ; COc1ccc2c(c1C(C)CN1CCN(C(=O)OC(C)(C)C)CC1)O/C(=C\c1n[nH]c3ncccc13)C2=O ; COc1ccc2c(c1C(C)CN1CCNCC1)O/C(=C\c1n[nH]c3ncccc13)C2=O]

(a) Step 1

tert-Butyl 4-[2-(6-methoxy-3-oxo-2,3-dihydrobenzofuran-7-yl)allyl]piperazine-1-carboxylate (2.6 g, 6.7 mmol) synthesized by the same procedure as in steps 1, 2, and 3 of Synthesis Example 17 and chlorotris(triphenylphosphine)rhodium(I) (1.2 g, 1.3 mmol) were added to 200 mL of tetrahydrofuran, and stirred for 16 hours in a hydrogen atmosphere (50 Psi, 50° C.). After concentrating the reaction solution, it was diluted by 50 mL of ethyl acetate and 200 mL of methyl tert-butyl ether. The diluted solution was filtered by Celite, and the residue obtained by concentrating the filtrate was purified by silica gel chromatography (ethyl acetate/petroleum ether) to obtain tert-butyl 4-[2-(6-methoxy-3-oxo-2,3-dihydrobenzofuran-7-yl)propyl]piperazine-1-carboxylate (1.5 g, 58%).

¹H NMR (400 MHz, CDCl₃) δ 1.29 (d, J=6.8 Hz, 3H), 1.44 (s, 9H), 2.25-2.45 (m, 4H), 2.55-2.75 (m, 2H), 3.25-3.40 (m, 4H), 3.45-3.65 (m, 1H), 3.90 (s, 3H), 4.60 (s, 2H), 6.66 (d, J=8.8 Hz, 1H), 7.52 (d, J=8.8 Hz, 1H).

(b) Step 2

The 1H-pyrazolo[3,4-b]pyridine-3-carbaldehyde (0.0738 g, 0.500 mmol) described in [WO2011/136319] and piperidine (0.0341 g, 0.400 mmol) were added to 2 mL of a methanol solution of tert-butyl 4-[2-(6-methoxy-3-oxo-2,3-dihydrobenzofuran-7-yl)propyl]piperazine-1-carboxylate (0.195 g, 0.500 mmol), and stirred for two hours at 60° C. The reaction solution was cooled to room temperature, and the precipitated solid was filtered out to obtain tert-butyl (Z)-4-(2-{2-[(1H-pyrazolo[3,4-b]pyridin-3-yl)methylene]-6-methoxy-3-oxo-2,3-dihydrobenzofuran-7-yl}propyl)piperazine-1-carboxylate (0.191 g, 73%).

¹H NMR (300 MHz, DMSO-d₆) δ 1.34 (s, 9H), 1.38 (d, J=7.3 Hz, 3H), 2.27-2.40 (m, 4H), 2.68-2.81 (m, 2H), 3.16-3.20 (m, 4H), 3.62-3.69 (m, 1H), 3.96 (s, 3H), 7.02 (d, J=8.8 Hz, 1H), 7.13 (s, 1H), 7.33 (dd, J=4.4, 8.1 Hz, 1H), 7.71 (d, J=8.8 Hz, 1H), 8.63 (dd, J=1.5, 4.4 Hz, 1H), 8.77 (dd, J=1.5, 8.1 Hz, 1H), 14.31 (brs, 1H).

(c) Step 3

Six milliliters of trifluoroacetic acid was added to 12 mL of a methylene chloride solution of tert-butyl (Z)-4-(2-{2-[(1H-pyrazolo[3,4-b]pyridin-3-yl)methylene]-6-methoxy-3-oxo-2,3-dihydrobenzofuran-7-yl}propyl)piperazine-1-carboxylate (0.188 g, 0.362 mmol), and stirred for 18 hours at room temperature. The residue obtained by concentrating the reaction solution was made basic by adding 15 mL of saturated sodium hydrogen carbonate, and the precipitated solid was filtered out. The solid filtered out was washed by water and dried under reduced pressure, and the target (Z)-2-[(1H-pyrazolo[3,4-b]pyridin-3-yl)methylene]-6-methoxy-7-[1-(piperazin-1-yl)prop-2-yl]benzofuran-3(2H)-one (0.135 g, 89%) was obtained.

¹H NMR (300 MHz, DMSO-d₆) δ 1.38 (d, J=7.2 Hz, 3H), 2.30 (m, 4H), 2.58-2.73 (m, 4H), 3.21 (m, 2H), 3.63-3.70 (m, 1H), 3.95 (s, 3H), 7.02 (d, J=8.7 Hz, 1H), 7.12 (s, 1H), 7.27 (dd, J=4.5, 8.1 Hz, 1H), 7.70 (d, J=8.7 Hz, 1H), 8.59 (d, J=4.5 Hz, 1H), 8.78 (d, J=8.1 Hz, 1H).

### Synthesis Example 21

### Synthesis of compound 28 ((Z)-2-[(1H-pyrazolo[3,4-b]pyridin-3-yl)methylene]-6-chloro-7-(piperidin-4-ylmethyl)benzofuran-3(2H)-one)

Compound 28 was synthesized by synthesis scheme 16 below.

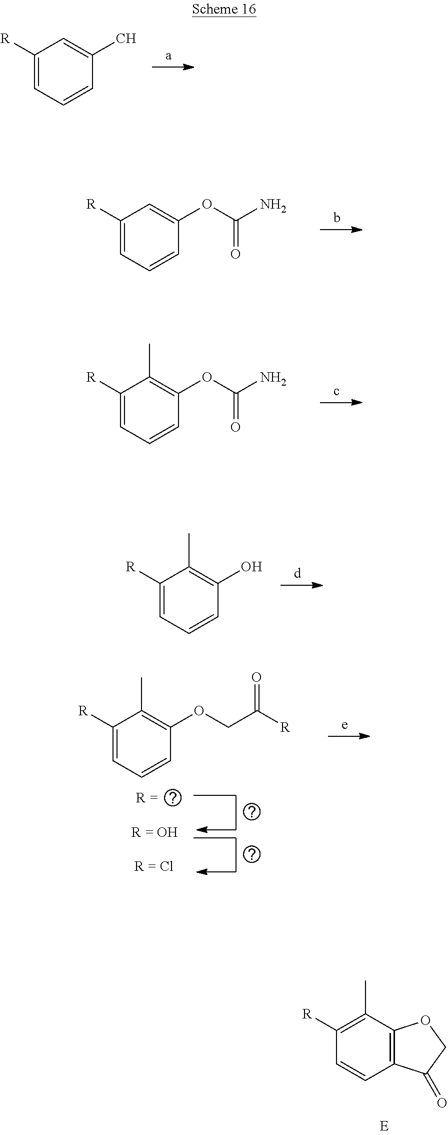

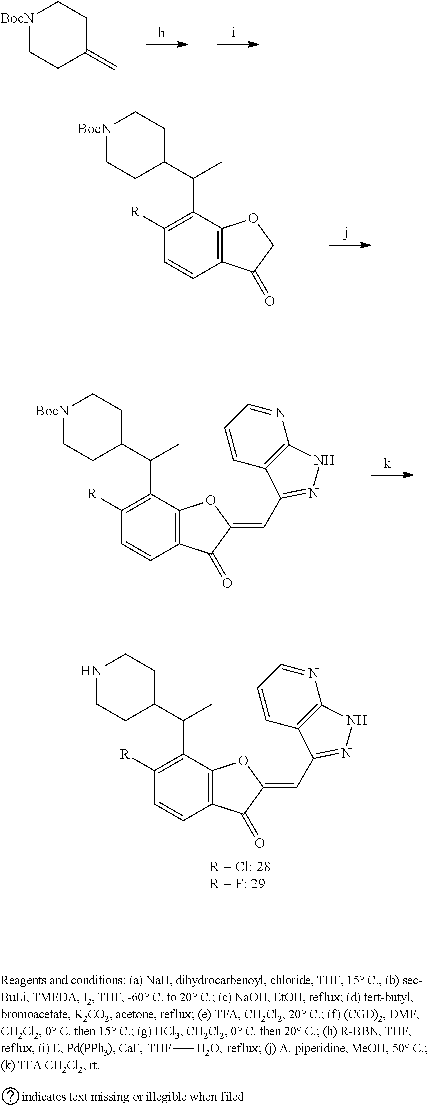

(a) Step 1

60% Sodium hydride (22.5 g, 562 mmol) was added over 30 minutes to 600 mL of a tetrahydrofuran solution of 3-chlorophenol (60.0 g, 468 mmol). The reaction solution was stirred for one hour at 15° C.; diethyl carbamic chloride (76.1 g, 561 mmol) was added, and the reaction solution was stirred for 3 hours at 15° C. The reaction solution was poured into 800 mL of ice water, and extracted twice by ethyl acetate. The combined organic layers were washed with water, and dried with anhydrous sodium sulfate. The residue obtained by distilling off the solvent was purified by silica gel chromatography (ethyl acetate/petroleum ether), and 3-chlorophenyl N,N-diethylcarbamate (98.0 g, 92%) was obtained.

¹H NMR (400 MHz, CDCl₃) δ 1.21-1.29 (m, 6H), 3.39-3.45 (m, 4H), 7.04-7.07 (m, 1H), 7.18-7.21 (m, 2H), 7.28-7.32 (m, 1H).

(b) Step 2

sec-Butyllithium (260 mL, 338 mmol, 1.3 M hexane solution) was added dropwise over one hour at −60° C. to 600 mL of a tetrahydrofuran solution of 3-chlorophenyl N,N-diethylcarbamate (59.0 g, 0.259 mmol) and N,N,N′,N′-tetramethylethylenediamine (TMEDA) (36.2 g, 312 mmol). After stirring for two hours at −60° C., 150 mL of a tetrahydrofuran solution of iodine (85.6 g, 338 mmol) was added dropwise over 30 minutes at −60° C. After dropwise addition had been completed, the reaction solution was heated to 20° C. over one hour, and the reaction was stopped by adding 800 mL of saturated sodium hydrogen thiosulfate aqueous solution. The reaction system was extracted twice by ethyl acetate, and the organic layer was washed with 2N hydrochloric acid, then with (saturated) brine. After drying by anhydrous sodium sulfate, the residue obtained by distilling off the solvent was purified by silica gel chromatography (ethyl acetate/petroleum ether), and 3-chloro-2-iodophenyl N,N-diethylcarbamate (57.0 g, 62%) was obtained.

¹H NMR (400 MHz, CDCl₃) δ 1.25 (t, J=7.2 Hz, 3H), 1.35 (t, J=7.2 Hz, 3H), 3.43 (q, J=7.2 Hz, 2H), 3.56 (q, J=7.2 Hz, 2H), 7.08 (dd, J=2.0, 7.2 Hz, 1H), 7.28-7.34 (m, 2H).

(c) Step 3

Sodium hydroxide (32.0 g, 800 mmol) was added at 25° C. to 400 mL of an ethanol solution of 3-chloro-2-iodophenyl N,N-diethylcarbamate (57.0 g, 161 mmol), and the reaction solution was heated and refluxed for two hours. The ethanol was distilled off under reduced pressure, and the residue was dissolved in 400 mL of water and extracted by petroleum ether. The water layer was neutralized by 2N hydrochloric acid, and extracted by ethyl acetate. The combined organic layers were washed with (saturated) brine, dried with anhydrous sodium sulfate, and the solvent was distilled off under reduced pressure to obtain 3-chloro-2-iodophenol (41.0 g, quantitative).

¹H NMR (400 MHz, CDCl₃) δ 5.58 (brs, 1H), 6.90 (dd, J=1.2, 8.4 Hz, 1H), 7.06 (dd, J=1.2, 8.0 Hz, 1H), 7.21 (t, J=8.0 Hz, 1H).

(d) Step 4

Potassium carbonate (26.7 g, 194 mmol) and tert-butyl bromoacetate (34.6 g, 177 mmol) were added to 400 mL of an acetone solution of 3-chloro-2-iodophenol (41.0 g, 161 mmol), and heated and refluxed for five hours. The reaction solution was filtered, and the crude product obtained by concentrating the filtrate was purified by silica gel chromatography (ethyl acetate/petroleum ether), and tert-butyl 2-(3-chloro-2-iodophenoxy)acetate (58.0 g, 97%) was obtained.

¹H NMR (400 MHz, CDCl₃) δ 1.50 (s, 9H), 4.61 (s, 2H), 6.58 (dd, J=1.2, 8.0 Hz, 1H), 7.15 (dd, J=1.2, 8.0 Hz, 1H), 7.23 (t, J=8.0 Hz, 1H).

(e) Step 5

Fifty milliliters of trifluoroacetic acid was added to 100 mL of a methylene chloride solution of tert-butyl 2-(3-chloro-2-iodophenoxy)acetate (55.0 g, 149 mmol), and stirred for four hours at 20° C. 2-(3-Chloro-2-iodophenoxy)acetic acid (45.7 g, 98%) was obtained by concentrating the reaction solution under reduced pressure.

¹H NMR (400 MHz, CDCl₃) δ 4.78 (s, 2H), 6.64 (dd, J=1.2, 8.0 Hz, 1H), 7.19 (dd, J=1.2, 8.0 Hz, 1H), 7.28 (t, J=8.0 Hz, 1H), 8.83 (brs, 1H).

(f) Step 6

Oxalyl chloride (33.4 g, 263 mmol) was added at 0° C. to 400 mL of a methylene chloride solution of 2-(3-chloro-2-iodophenoxy)acetic acid (41.0 g, 131 mmol). N,N-dimethylformamide (5 drops) was added next, and stirred for two hours at 15° C. The acid chloride (43.5 g, quantitative) obtained by concentrating the reaction solution under reduced pressure was used without purification in the next reaction.

(g) Step 7

Aluminum chloride (40.0 g, 300 mmol) was added over 20 minutes at 0° C. to 800 mL of a methylene chloride solution of the acid chloride (50.9 g, 154 mmol) obtained in step 6, and the reaction solution was stirred for 1.5 hour at 20° C. The reaction solution was poured into ice water, washed twice with 2N hydrochloric acid, and extracted twice by ethyl acetate. The combined organic layers were washed with (saturated) brine, dried with anhydrous sodium sulfate, and then concentrated under reduced pressure. The residue was subjected to silica gel chromatography (ethyl acetate/petroleum ether), and 6-chloro-7-iodobenzofuran-3(2H)-one (7.8 g, 17%) was obtained by suspending and washing the crude product obtained by methyl tert-butyl ether.

¹H NMR (400 MHz, CDCl₃) δ 4.81 (s, 2H), 7.59 (d, J=8.4 Hz, 1H), 7.26 (d, J=8.4 Hz, 1H).

(h) Step 8

9-BBN (30 mL, 15 mmol, 0.5 M tetrahydrofuran solution) was added dropwise to 80 mL of a tetrahydrofuran solution of the tert-butyl 4-methylenepiperidine-1-carboxylate (3.0 g, 15 mmol) described in [WO2011/136319]. After heating and refluxing for three hours, the solution was cooled to room temperature.

(i) Step 9

The 6-chloro-7-iodobenzofuran-3(2H)-one (2.94 g, 10 mmol) synthesized in step 7, cesium fluoride (4.56 g, 30 mmol), and water (32 mL) were added to the tetrahydrofuran solution prepared in step 8. Next, bis(tri-tert-butylphosphine)palladium(0) (0.260 mg, 0.5 mmol) was added, and refluxed for 12 hours. The reaction system was extracted twice by ethyl acetate, and the combined organic layers were washed with (saturated) brine, and dried with anhydrous sodium sulfate. The residue obtained by distilling off the solvent was subjected to silica gel chromatography (ethyl acetate/petroleum ether), and 1.8 g of the crude product obtained was purified by reverse-phase chromatography (water/acetonitrile) to obtain tert-butyl 4-[(6-chloro-3-oxo-2,3-dihydrobenzofuran-7-yl)methyl]piperidine-1-carboxylate (1.0 g, 27%).

¹H NMR (400 MHz, CDCl₃) δ 1.27-1.37 (m, 2H), 1.48 (s, 9H), 1.62 (d, J=12.0 Hz, 2H), 1.83-1.92 (m, 1H), 2.62-2.71 (m, 2H), 2.79 (d, J=7.2 Hz, 2H), 4.11 (brs, 2H), 4.70 (s, 2H), 7.13 (d, J=8.4 Hz, 1H), 7.48 (d, J=8.4 Hz, 1H).

(j) Step 10

The 1H-pyrazolo[3,4-b]pyridine-3-carbaldehyde (0.0736 g, 0.500 mmol) described in [WO2011/136319] and piperidine (0.0341 g, 0.400 mmol) were added to 2 mL of a methanol solution of tert-butyl 4-[(6-chloro-3-oxo-2,3-dihydrobenzofuran-7-yl)methyl]piperidine-1-carboxylate (0.183 g, 0.500 mmol), and stirred for two hours at 60° C. The reaction solution was cooled to room temperature, and the precipitated solid was filtered out to obtain tert-butyl (Z)-4-({2-[(1H-pyrazolo[3,4-b]pyridin-3-yl)methylene]-6-chloro-3-oxo-2,3-dihydrobenzofuran-7-yl}methyl)piperidine-1-carboxylate (0.191 g, 77%).

¹H NMR (300 MHz, DMSO-d₆) δ 1.11-1.23 (m, 2H), 1.32 (s, 9H), 1.62-1.66 (m, 2H), 1.90-2.04 (m, 1H), 2.59-2.69 (m, 2H), 2.96 (d, J=7.3 Hz, 2H), 3.88-3.92 (m, 2H), 7.18 (s, 1H), 7.38 (dd, J=5.1, 8.0 Hz, 1H), 7.44 (d, J=8.0 Hz, 1H), 7.73 (d, J=8.0 Hz, 1H), 8.65 (dd, J=1.5, 5.1 Hz, 1H), 8.88 (dd, 1.5, 8.0 Hz, 1H), 14.49 (brs, 1H).

(k) Step 11

Four milliliters of trifluoroacetic acid was added to 8 mL of a methylene chloride solution of tert-butyl (Z)-4-({2-[(1H-pyrazolo[3,4-b]pyridin-3-yl)methylene]-6-chloro-3-oxo-2,3-dihydrobenzofuran-7-yl}methyl)piperidine-1-carboxylate (0.188 g, 0.380 mmol), and stirred for 20 hours at room temperature. The residue obtained by concentrating the reaction solution was made basic by adding 10 mL of saturated sodium hydrogen carbonate aqueous solution, and the precipitated solid was filtered out. The solid filtered out was washed with water, and dried under reduced pressure, and the target (Z)-2-[(1H-pyrazolo[3,4-b]pyridin-3-yl)methylene]-6-chloro-7-(piperidin-4-ylmethyl)benzofuran-3(2H)-one (0.121 g, 81%) was obtained.

¹H NMR (300 MHz, DMSO-d₆) δ 1.42-1.55 (m, 2H), 1.80 (d, J=13.2 Hz, 2H), 2.05-2.12 (m, 1H), 2.74-2.83 (m, 2H), 2.99 (d, J=7.3 Hz, 2H), 3.24 (d, J=12.5 Hz, 2H), 7.21 (s, 1H), 7.45 (dd, J=4.4, 8.1 Hz, 1H), 7.47 (d, J=8.1 Hz, 1H), 7.76 (d, J=8.1 Hz, 1H), 8.68 (dd, J=1.5, 4.4 Hz, 1H), 8.88 (dd, 1.5, 8.1 Hz, 1H).

### Synthesis Example 22

### Synthesis of compound 29 ((Z)-2-[(1H-pyrazolo[3,4-b]pyridin-3-yl)methylene]-6-fluoro-7-(piperidin-4-ylmethyl)benzofuran-3(2H)-one)

Compound 29 was synthesized by synthesis scheme 16 above.

(a) Step 1

60% sodium hydride (55.6 g, 1.39 mol) was added over 30 minutes at 0° C. to 600 mL of a tetrahydrofuran solution of 3-fluorophenol (130 g, 1.16 mol). The reaction solution was stirred for one hour at 15° C. Diethyl carbamic chloride (188 g, 1.39 mol) was added, and the reaction solution was stirred for three hours at 15° C. The reaction solution was poured into 2000 mL of ice water, and extracted three times with ethyl acetate. The combined organic layers were washed with (saturated) brine, and dried with anhydrous sodium sulfate. The residue obtained by distilling off the solvent was purified by silica gel chromatography (ethyl acetate/petroleum ether), and 3-fluorophenyl N,N-diethylcarbamate (144 g, 59%) was obtained.

¹H NMR (400 MHz, CDCl₃) δ 1.18-1.33 (m, 6H), 3.36-3.54 (m, 4H), 6.88-6.98 (m, 3H), 7.29-7.36 (m, 1H).

(b) Step 2

sec-Butyllithium (473 mL, 615 mmol, 1.3 M hexane solution) was added dropwise over one hour at −60° C. to 1500 mL of a tetrahydrofuran solution of 3-fluorophenyl N,N-diethylcarbamate (100 g, 474 mmol) and N,N,N′,N′-tetramethylethylenediamine (66 g, 569 mmol). After stirring for two hours at −60° C., 200 mL of a tetrahydrofuran solution of iodine (156 g, 616 mmol) was added dropwise over 30 minutes at −60° C. After dropwise addition had been completed, the reaction solution was heated to 20° C. over one hour. The reaction was ended by adding 800 mL of saturated sodium hydrogen thiosulfate aqueous solution, and the reaction system was extracted three times with ethyl acetate. The organic layer was washed by 2N hydrochloric acid, then by (saturated) brine. After drying by anhydrous sodium sulfate, the residue obtained by distilling off the solvent was purified by silica gel chromatography (ethyl acetate/petroleum ether), and 3-fluoro-2-iodophenyl N,N-diethylcarbamate (110 g, 69%) was obtained.

¹H NMR (400 MHz, CDCl₃) δ 1.26 (t, J=7.2 Hz, 3H), 1.36 (t, J=7.2 Hz, 3H), 3.43 (q, J=7.2 Hz, 2H), 3.56 (q, J=7.2 Hz, 2H), 6.92-6.98 (m, 1H), 7.03 (d, J=8.0 Hz, 1H), 7.34 (m, 1H).

(c) Step 3

Sodium hydroxide (90.0 g, 2250 mmol) was added at 25° C. to 1500 mL of an ethanol solution of 3-fluoro-2-iodophenyl N,N-diethylcarbamate (153 g, 454 mmol), and the reaction solution was heated and refluxed for two hours. The ethanol was distilled off under reduced pressure, and the residue was dissolved in 1000 mL of water and extracted by petroleum ether. The water layer was neutralized by 2N hydrochloric acid, and extracted three times with ethyl acetate. The combined organic layers were washed with (saturated) brine, dried with anhydrous sodium sulfate, and the solvent was distilled off under reduced pressure to obtain a crude product of 3-fluoro-2-iodophenol (107 g).

(d) Step 4

The crude product of 3-fluoro-2-iodophenol (107 g) obtained in step 3 was dissolved in 1000 mL of acetone. Potassium carbonate (74.4 g, 539 mmol) and tert-butyl bromoacetate (95.9 g, 495 mmol) were added, and heated and refluxed for five hours. The reaction solution was filtered, and the crude product obtained by concentrating the filtrate was purified by silica gel chromatography (ethyl acetate/petroleum ether), and tert-butyl 2-(3-fluoro-2-iodophenoxy)acetate (156 g, 98%) was obtained.

¹H NMR (400 MHz, CDCl₃) δ 1.47-1.52 (m, 9H), 4.63 (s, 2H), 6.50 (d, J=8.3 Hz, 1H), 6.74-6.79 (m, 1H), 7.22-7.28 (m, 1H).

(e) Step 5

One hundred twenty milliliters of trifluoroacetic acid was added to 300 mL of a methylene chloride solution of tert-butyl 2-(3-fluoro-2-iodophenoxy)acetate (156 g, 443 mmol), and stirred for four hours at 20° C. The reaction solution was concentrated under reduced pressure to obtain 2-(3-fluoro-2-iodophenoxy) acetic acid (112 g, 85%).

¹H NMR (400 MHz, CDCl₃) δ 4.79 (s, 2H), 6.56 (d, J=8.3 Hz, 1H), 6.79-6.83 (m, 1H), 7.23-7.31 (m, 1H), 9.56 (brs, 1H).

(f) Step 6

Oxalyl chloride (96.9 g, 757 mmol) was added at 0° C. to 1000 mL of a methylene chloride solution of 2-(3-fluoro-2-iodophenoxy) acetic acid (112 g, 378 mmol). N,N-dimethylformamide (5 drops) was then added, and stirred for two hours at 15° C. The acid chloride (119 g, quantitative) obtained by concentrating the reaction solution under reduced pressure was used without further purification in the next reaction.

(g) Step 7

Aluminum chloride (101 g, 757 mmol) was added over 20 minutes at 0° C. to 1500 mL of a methylene chloride solution of the acid chloride (119 g, 378 mmol) obtained in step 6, and the reaction solution was stirred for 1.5 hour at 20° C. The reaction solution was poured into ice water, washed twice by 2N hydrochloric acid, and extracted twice by ethyl acetate. The combined organic layers were washed with (saturated) brine, dried with anhydrous sodium sulfate, and concentrated under reduced pressure. The residue was purified by silica gel chromatography (ethyl acetate/petroleum ether), and 6-fluoro-7-iodobenzofuran-3(2H)-one (9 g, 9%) was obtained.

¹H NMR (400 MHz, CDCl₃) δ 4.81 (s, 2H), 6.88 (d, J=8.4 Hz, 1H), 7.65-7.75 (m, 1H).

(h) Step 8

9-BBN (30 mL, 15 mmol, 0.5 M tetrahydrofuran solution) was added dropwise to 80 mL of a tetrahydrofuran solution of the tert-butyl 4-methylenepiperidine-1-carboxylate (3.0 g, 15 mmol) described in [WO2011/136319]. After heating and refluxing for three hours, the reaction system was cooled to room temperature.

(i) Step 9

The 6-fluoro-7-iodobenzofuran-3(2H)-one (2.78 g, 10 mmol) synthesized in step 7, cesium fluoride (4.56 g, 30 mmol), and water (32 mL) were added to the tetrahydrofuran solution prepared in step 8. Next, bis(tri-tert-butylphosphine)palladium(0) (0.500 mg, 0.98 mmol) was added, and refluxed for 12 hours. The reaction system was extracted twice by ethyl acetate. The combined organic layers were washed with (saturated) brine, and dried with anhydrous sodium sulfate. The residue obtained by distilling off the solvent was purified by silica gel chromatography (ethyl acetate/petroleum ether), and tert-butyl 4-[(6-fluoro-3-oxo-2,3-dihydrobenzofuran-7-yl)methyl]piperidine-1-carboxylate (1.2 g, 34%) was obtained.

¹H NMR (400 MHz, CDCl₃) δ 1.19-1.32 (m, 2H), 1.47 (s, 9H), 1.65 (brs, 1H), 1.73-1.92 (m, 2H), 2.66 (m, 4H), 4.01-4.20 (m, 2H), 4.70 (s, 2H), 6.80-6.85 (m, 1H), 7.56 (dd, J=5.6, 8.4 Hz, 1H).

(j) Step 10

The 1H-pyrazolo[3,4-b]pyridine-3-carbaldehyde (0.0230 g, 0.156 mmol) described in [WO2011/136319] and piperidine (0.0106 g, 0.125 mmol) were added to 1 mL of a methanol solution of tert-butyl 4-[(6-fluoro-3-oxo-2,3-dihydrobenzofuran-7-yl)methyl]piperidine-1-carboxylate (0.0546 g, 0.156 mmol), and stirred for two hours at 60° C. The reaction solution was cooled to room temperature, the precipitated solid was filtered out, and tert-butyl (Z)-4-({2-[(1H-pyrazolo[3,4-b]pyridin-3-yl)methylene]-6-fluoro-3-oxo-2,3-dihydrobenzofuran-7-yl}methyl)piperidine-1-carboxylate (0.0425 g, 57%) was obtained.

¹H NMR (300 MHz, DMSO-d₆) δ 1.10-1.23 (m, 2H), 1.35 (s, 9H), 1.64-1.69 (m, 2H), 1.83-1.95 (m, 1H), 2.57-2.73 (m, 2H), 2.83 (d, J=7.3 Hz, 2H), 3.89-3.93 (m, 2H), 7.15 (s, 1H), 7.19-7.25 (m, 1H), 7.38 (dd, J=4.4, 8.0 Hz, 1H), 7.80 (dd, J=5.9, 8.8 Hz, 1H), 8.65 (dd, J=1.5, 4.4 Hz, 1H), 8.88 (dd, 1.5, 8.0 Hz, 1H), 14.49 (brs, 1H).

(k) Step 11

Two milliliters of trifluoroacetic acid was added to 4 mL of a methylene chloride solution of tert-butyl (Z)-4-({2-[(1H-pyrazolo[3,4-b]pyridin-3-yl)methylene]-6-fluoro-3-oxo-2,3-dihydrobenzofuran-7-yl}methyl)piperidine-1-carboxylate (0.0412 g, 0.0860 mmol), and stirred for 16 hours at room temperature. The residue obtained by concentrating the reaction solution was made basic by adding 8 mL of saturated sodium hydrogen carbonate aqueous solution, and the precipitated solid was filtered out. The solid filtered out was washed with water and dried under reduced pressure to obtain the target (Z)-2-[(1H-pyrazolo[3,4-b]pyridin-3-yl)methylene]-6-fluoro-7-(piperidin-4-ylmethyl)benzofuran-3(2H)-one (0.0215 g, 77%).

¹H NMR (300 MHz, DMSO-d₆) δ 1.32-1.45 (m, 2H), 1.76-1.80 (m, 2H), 1.91-2.04 (m, 1H), 2.68-2.77 (m, 2H), 2.85 (d, J=7.3 Hz, 2H), 3.19 (d, J=12.5 Hz, 2H), 7.18 (s, 1H), 7.21-7.27 (m, 1H), 7.43 (dd, J=4.4, 8.1 Hz, 1H), 7.82 (dd, J=5.1, 8.1 Hz, 1H), 8.67 (dd, J=1.5, 4.4 Hz, 1H), 8.88 (dd, 1.5, 8.1 Hz, 1H).

### Synthesis Example 23

### Synthesis of compound 31 ((R,Z)-2-[(1H-pyrazolo[3,4-b]pyridin-3-yl)methylene]-7-[(2-(aminomethyl)pyrrolidin-1-yl)methyl]-6-methoxybenzofuran-3(2H)-one)

Compound 31 was synthesized was synthesis scheme 17 below.

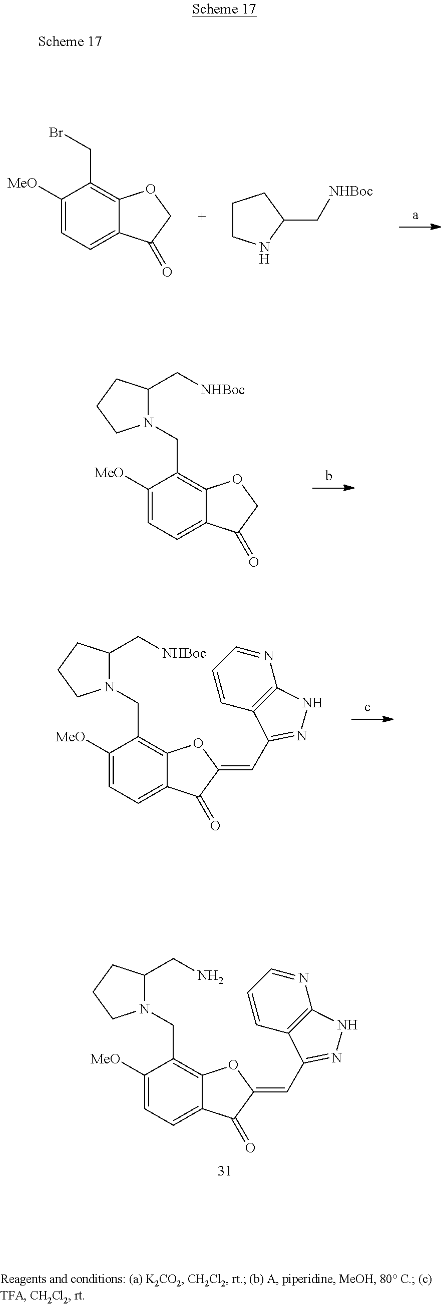 [SMILES: COc1ccc2c(c1CBr)OCC2=O ; CC(C)(C)OC(=O)NCC1CCCN1 ; COc1ccc2c(c1CN1CCCC1CNC(=O)OC(C)(C)C)OCC2=O ; COc1ccc2c(c1CN1CCCC1CNC(=O)OC(C)(C)C)O/C(=C\c1n[nH]c3ncccc13)C2=O ; COc1ccc2c(c1CN1CCCC1CN)O/C(=C\c1n[nH]c3ncccc13)C2=O]

(a) Step 1

Potassium carbonate (0.276 g, 2.00 mmol) and tert-butyl (R)-(pyrrolidin-2-ylmethyl)carbamate (0.401 g, 2.00 mmol) were added to 8 mL of a methylene chloride solution of the 7-(bromomethyl)-6-methoxybenzofuran-3(2H)-one (0.514 g, 2.00 mmol) described in [WO2011/136319], and stirring was continued for 16 hours at room temperature. The reaction solution was filtered, and the residue obtained by concentrating the filtrate was purified by silica gel chromatography (methanol/chloroform) to obtain tert-butyl (R)-({1-[(6-methoxy-3-oxo-2,3-dihydrobenzofuran-7-yl)methyl]pyrrolidin-2-yl}methyl)carbamate (0.212 g, 28%).

¹H NMR (300 MHz, DMSO-d₆) δ 1.39 (s, 9H), 1.47-1.56 (m, 3H), 1.71-1.80 (m, 1H), 2.24-2.33 (m, 1H), 2.54-2.59 (m, 1H), 2.73-2.78 (m, 1H), 2.84-2.92 (m, 1H), 3.17-3.25 (m, 1H), 3.47 (d, J=12.5 Hz, 1H), 3.82 (d, J=12.5 Hz, 1H), 3.92 (s, 3H), 4.77 (s, 2H), 6.32 (t, J=5.1 Hz, 1H), 6.89 (d, J=8.8 Hz, 1H), 7.58 (d, J=8.8 Hz, 1H).

(b) Step 2

The 1H-pyrazolo[3,4-b]pyridine-3-carbaldehyde (0.0365 g, 0.248 mmol) described in [WO2011/136319] and piperidine (0.0169 g, 0.198 mol) were added to 1 mL of a methanol solution of tert-butyl (R)-({1-[(6-methoxy-3-oxo-2,3-dihydrobenzofuran-7-yl)methyl]pyrrolidin-2-yl}methyl)carbamate (0.0935 g, 0.248 mmol), and stirred for two hours at 60° C. The residue obtained by concentrating the reaction solution was purified by silica gel column chromatography (methanol/chloroform), and tert-butyl (R,Z)-{[1-({2-[(1H-pyrazolo[3,4-b]pyridin-3-yl)methylene]-6-methoxy-3-oxo-2,3-dihydrobenzofuran-7-yl}methyl)pyrrolidin-2-yl]methyl}carbamate (0.0810 g, 64%) was obtained.

¹H NMR (300 MHz, DMSO-d₆) δ 1.32 (S, 9H), 1.47-1.56 (m, 3H), 1.72-1.83 (m, 1H), 2.34-2.42 (m, 1H), 2.64-2.70 (m, 1H), 2.78-2.89 (m, 2H), 3.12-3.19 (m, 1H), 3.73 (d, J=12.5 Hz, 1H), 4.00 (s, 3H), 4.04 (d, J=12.5 Hz, 1H), 6.28 (t, J=5.1 Hz, 1H), 7.06-7.08 (m, 2H), 7.39 (dd, J=4.4, 8.1 Hz, 1H), 7.81 (d, J=8.8 Hz, 1H), 8.63 (d, J=4.4 Hz, 1H), 8.98 (dd, J=1.5, 8.1 Hz, 1H).

(c) Step 3

One milliliter of trifluoroacetic acid was added to 2 mL of a methylene chloride solution of tert-butyl (R,Z)-{[1-({2-[(1H-pyrazolo[3,4-b]pyridin-3-yl)methylene]-6-methoxy-3-oxo-2,3-dihydrobenzofuran-7-yl}methyl)pyrrolidin-2-yl]methyl}carbamate (0.0777 g, 0.154 mmol), and stirred for 18 hours at room temperature. The residue obtained by concentrating the reaction solution was made basic by adding 6 mL of saturated sodium hydrogen carbonate aqueous solution, and the precipitated solid was filtered out. The solid obtained was washed with water and dried under reduced pressure to obtain the target (R,Z)-2-[(1H-pyrazolo[3,4-b]pyridin-3-yl)methylene]-7-{[2-(aminomethyl)pyrrolidin-1-yl]methyl}-6-methoxybenzofuran-3(2H)-one (0.0358 g, 57%).

¹H NMR (300 MHz, DMSO-d₆) δ 1.46-1.65 (m, 3H), 1.74-1.84 (m, 1H), 2.27-2.42 (m, 2H), 2.66-2.82 (m, 3H), 3.67 (d, J=12.6 Hz, 1H), 3.97 (s, 3H), 4.06 (d, J=12.6 Hz, 1H), 7.05 (d, J=8.8 Hz, 1H), 7.12 (s, 1H), 7.29 (dd, J=4.4, 8.1 Hz, 1H), 7.78 (d, J=8.8 Hz, 1H), 8.56 (dd, J=1.5, 4.4 Hz, 1H), 8.85 (d, J=8.1 Hz, 1H).

### Synthesis Example 24

### Synthesis of compound 32 ((S,Z)-2-[(1H-pyrazolo[3,4-b]pyridin-3-yl)methylene]-7-[(3-aminopiperidin-1-yl)methyl]-6-methoxybenzofuran-3(2H)-one

Compound 32 was synthesized by synthesis scheme 18 below.

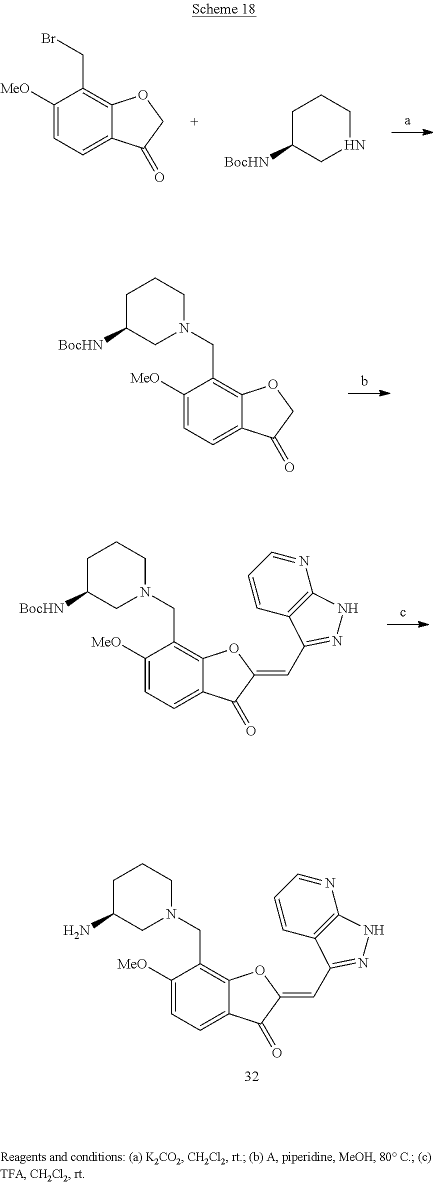 [SMILES: COc1ccc2c(c1CBr)OCC2=O ; CC(C)(C)OC(=O)N[C@H]1CCCNC1 ; COc1ccc2c(c1CN1CCC[C@H](NC(=O)OC(C)(C)C)C1)OCC2=O ; COc1ccc2c(c1CN1CCC[C@H](NC(=O)OC(C)(C)C)C1)O/C(=C\c1n[nH]c3ncccc13)C2=O ; COc1ccc2c(c1CN1CCC[C@H](N)C1)O/C(=C\c1n[nH]c3ncccc13)C2=O]

(a) Step 1

Triethylamine (0.223 g, 2.20 mmol) and tert-butyl (S)-piperidin-3-ylcarbamate (0.441 g, 2.20 mmol) were added to 8 mL of a methylene chloride solution of the 7-(bromomethyl)-6-methoxybenzofuran-3(2H)-one (0.514 g, 2.00 mmol) described in [WO2011/136319], and stirring was continued for 14 hours at room temperature. The residue obtained by concentrating the reaction solution was purified by silica gel chromatography (methanol/chloroform), and tert-butyl (S)-{1-[(6-methoxy-3-oxo-2,3-dihydrobenzofuran-7-yl)methyl]piperidin-3-yl}carbamate (0.499 g, 67%) was obtained.

¹H NMR (300 MHz, CDCl₃) δ 1.43 (s, 9H), 1.47-1.54 (m, 4H), 2.30-2.54 (m, 4H), 3.62 (s, 2H), 3.68-3.73 (m, 1H), 3.93 (s, 3H), 4.64 (s, 2H), 5.16 (m, 1H), 6.70 (d, J=8.8 Hz, 1H), 7.62 (d, J=8.8 Hz, 1H).

(b) Step 2

The 1H-pyrazolo[3,4-b]pyridine-3-carbaldehyde (0.0892 g, 0.606 mmol) described in [WO2011/136319] and piperidine (0.0413 g, 0.485 mmol) were added to 3 mL of a methanol solution of tert-butyl (S)-{1-[(6-methoxy-3-oxo-2,3-dihydrobenzofuran-7-yl)methyl]piperidin-3-yl}carbamate (0.228 g, 0.606 mmol), and stirred for two hours at 60° C. The reaction solution was cooled to room temperature, and the precipitated solid was filtered out to obtain tert-butyl (S,Z)-[1-({2-[(1H-pyrazolo[3,4-b]pyridin-3-yl)methylene]-6-methoxy-3-oxo-2,3-dihydrobenzofuran-7-yl}methyl)piperidin-3-yl]carbamate (0.232 g, 76%).

¹H NMR (300 MHz, DMSO-d₆) δ 1.09-1.17 (m, 2H), 1.31 (s, 9H), 1.58-1.69 (m, 2H), 1.88-2.06 (m, 2H), 2.73-2.80 (m, 1H), 2.86-2.91 (m, 1H), 3.35-3.43 (m, 1H), 3.72 (s, 2H), 3.97 (s, 3H), 6.63-6.66 (m, 1H), 7.03 (s, 1H), 7.08 (d, J=8.8 Hz, 1H), 7.41 (dd, J=4.4, 8.1 Hz, 1H), 7.81 (d, J=8.8 Hz, 1H), 8.60 (d, J=4.4 Hz, 1H), 9.01 (dd, J=1.5, 8.1 Hz, 1H), 14.39 (brs, 1H).

(c) Step 3

Four milliliters of trifluoroacetic acid was added to 8 mL of a methylene chloride solution of tert-butyl (S,Z)-[1-({2-[(1H-pyrazolo[3,4-b]pyridin-3-yl)methylene]-6-methoxy-3-oxo-2,3-dihydrobenzofuran-7-yl}methyl)piperidin-3-yl]carbamate (0.230 g, 0.455 mmol), and stirred for 24 hours at room temperature. The residue obtained by concentrating the reaction solution was made basic by adding saturated sodium hydrogen carbonate aqueous solution, and extracted three times by chloroform. The organic layer was dried with anhydrous sodium sulfate, and the solvent was distilled off under reduced pressure to obtain the target (S,Z)-2-[(1H-pyrazolo[3,4-b]pyridin-3-yl)methylene]-7-[(3-aminopiperidin-1-yl)methyl]-6-methoxybenzofuran-3(2H)-one (0.0759 g, 41%).

¹H NMR (300 MHz, DMSO-d₆) δ 0.94-1.06 (m, 1H), 1.35-1.46 (m, 1H), 1.60-1.73 (m, 2H), 1.80-1.87 (m, 1H), 2.02-2.09 (m, 1H), 2.62-2.69 (m, 1H), 2.76-2.80 (m, 1H), 2.85-2.89 (m, 1H), 3.70 (s, 2H), 3.97 (s, 3H), 7.03 (s, 1H), 7.08 (d, J=8.8 Hz, 1H), 7.38 (dd, J=4.4, 8.1 Hz, 1H), 7.80 (d, J=8.8 Hz, 1H), 8.63 (d, J=4.4 Hz, 1H), 9.02 (d, J=8.8 Hz, 1H).

### Example 1

### Measurement of PIM-3 Kinase Inhibitory Activity

1.5 μM of fluorescent substrate peptide (FAM-AKRRRLSSLRA-COOH), 30 μM of ATP, and 0.0833 ng/μL of PIM-3 kinase were incubated with a compound for 2 hours at room temperature, and the Pim-3 kinase activity was measured. The reaction was quantified by separating the substrate and phosphorylation reaction product by capillary electrophoresis (using a Perkin-Elmer EZ reader II) based on the difference in their charges. The results are shown below in Tables 1 and 2.

TABLE 1

| | Pim-3 IC₅₀ |
|---|---|
| Compound no. | (nM) |
| 1 | 29 |
| 2 | 2.7 |
| 3 | 71 |
| 4 | 0.60 |
| 5 | 1.8 |
| 6 | 7.6 |
| 7 | 2.8 |
| 8 | 38 |
| 9 | 8.4 |
| 10 | 19 |
| 11 | 1.4 |
| 12 | 7.9 |
| 13 | 150 |
| 14 | 1.0 |
| 15 | 1.6 |
| 16 | 0.70 |
| 17 | 2.2 |
| 18 | 0.35 |
| 19 | 0.70 |

TABLE 2

| | Pim-3 IC₅₀ |
|---|---|
| Compound no. | (nM) |
| 22 | 1.3 |
| 23 | 0.62 |
| 24 | 12 |
| 25 | 0.29 |
| 26 | 1.1 |
| 27 | 6.4 |
| 28 | 1.3 |
| 29 | 4.5 |
| 31 | 0.96 |
| 32 | 0.51 |

### Example 2

### Measurement of Growth Inhibitory Activity on Human Pancreatic Cancer Cell Lines

Two thousand cells of human pancreatic cancer cells lines (L3.6pl, MiaPaca-2, PANC-1, PCI35, PCI66) were seeded in a medium volume of 100 μL in each well of a 96-well microtiter plate. The medium was removed after 18 hours, and 100 μL/well of medium containing the test compound was added. Culture was continued for 72 or 96 hours. The medium containing the test compound was removed, and 100 μL/well of medium containing 10% WST-8 (Dojindo Laboratories Cell Counting kit-8) was added. After culturing for one hour, the OD 450 nm absorbance was measured, and the cell growth inhibitory activity was calculated. Furthermore, RPMI-1640 medium (Sigma) was used as the medium with 10% fetal calf serum (Invitrogen), 50 U/mL penicillin G (Sigma), and 50 μg/mL streptomycin (Sigma) added. The cell lines were cultured at 37° C. in a carbon dioxide incubator with a carbon dioxide concentration of 5%.

Tables 3 and 4 show the results of compound evaluation when cultured for 96 hours. Table 5 shows the results of compound evaluation when cultured for 72 hours.

TABLE 3

| | L3.6pl IC₅₀ | MiaPaca-2 IC₅₀ | PANC-1 IC₅₀ |
|---|---|---|---|
| Compound no. | (μM) | (μM) | (μM) |
| 2 | 2.8 | 8.6 | 7.4 |
| 4 | 1.7 | 0.93 | 3.1 |
| 5 | 3.4 | 2.5 | 5.4 |

TABLE 4

| | MiaPaca-2 IC₅₀ | PCI35 IC₅₀ | PCI66 IC₅₀ |
|---|---|---|---|
| Compound no. | (μM) | (μM) | (μM) |
| 6 | 1.6 | 1.8 | 1.8 |

TABLE 5

| | L3.6pl IC₅₀ | MiaPaca-2 IC₅₀ | PANC-1 IC₅₀ |
|---|---|---|---|
| Compound no. | (μM) | (μM) | (μM) |
| 8 | 3.5 | 3.5 | 12 |
| 9 | 4.0 | 3.0 | 12 |
| 10 | 7.5 | 3.0 | 14 |
| 11 | 3.5 | 3.4 | 8.3 |
| 12 | 3.5 | 3.2 | 8.2 |
| 13 | 3.5 | 2.4 | 8.4 |
| 18 | 0.25 | 0.22 | 0.42 |
| 19 | 0.30 | 0.22 | 0.33 |

### Example 3

### Animal Study

PCI66 cells, which are human pancreatic cancer cells, were suspended in Hanks balanced salt solution in a concentration of 2×10⁷ cells/mL, and 100 μL of the cell suspension was injected subcutaneously to the backs of BALB/c nu/nu mice (SLC, Shizuoka, Japan). Drug administration was begun 14 days after tumor injection. The drug was administered once a day for five days intraperitoneally, then withheld for two days. This cycle was repeated three times. The tumor volume was calculated by the following formula.

Tumor volume(cm³)=[maximum diameter](cm)×[minimum diameter]²(cm)/2

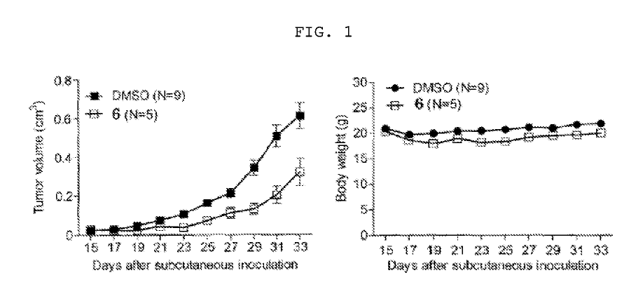

FIG. 1 shows the results obtained by intraperitoneal administration of 30 mg/kg of compound 6.

The left side of FIG. 1 shows the tumor volume after administration of the drug; the right side shows the changes in body weight after administration of the drug. Administration of compound 6 was found to suppress an increase in tumor volume.

### Example 4

### Measurement of Growth Inhibitory Activity on Human Pancreatic Cancer Cell Lines

Three thousand cells of human pancreatic cancer cell lines (L3.6pl, MiaPaca-2, PANC-1, PCI35, and PCI66) were seeded in a medium volume of 90 μL in each well of a 96-well microtiter plate. After 24 hours, 10 μL/well of medium containing the test compound was added. Culture was continued for 72 hours, and 10 μL/well of WST-8 (Dojindo Laboratories Cell Counting kit-8) was added. After culturing for 2 hours, the OD 450 nm absorbance was measured, and the cell growth inhibitory effect was calculated. The cell lines were cultured in a carbon dioxide incubator with a carbon dioxide concentration of 5%.

The results are shown in Table 6.

TABLE 6

| | L3.6pl | MiaPaca-2 | PANC-1 | PCI35 | PCI66 |
|---|---|---|---|---|---|
| Compound | IC₅₀ | IC₅₀ | IC₅₀ | IC₅₀ | IC₅₀ |
| no. | (μM) | (μM) | (μM) | (μM) | (μM) |
| 17 | 6.2 | 3.2 | 9.7 | 9.3 | 18 |
| 22 | 2.8 | 1.3 | 2.7 | 1.8 | 2.5 |
| 23 | 9.8 | 3.8 | 8.0 | 5.3 | 4.2 |
| 25 | 2.9 | 1.8 | 9.7 | 2.8 | 3.6 |
| 29 | 3.8 | 3.5 | 3.3 | 6.8 | 3.9 |
| 32 | 3.3 | 2.9 | 8.1 | 5.3 | 4.2 |

1. A PIM-3 kinase inhibitor containing a compound represented by the following formula (I) or a pharmacologically acceptable salt, hydrate, or solvate thereof: 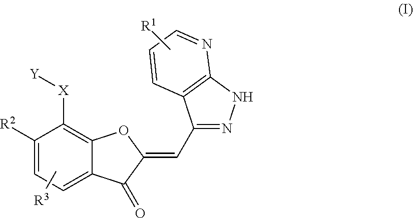
wherein
R¹ represents a hydrogen atom or from one to three identical or different substituents on the pyridine ring;
R² represents a hydrogen atom, a halogen, a hydroxyl group, a C₁₋₆ alkoxy group, a halo-substituted C₁₋₆ alkoxy group, an aryl-substituted C₁₋₆ alkoxy group, an aryloxy-substituted C₁₋₆ alkoxy group, a hydroxy-substituted C₁₋₆ alkoxy group, or a C₁₋₆ alkoxy-substituted C₁₋₆ alkoxy group;
R³ represents a hydrogen atom or one or two identical or different substituents on the benzene ring;
X is a methylene group or an ethylene group; the methylene group or the ethylene group may be substituted by from one to four C₁₋₄ alkyl groups or C₁₋₄ alkylene groups; and
Y represents a substituted or unsubstituted heterocyclic group.

2. The PIM-3 kinase inhibitor according to claim 1, wherein Y is a substituted or unsubstituted piperidine ring group, piperazine ring group, morpholine ring group, or pyrrolidine ring group.

3. The PIM-3 kinase inhibitor according to claim 1, wherein Y is a heterocyclic group substituted by at least one substituent selected from the group consisting of a halogen, a hydroxyl group, a C₁₋₆ alkyl group, an amino group, and an amino-substituted C₁₋₆ alkyl group, and wherein, if the heterocyclic group is substituted by two or more C₁₋₆ alkyl groups, then some of these alkyl groups may bond to each other to form a ring.

4. The PIM-3 kinase inhibitor according to claim 1, wherein R² is a hydrogen atom, a halogen, a hydroxyl group, a C₁₋₆ alkoxy group, a halo-substituted C₁₋₆ alkoxy group, a hydroxy-substituted C₁₋₆ alkoxy group, or a C₁₋₆ alkoxy-substituted C₁₋₆ alkoxy group.

5. A method for treating or preventing endoderm-derived organ cancers comprising administering a pharmaceutical composition containing a compound represented by the following formula (I) or a pharmacologically acceptable salt, hydrate, or solvate thereof: 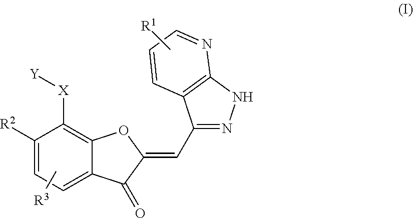
wherein
R¹ represents a hydrogen atom or from one to three identical or different substituents on the pyridine ring;
R² represents a hydrogen atom, a halogen, a hydroxyl group, a C₁₋₆ alkoxy group, a halo-substituted C₁₋₆ alkoxy group, an aryl-substituted C₁₋₆ alkoxy group, an aryloxy-substituted C₁₋₆ alkoxy group, a hydroxy-substituted C₁₋₆ alkoxy group, or a C₁₋₆ alkoxy-substituted C₁₋₆ alkoxy group;
R³ represents a hydrogen atom or one or two identical or different substituents on the benzene ring;
X is a methylene group or an ethylene group; the methylene group or the ethylene group may be substituted by from one to four C₁₋₄ alkyl groups or C₁₋₄ alkylene groups; and
Y represents a substituted or unsubstituted heterocyclic group.

6. The method according to claim 5, wherein the endoderm-derived organ cancer is pancreatic cancer.

7. A compound represented by the following formula (I) or a salt thereof: 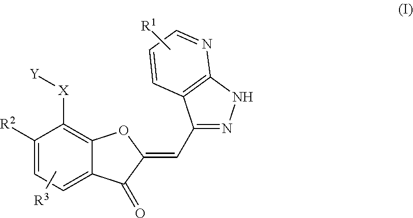
wherein
R¹ represents a hydrogen atom or from one to three identical or different substituents on the pyridine ring;
R² represents a hydrogen atom, a halogen, a hydroxyl group, a C₁₋₆ alkoxy group, a halo-substituted C₁₋₆ alkoxy group, an aryl-substituted C₁₋₆ alkoxy group, an aryloxy-substituted C₁₋₆ alkoxy group, a hydroxy-substituted C₁₋₆ alkoxy group, or a C₁₋₆ alkoxy-substituted C₁₋₆ alkoxy group;
R³ represents a hydrogen atom or one or two identical or different substituents on the benzene ring;
X is a methylene group or an ethylene group; the methylene group or the ethylene group may be substituted by from one to four C₁₋₄ alkyl groups or C₁₋₄ alkylene groups;
Y represents a substituted or unsubstituted heterocyclic group, with the proviso that the following compounds excluded

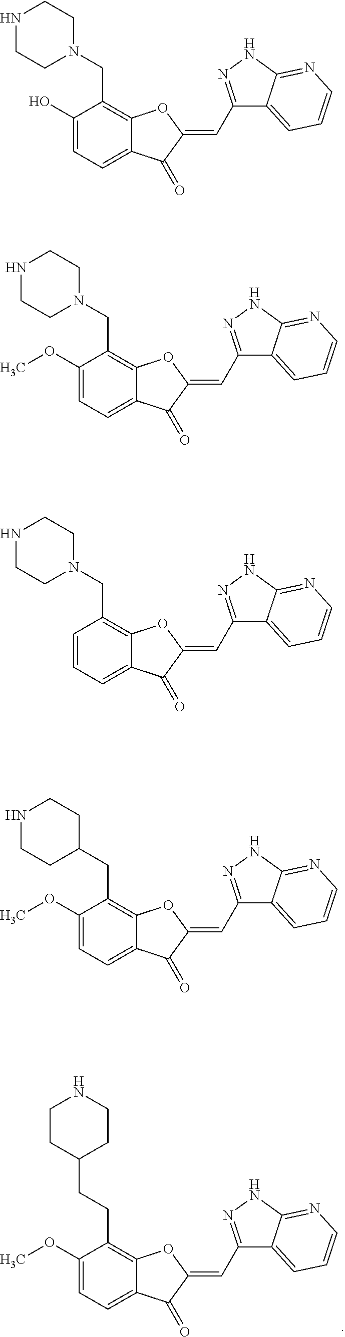 [SMILES: O=C1/C(=C/c2n[nH]c3ncccc23)Oc2c1ccc(O)c2CN1CCNCC1 ; COc1ccc2c(c1CN1CCNCC1)O/C(=C\c1n[nH]c3ncccc13)C2=O ; O=C1/C(=C/c2n[nH]c3ncccc23)Oc2c(CN3CCNCC3)cccc21 ; COc1ccc2c(c1CC1CCNCC1)O/C(=C\c1n[nH]c3ncccc13)C2=O ; COc1ccc2c(c1CCC1CCNCC1)O/C(=C\c1n[nH]c3ncccc13)C2=O]

8. The compound according to claim 7, wherein Y is a substituted or unsubstituted piperidine ring group, piperazine ring group, morpholine ring group, or pyrrolidine ring group.

9. The compound according to claim 7, wherein Y is a heterocyclic group substituted by at least one substituent selected from the group consisting of a halogen, a hydroxyl group, a C₁₋₆ alkyl group, an amino group, and an amino-substituted C₁₋₆ alkyl group, and wherein, if the heterocyclic group is substituted by two or more C₁₋₆ alkyl groups, then some of these alkyl groups may bond to each other to form a ring.

10. The compound according to claim 7, wherein Y is represented by the following formula (1), (2), or (3): 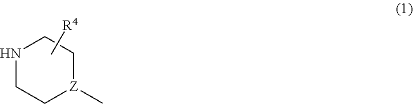
wherein R⁴ represents a hydrogen atom or from one to nine identical or different substituents on the six-membered ring; and Z represents a carbon or nitrogen; 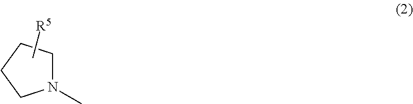
wherein R⁵ represents a hydrogen atom or from one to eight identical or different substituents on the pyrrolidine ring; 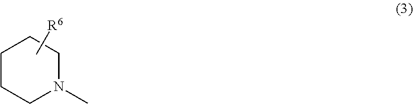
wherein R⁶ represents a hydrogen atom or from one to ten identical or different substituents on the piperidine ring.

11. The compound according to claim 10, wherein R⁴ is selected from the group consisting of a hydrogen atom, a halogen, a C₁₋₆ alkyl group, an amino group, and an amino-substituted C₁₋₆ alkyl group, and wherein, if R⁴ is two or more C₁₋₆ alkyl groups, then some of these alkyl groups may bond to each other to form a ring.

12. The compound according to claim 10, wherein R⁵ is selected from the group consisting of a hydrogen atom, a halogen, a C₁₋₆ alkyl group, an amino group, and an amino-substituted C₁₋₆ alkyl group, and wherein,
if R⁵ is two or more C₁₋₆ alkyl groups, then some of these alkyl groups may bond to each other to from a ring.

13. The compound according to claim 10, wherein R⁶ is selected from the group consisting of a hydrogen atom, a halogen, a C₁₋₆ alkyl group, an amino group, and an amino-substituted C₁₋₆ alkyl group, and wherein, if R⁶ is two or more C₁₋₆ alkyl groups, then some of these alkyl groups may bond to each other to form a ring.

14. The compound according to claim 7, wherein R² is selected from a hydrogen atom, a halogen, a hydroxyl group, a C₁₋₆ alkoxy group, a halo-substituted C₁₋₆ alkoxy group, a hydroxy-substituted C₁₋₆ alkoxy group, or a C₁₋₆ alkoxy-substituted C₁₋₆ alkoxy group.

15. The method according to claim 5, wherein the endoderm-derived organ cancer is pancreatic cancer.

16. The compound according to claim 12, wherein at least one R⁵ is an amino group or an amino-substituted C₁₋₆ alkyl group.
